(19) United States
(12) Patent Application Publication
(10) Pub. No.: US 2021/0231663 A1
(43) Pub. Date: Jul. 29, 2021

(54) MELANOMA CHECKPOINT INHIBITOR DETECTION AND TREATMENT

(71) Applicant: ONCIMMUNE GERMANY GMBH, Dortmund (DE)

(72) Inventors: Petra BUDDE, Dortmund (DE); Peter SCHULZ-KNAPPE, Hemmingen (DE); Hans-Dieter ZUCHT, Hannover (DE)

(73) Assignee: ONCIMMUNE GERMANY GMBH, Dortmund (DE)

(21) Appl. No.: 16/772,512

(22) PCT Filed: Dec. 10, 2018
(86) PCT No.: PCT/EP2018/084245

§ 371 Date: Jun. 12, 2020

Related U.S. Application Data
(60) Provisional application No. 62/747,181, filed on Oct. 18, 2018.
(60) Provisional application No. 62/597,720, filed on Dec. 12, 2017.

(51) Int. Cl. G01N 33/574; G01N 33/564; C07K 16/28

(52) U.S. Cl. CPC G01N 33/5743; C07K 16/2818; G01N 33/564

(57) ABSTRACT

Biomarkers are provided for predicting an irAE response associated with administration of anti-CTLA-4 antibodies (i.e. ipilumamb) and antibodies disrupting the PD-1/PD-L1 pathway (i.e. nivolumab or pembrolizumab) for treating melanoma. Methods of treatment incorporating such biomarkers are also provided.

### FIELD OF THE INVENTION

Biomarkers are provided for predicting an irAE response associated with administration of anti-CTLA-4 antibodies (i.e. ipilumamb) and antibodies disrupting the PD-1/PD-L1 pathway (i.e. nivolumab or pembrolizumab) for treating melanoma. Methods of treatment incorporating such biomarkers are also provided. Antibody responses towards tumor-associated antigens and self-antigens may have the potential to predict response, overall survival, and progression-free survival, receiving checkpoint inhibitor treatments.

### BACKGROUND OF THE INVENTION

Melanoma, also known as malignant melanoma, is a type of skin cancer that originates from the pigment-containing melanocytes. The main factors that predispose to the development of melanoma seem to be connected with overexposure to ultraviolet sunlight and a history of sunburn.

Melanoma is the least common but the most deadly skin cancer, accounting for only about 1% of all cases, but the dangerous form of skin cancer. According to the World Health Organization (WHO), about 132,000 melanoma skin cancers occur globally each year (http://www.who.int/uv/faq/skincancer/en/index1.html).

The survival rate for patients with melanoma depends on the thickness of the primary melanoma, whether the lymph nodes are involved, and whether the patient has developed metastasis at distant sites. The majority of patients initially present with stage I or II (localized melanoma), 8% have stage III (regional disease; and 4% have stage IV disease (distant metastases).

Surgery is the main treatment option for most melanomas, and usually cures early-stage melanomas. For many decades, patients with metastatic melanoma had a very poor prognosis with a median survival time of 8-9 month. Standard of care for unresectable stage III disease or stage IV melanoma was classical therapies such as chemotherapy and radiation.

Recent progress in tumor immunology research has led to a fourth therapy option that consists of approaches to stimulate the human immune system to identify and destroy developing tumor (cancer immunotherapy or immune-oncology treatment).

An effective immune response to cancer is dependent on the capacity to detect the tumor as foreign. Many tumor cells express abnormal proteins and molecules, which in theory should be recognized by the immune system. Proteins, which are present in the tumor and elicit an immune response, are called tumor-associated antigens (TAA). The group of TAA comprises mutated proteins, overexpressed or aberrantly expressed proteins, proteins produced by oncogenic viruses, germline-expressed proteins, glycoproteins or proteins, which are produced in small quantities or are not exposed to the immune system. The immune response to TAA includes cellular processes as well as the production of antibodies against TAA that lead to the elimination of tumor cells.

However, following prolonged antigen exposure the tumor can develop immune escape mechanisms that induce functionally exhausted T effector cells. Such immune escape mechanisms include down-regulation of MHC class I molecules on tumor cells to evade antigen-presentation to T effector cells. Another immune escape mechanism of tumor cells is the upregulation of PD-1 ligand (PD-L1, also called B7-H1) on tumor cells, which inhibits the function of tumor-infiltrating T cells. Such negative regulators of immune response pathways are collectively called immune checkpoints.

The development of therapeutic antibodies that modulate immune inhibitory pathway has been a major breakthrough in the treatment of melanoma. Currently, antibodies targeting the cytotoxic T-lymphocyte-associated antigen 4 (CTLA-4) and programmed death 1 (PD-1)/PD-L1 pathway have demonstrated improved survival in patients with advanced melanoma.

Immune checkpoints are negative regulators of T-cell immune function, when bound to their respective ligands CD80/86 and programmed cell-death ligand 1 and 2 (PD-L1/PD-L2).

In addition, drugs targeting other checkpoints such as lymphocyte activation gene 3 protein (LAG3), T cell immunoglobulin mucin 3 (TIM-3), and IDO (Indoleamine 2,3-dioxygenase) are in development.

Ipilimumab (Yervoy), an inhibitor of CTLA-4, is approved for the treatment of advanced or unresectable melanoma.

Nivolumab (Opdivo) and pembrolizumab (Keytruda), both PD-1 inhibitors, are approved to treat patients with advanced or metastatic melanoma.

Anti-PD-L1 inhibitor avelumab (Bavencio) has received orphan drug designation by the European Medicines Agency for the treatment of gastric cancer in January 2017. The US Food and Drug Administration (FDA) approved it in March 2017 for Merkel-cell carcinoma, an aggressive type of skin cancer.

Despite the fact that checkpoint inhibitors have greatly improved the survival of advanced metastatic melanoma, non-responsiveness is also observed with only about 30% of patients appear to benefit from ipilimumab (anti-CTLA-4) treatment (Callahan et al., 2013). Compared with ipilimumab antibodies targeting PD-1, nivolumumab and pembrolizumab, have shown increased efficacy in metastatic melanoma. Efficacy may be even further increased when using a combination of nivolumumab with ipilimumab, which is also approved for metastatic melanoma and has demonstrated a 2-year overall survival rate of 63.8% (Hodi et al., 2016).

The potent ability of checkpoint inhibitors to activate the immune system can result in tissue specific inflammation characterized as immune-related adverse events (irAEs). The main side effects include diarrhea, colitis, hepatitis, skin toxicities, arthritis, diabetes, endocrinopathies such as hypophysitis and thyroid dysfunction (Spain et al., 2016). In particular, the combination therapy of nivolumab with ipilimumab led to a rate of high-grade irAEs of 55%, compared with 27% or 16% for nivolumab or ipilimumab monotherapy, respectively (Larkin et al., 2015). Although infrequent, one of the most concerning effects of ipilimumab and combination therapies of ipilumumab, is the development of severe and even life-threatening colitis.

Therefore, biomarkers are needed to predict both clinical efficacy and toxicity. Such biomarkers may guide patient selection for both monotherapy and combination therapy (Topalian et al., 2016).

There are apparent differences between the CTLA-4 and PD-1 pathways of the immune response. CTLA-4 acts more globally on the immune response by stopping potentially autoreactive T cells at the initial stage of naive T-cell activation, typically in lymph nodes. The PD-1 pathway regulates previously activated T cells at the later stages of an immune response, primarily in peripheral tissues (Buchbinder and Desai, 2016).

Substantial efforts have been undertaking to identify biomarkers for predicting which patient will respond best to immune checkpoint inhibition.

Given the mechanism of action of inhibiting the PD-1 pathway, several studies have evaluated the expression of the PD-L1 ligand in the tumor as a biomarker of clinical response. However, differences regarding the predictive value of PD-L1 expression have been found. This limits the current use of PD-L1 as a biomarker for predicting clinical response. The differences in the utility of PDL1 as biomarker may be caused by differences in the assay type used in different studies and by variable expression of PD-L1 during therapy (Manson et al., 2016).

Since checkpoint inhibition is typically viewed as enhancing the activity of effector T cells in the tumor and tumor environment, other biomarker approaches have focused on identifying TAA recognized by T cells. However, this approach is limited to exploratory analyses and is not practical in a routine laboratory setting because it requires patient-specific MHC reagents (Gulley et al., 2014).

A largely overlooked immune cell type in the context of immunotherapies are B cells, which can exert both anti-tumor and tumor-promoting effects by providing co-stimulatory signals and inhibitory signals for T cell activation, cytokines, and antibodies (Chiaruttini et al., 2017).

Furthermore, B cells also express the immune checkpoint regulators PD-1, PD-L1, and CTLA-4 (Chiaruttini et al., 2017). Thus, administration of agents that modulate immune checkpoint molecules may also have effects on B cell activation and autoantibody production.

B-cells produce anti-tumor antibodies, which can mediate antibody-dependent cellular cytoxicity (ADCC) of tumor cells and activation of the complement cascade. It is well established that many cancer types induce an antibody response, which can be used for diagnostic purposes. Although some cancer patients shown an antibody response to neo-antigens restricted to the tumor, the majority of antibodies in cancer patients are directed to self-antigens and are therefore autoantibodies (Bei et al., 2009). Breakthrough of tolerance and elevated levels of autoantibodies to self-antigens are also a prominent feature of many autoimmune diseases.

Thus, autoantibodies hold the potential to serve as biomarkers of a sustained humoral anti-tumor response/non-response and irAE in cancer patients treated with immunotherapeutic approaches.

Compared to biomarker strategies involving the identification of TAA-specific T-cells, the identification of autoantibodies can be performed using modern multiplex high-throughput screening approaches using minimal amounts of serum (Budde et al., 2016).

### BRIEF DESCRIPTION OF DRAWINGS

FIG. 1 illustrates a design of the cancer screen.

KEGG Pathway Analysis ((Kyoto Encyclopedia of Genes and Genomes) of human (has) proteins and antigens included in the cancer autoantibody screen. Proteins were selected to represent the following three categories: natural and autoimmune antigens, tumor-associated antigens, immune-related pathways and dysregulated pathways in autoimmune diseases, cancer signaling pathways, and proteins or genes overexpressed in different cancer types. The individual categories are listed on the x-axis, with the number of proteins per category is indicated at the y-axis.

FIG. 2 illustrates the number of analyzed patients and serum samples per immune-oncology treatment, or therapy.

Pre-treatment samples were collected before initiation of therapy, and post-treatment samples were collected at approximately 3 and 6 month following treatment.

FIG. 3 illustrates the best response according to RECIST 1.1 for 193 melanoma patients in percentage per immune-oncology therapy.

PD: progressive disease, SD: stable disease, PR: partial response, and CR: complete response.

FIG. 4 illustrates IrAE for 193 melanoma patients in percentage per immune-oncology therapy.

The graph shows the percentage of all irAEs per treatment as well detailed information of specific irAEs.

FIG. 5 illustrates Box-and-Whisker plots and ROC curves of three autoantibodies in melanoma patients and healthy controls (HC).

Box-and-Whisker plots and ROC (Receiver operating characteristics) curves of IgG autoantibody reactivities against CREB3L, CXCL5, and NME1 in serum samples of melanoma patients and healthy controls. Numbers at the y-axis indicate the log 2 Luminex Median Fluorescence Intensity values (MFI).

FIG. 6 illustrates Box-and-Whisker plots of autoantibodies predicting DCR or PD to immune-oncology treatment in general.

Box-and-Whisker plots show a comparison of pre-treatment IgG autoantibody levels of patients with progressive disease (PD) and those achieving disease control rate (DCR). DCR is defined as CR, PR, or SD. Numbers at the y-axis indicate the log 2 Luminex Median Fluorescence Intensity values (MFI).

Pretreatment samples of patients treated with different checkpoint inhibitors (FIG. 2) are jointly analyzed.

FIG. 7 illustrates Box-and-Whisker plots and ROC curves of two baseline autoantibodies predicting irAE in melanoma patients.

Box-and-Whisker plots and ROC curves show a comparison of pre-treatment IgG autoantibody levels of patients who develop or do not develop irAEs following treatment with checkpoint inhibitors. Pretreatment samples of patients treated with different checkpoint inhibitors (FIG. 2) are jointly analyzed.

FIG. 8 illustrates Box-and-Whisker Plots of baseline autoantibodies predicting DCR or PD to ipilimumab.

Box-and-Whisker plots show a comparison of pre-treatment IgG autoantibody levels of patients with progressive disease (PD) and those achieving disease control rate (DCR). DCR is defined as CR, PR, or SD. Numbers at the y-axis indicate the log 2 Luminex Median Fluorescence Intensity values (MFI). Baseline (T0) samples of patients treated with anti-CTLA-4 blocker ipilimumab are analyzed.

FIG. 9 illustrates Box-and-Whisker plots baseline autoantibodies predicting irAE in ipilimumab-treated patients.

Box-and-Whisker plots show a comparison of pre-treatment IgG autoantibody levels of patients who develop or do not develop irAEs following treatment with checkpoint inhibitors. Pre-treatment (T0) samples) of patients treated with anti-CTLA-4 blocker ipilimumab are analyzed.

FIG. 10 illustrates Box-and-Whisker plots of baseline autoantibodies predicting DCR or PD to pembrolizumab.

Box-and-Whisker plots show a comparison of pre-treatment IgG autoantibody levels of patients with progressive disease (PD) and those achieving disease control rate (DCR). DCR is defined as CR, PR, or SD. Numbers at the y-axis indicate the log 2 Luminex Median Fluorescence Intensity values (MFI). Baseline (T0) samples of patients treated with anti-PD-1/PD-L1 pathway blocker pembrolizumab are analyzed.

FIG. 11 illustrates Box-and-Whisker Plots baseline autoantibodies predicting irAE in pembrolizumab-treated patients.

Box-and-Whisker plots show a comparison of pre-treatment IgG autoantibody levels of patients who develop or do not develop irAEs following treatment with checkpoint inhibitors. Pre-treatment (T0 samples) of patients treated with anti-CTLA-4 blocker pembrolizumab are analyzed.

FIG. 12 illustrates study samples and data analysis workflow.

For data mining patients were regrouped into the following modeling cohorts: “all treatments”=complete patient cohort; “ipi-ever”=patients treated with ipi-mono, ipi/nivo or pembro with prior ipi; “ipi-mono”=ipi-mono cohort; “pembro-never-ipi”=pembro-treated patients without prior ipi.

FIG. 13 illustrates summary statistics for 47 autoantibodies predicting irAE and colitis.

Autoantibodies predicting an adverse event (colitis are irAE) are highlighted in black, whereas those predicting a reduced risk are shown in white.

FIG. 14 illustrates Kaplan Meier curves with confidence intervals of baseline autoantibodies and their targets predicting colitis.

Serum autoantibody levels were dichotomized and Kaplan Meier curves for patients with high and low autoantibody levels plotted. X-axis: Time (days), and Y-axis: Event probability.

FIG. 15 illustrates Kaplan Meier curves with confidence intervals of baseline autoantibodies and their targets predicting irAE.

Serum autoantibody levels were dichotomized and Kaplan Meier curves for patients with high and low autoantibody levels plotted. X-axis: Time (days), and Y-axis: Event probability.

FIG. 16 illustrates optimized marker combinations for prediction of colitis (A) and irAE (B).

Filled circles: Positive predictive autoantibodies, grey circles: negative predictive autoantibodies

### SUMMARY OF THE INVENTION

Treatment with anti-CTLA-4 (i.e. ipilimumab), antibodies disrupting the PD-1/PD-L1 pathway (i.e. nivolumab or pembrolizumab) or combination therapies have demonstrated efficacy in melanoma. However, not all patients benefit equally, and administration of these antibodies can be associated with irAE. Thus, biomarkers to predict the response are urgently needed. Antibody responses towards tumor-associated antigens and self-antigens may have the potential to predict response, overall survival, and progression-free survival, receiving checkpoint inhibitor treatments.

In one aspect is provided a method of identifying a tumor-associated antigen (TAA) for melanoma. A group of patients with melanoma is selected. Also, a group of patients who are healthy are selected. A sample from at least one patient in the group with melanoma is assayed for the level of an autoantibody to an antigen. The level of the autoantibody to an antigen in the group of patients with melanoma is compared to the level of the autoantibody in the group of healthy patients. The antigen is determined to be a TAA for melanoma if the level of the autoantibody to the antigen is statistically different between the group of patients with melanoma versus the group of healthy patients.

Additional aspects and embodiments are described below in the Detailed Description.

### DETAILED DESCRIPTION OF THE INVENTION

Unless defined otherwise, technical and scientific terms used herein have the same meaning as commonly understood by one of ordinary skill in the art to which this invention belongs.

The terms “a,” “an,” and “the” do not denote a limitation of quantity, but rather denote the presence of “at least one” of the referenced item. In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

As used herein, “autoantibody” means an antibody produced by the immune system of a subject that is directed to, and specifically binds to an “autoantigen/self-antigen” or an “antigenic epitope” thereof. Specifically bind” and/or “specifically recognize” as used herein, refers to the higher affinity of a binding molecule for a target molecule compared to the binding molecule's affinity for non-target molecules. A binding molecule that specifically binds a target molecule does not substantially recognize or bind non-target molecules, e.g., an antibody “specifically binds” and/or “specifically recognize” another molecule, meaning that this interaction is dependent on the presence of the binding specificity of the molecule structure, e.g., an antigenic epitope.

As used herein, the term “epitope” refers to that portion of any molecule capable of being recognized by, and bound by, a T cell or an antibody (the corresponding antibody binding region may be referred to as a paratope), and/or eliciting an immune response. In general, epitopes consist of chemically active surface groupings of molecules, e.g., amino acids, and have specific three-dimensional structural characteristics as well as specific charge characteristics.

As used herein, the terms “diagnose” or “diagnosis” or “diagnosing” refers to determining the nature or the identity of a condition or disease or disorder, e.g., melanoma, detecting and/or classifying the melanoma in a subject. A diagnosis may be accompanied by a determination as to the severity of the melanoma. The term also encompasses assessing or evaluating the melanoma status (progression, regression, stabilization, response to treatment, etc.) in a patient known to have melanoma.

As used herein, the term “sample” refers to a sample obtained for evaluation in vitro. The sample can be any sample that is expected to contain antibodies and/or immune cells. The sample can be taken from blood, e.g., serum, peripheral blood, peripheral blood mononuclear cells (PBMC), whole blood or whole blood pre-treated with an anticoagulant such as heparin, ethylenediamine tetraacetic acid, plasma or serum. Sample can be pretreated prior to use, such as preparing plasma from blood, diluting viscous liquids, or the like; methods of treatment can also involve separation, filtration, distillation, concentration, inactivation of interfering components, and the addition of reagents.

Within the scope of this invention, the term “patient” is understood to mean any test subject (human or mammal), with the provision that the test subject is tested for melanoma.

As used herein, the terms “treat,” “treatment,” “treating,” or “amelioration” refer to therapeutic treatments, wherein the object is to reverse, alleviate, ameliorate, inhibit, slow down or stop the progression or severity of melanoma, an associated condition and/or a symptom thereof. The term “treating” includes reducing or alleviating at least one adverse effect or symptom of melanoma. Treatment is generally “effective” if one or more symptoms or clinical markers are reduced. Alternatively, or in addition, treatment is “effective” if the progression of a disease is reduced or halted.

In one aspect is provided a method of identifying a tumor-associated antigen (TAA) for melanoma cancer. A group of patients with melanoma is selected. Also, a group of patients who are healthy are selected. A sample from at least one patient in the group with melanoma is assayed for the level of an autoantibody to an antigen. The level of the autoantibody to an antigen in the group of patients with melanoma is compared to the level of the autoantibody in the group of healthy patients. The antigen is determined to be a TAA for melanoma if the level of the autoantibody to the antigen is statistically different between the group of patients with melanoma versus the group of healthy patients.

A patient or subject can be one who has been previously diagnosed with or identified as suffering from or under medical supervision for melanoma. A subject can be one who is diagnosed and currently being treated for, or seeking treatment, monitoring, adjustment or modification of an existing therapeutic treatment, or is at a risk of developing melanoma, e.g., due to family history, carrying alleles or genotype associated with melanoma, a history of excessive sun exposure, or development of moles and lesions associated with later development of melanoma.

Autoantibodies can be formed by a patient before melanoma progresses or otherwise shows symptoms. Early detection, diagnosis and also prognosis and (preventative) treatment would therefore be possible years before the visible onset of progression. Devices and means (arrangement, array, protein array, diagnostic tool, test kit) and methods described herein can enable a very early intervention compared with known methods, which considerably improves the prognosis and survival rates. Since the melanoma-associated autoantibody profiles change during the establishment and treatment/therapy of melanoma, the invention also enables the detection and the monitoring of melanoma at any stage of development and treatment and also monitoring within the scope of aftercare in the case of melanoma. The means according to the invention also allow easy handling at home by the patient himself and cost-effective routine precautionary measures for early detection and also aftercare.

Different patients may have different melanoma-associated autoantibody profiles, for example different cohorts or population groups differ from one another. Here, each patient may form one or more different melanoma-associated autoantibodies during the course of the development of melanoma and the progression of the disease of melanoma, that is to say also different autoantibody profiles. In addition, the composition and/or the quantity of the formed melanoma-associated autoantibodies may change during the course of the melanoma development and progression of the disease, such that a quantitative evaluation is necessary. The therapy/treatment of melanoma also leads to changes in the composition and/or the quantity of melanoma-associated autoantibodies. The large selection of melanoma-associated marker sequences according to the invention allows the individual compilation of melanoma-specific marker sequences in an arrangement for individual patients, groups of patients, certain cohorts, population groups, and the like. In an individual case, the use of a melanoma-specific marker sequence may therefore be sufficient, whereas in other cases at least two or more melanoma-specific marker sequences have to be used together or in combination in order to produce a meaningful autoantibody profile.

Compared with other biomarkers, the detection of melanoma-associated autoantibodies for example in the serum/plasma has the advantage of high stability and storage capability and good detectability. The presence of autoantibodies also is not subject to a circadian rhythm, and therefore the sampling is independent of the time of day, food intake and the like.

In addition, the melanoma-associated autoantibodies can be detected with the aid of the corresponding antigens/autoantigens in known assays, such as ELISA or Western Blot, and the results can be checked for this.

In some embodiments, the antigen is an antigen encoded by a gene listed in Table 1. In some embodiments, the antigen is an antigen encoded by a gene listed in Table 9. In some embodiments, the TAA is encoded by a gene listed in Table 2. In some embodiments, the antigen comprises an amino acid sequence of any one of SEQ ID NOS: 1-169.

In some embodiments, the assaying comprises contacting a portion of serum from the patient with a sample of an antigen immobilized onto a solid support. In some embodiments, the solid support is a bead. In some embodiments, the bead is a microsphere.

Various ways of performing the assay can be undertaken. A portion of serum from the patient with melanoma is contacted with a sample of an antigen. The antigen may be immobilized onto a solid support, in particular a filter, a membrane, a bead or small plate or bead, for example a magnetic or fluorophore-labelled bead, a silicon wafer, glass, metal, plastic, a chip, a mass spectrometry target or a matrix. A microsphere as a solid support may also be used. Multiple antigens may be coupled to multiple different solid supports and then arranged on an array.

The array may be in the form of a “protein array”, which in the sense of this invention is the systematic arrangement of melanoma-specific marker sequences on a solid support, wherein the melanoma-specific marker sequences are proteins or peptides or parts thereof, and wherein the support is preferably a solid support.

The sample comprising any of the TAAs, autoantigens, autoantibodies, are part of, found in, or otherwise present in, a bodily fluid. The bodily fluid may be blood, whole blood, blood plasma, blood serum, patient serum, urine, cerebrospinal fluid, synovial fluid or a tissue sample, for example from tumour tissue from the patient. These bodily fluids and tissue samples can be used for early detection, diagnosis, prognosis, therapy control and aftercare.

The level of a TAA, autoantibody or antigen is assayed by measuring the degree of binding between a sample and the antigen. Binding according to the invention, binding success, interactions, for example protein-protein interactions (for example protein to melanoma-specific marker sequence, such as antigen/antibody) or corresponding “means for detecting the binding success” can be visualised for example by means of fluorescence labelling, biotinylation, radio-isotope labelling or colloid gold or latex particle labelling in the conventional manner. Bound antibodies are detected with the aid of secondary antibodies, which are labelled using commercially available reporter molecules (for example Cy, Alexa, Dyomics, FITC or similar fluorescent dyes, colloidal gold or latex particles), or with reporter enzymes, such as alkaline phosphatase, horseradish peroxidase, etc. and the corresponding colorimetric, fluorescent or chemoluminescent substrates. A readout is performed for example by means of a microarray laser scanner, a CCD camera or visually.

Comparison may be performed by any number of statistical analyses, such as those described in Example 5 herein.

In another aspect is provided a method of identifying a tumor-associated antigen (TAA) as a marker for melanoma overall survival (MOS) or melanoma disease control rate (MDCR). A first group of patients with melanoma is selected who have statistically greater MOS or MDCR than a second group of patients with melanoma. The level of an autoantibody to the antigen in a sample from each of the patients in the first group is assayed. The level of the autoantibody to the antigen in each of the patients in the first group is compared to the level of the autoantibody in each of the patients in the second group. An antigen is determined to be a TAA marker for MOS or MDCR if the level of the autoantibody to the antigen is statistically different between the first group of patients and the second group of patients.

In some embodiments, the antigen is encoded by a gene listed in Table 3. In some embodiments, the TAA marker for MOS or MDCR is encoded by a gene listed in Table 3. In some embodiments, the assaying comprises b1) contacting a portion of serum from the patient with a sample of an antigen immobilized onto a solid support. In some embodiments, the solid support is a bead. In some embodiments, bead is a microsphere.

In another aspect is provided a method of identifying and treating a melanoma patient susceptible to an immune-related adverse event (irAE) after treatment with a checkpoint inhibitor. The level of one or more antigens encoded by a gene listed in Table 4 having a positive value for SAM Fold.Change is determined. The level of one or more antigens in a sample from a melanoma patient is assayed. The level of the one or more antigens is compared with an average level of the one or more antigens for a group of patients with melanoma. The checkpoint inhibitor is administered to the melanoma patient if:
(a) the level of the one or more antigens encoded by a gene listed in Table 4 having a value for SAM Fold.Change>1 in the patient is less than the average level of the one or more antigens encoded by a gene listed in Table 4 having a value for SAM Fold.Change>1 in the group of patients with melanoma, or
(b) the level of the one or more antigens encoded by a gene listed in Table 4 having a value for SAM Fold.Change<=1 in the patient is greater than the average level of the one or more antigens encoded by a gene listed in Table 4 having a value for SAM Fold.Change<=1 in the group of patients with melanoma.

In some embodiments, the antigen encoded by a gene listed in Table 4 is SDCBP or ATG4D.

In some embodiments, the number of the one or more antigens in (a) or the number of the one or more antigens in (b) exceeds 2. In some embodiments, the patient further has a reduced level of one or more antigens encoded by a gene listed in Table 4 having a value for SAM Fold.Change>1 as compared to the level in the group of patients with melanoma. In some embodiments, the assaying comprises b1) contacting a portion of serum from the patient with a sample of an antigen immobilized onto a solid support. In some embodiments, the solid support is a bead. In some embodiments, the bead is a microsphere. In some embodiments, the checkpoint inhibitor is Ipilimumab. In some embodiments, the checkpoint inhibitor is nivolumumab. In some embodiments, the checkpoint inhibitor is pembrolizumab.

In another aspect is provided a method of identifying and treating a melanoma patient with a checkpoint inhibitor. The level of one or more antigens encoded by a gene listed in Table 5 having a value for SAMR Fold.Change>1 is determined. The level of the one or more antigens is compared with an average level of the one or more antigens for a group of patients with melanoma. The checkpoint inhibitor is administered if the level of the one or more antigens in the patient is greater than the average level of the one or more antigens in the group of patients with melanoma.

In some embodiments, the antigen encoded by a gene listed in Table 5 is GPHN.

In some embodiments, the number of the one or more antigens exceeds 2. In some embodiments, the patient further has an increased level of one or more antigens encoded by a gene listed in Table 5 having a positive value R-value PFS or R-value OS as compared to the level in the group of patients with melanoma. In some embodiments, the assaying comprises contacting a portion of serum from the patient with a sample of an antigen immobilized onto a solid support. In some embodiments, the solid support is a bead. In some embodiments, the bead is a microsphere. In some embodiments, the checkpoint inhibitor is Ipilimumab. In some embodiments, the checkpoint inhibitor is nivolumumab. In some embodiments, the checkpoint inhibitor is pembrolizumab.

In another aspect is provided a method of identifying and treating a melanoma patient with a CTLA-4 inhibitor. The level of one or more antigens encoded by a gene listed in Table 6 having a value for SAMR Fold.Change>1 is determined. The level of the one or more antigens is compared with an average level of the one or more antigens for a group patients with melanoma. The CTLA-4 inhibitor is administered if the level of the one or more antigens in the patient is less than the average level of the one or more antigens in the group of patients with melanoma.

In some embodiments, the antigen encoded by a gene listed in Table 6 is SUMO2 or ATG4D.

In some embodiments, the number of the one or more antigens exceeds 2. In some embodiments, the assaying comprises contacting a portion of serum from the patient with a sample of an antigen immobilized onto a solid support. In some embodiments, the solid support is a bead. In some embodiments, the bead is a microsphere.

In another aspect is provided a method of identifying and treating a melanoma patient with a CTLA-4 inhibitor. The level of one or more antigens encoded by a gene listed in Table 6 having a value for SAMR Fold.Change<=1 is determined. The level of the one or more antigens is compared with an average level of the one or more antigens for a group of patients with melanoma. The CTLA-4 inhibitor is administered if the level of the one or more antigens in the patient is greater than the average level of the one or more antigens in the group of patients with melanoma.

In some embodiments, the number of the one or more antigens exceeds 2. In some embodiments, the assaying comprises contacting a portion of serum from the patient with a sample of an antigen immobilized onto a solid support. In some embodiments, the solid support is a bead. In some embodiments, the bead is a microsphere.

In another aspect is provided a method of identifying and treating a melanoma patient with a PD-1/PD-L1 pathway inhibitor. The level of one or more antigens encoded by a gene listed in Table 7 having a value for SAMR Fold.Change>1 is determined. The level of the one or more antigens is compared with an average level of the one or more antigens for a group of patients with melanoma. The PD-1/PD-L1 pathway inhibitor is administered if the level of the one or more antigens in the patient is greater than the average level of the one or more antigens in the group of patients with melanoma.

In some embodiments, the antigen encoded by a gene listed in Table 7 is LAMC1 or FGA.

In some embodiments, the number of the one or more antigens exceeds 2. In some embodiments, the patient further has an increased level of one or more antigens encoded by a gene listed in Table 7 having a positive value R-value PFS, R-value OS or R-value DCR as compared to the level in the group of patients with melanoma. In some embodiments, the assaying comprises contacting a portion of serum from the patient with a sample of an antigen immobilized onto a solid support. In some embodiments, the solid support is a bead. In some embodiments, the bead is a microsphere. In some embodiments, the PD-1/PD-L1 pathway inhibitor is pembrolizumab.

In another aspect is provided a method of identifying and treating a melanoma patient with pembrolizumab. The level of one or more antigens encoded by a gene listed in Table 8 having a value for SAMR Fold.Change<=1 is determined. The level of the one or more antigens is compared with an average level of the one or more antigens for a group of patients with melanoma. The pembrolizumab is administered if the level of the one or more antigens in the patient is greater than the average level of the one or more antigens in the group of patients with melanoma.

In some embodiments, the number of the one or more antigens exceeds 2. In some embodiments, the assaying comprises contacting a portion of serum from the patient with a sample of an antigen immobilized onto a solid support. In some embodiments, the solid support is a bead. In some embodiments, the bead is a microsphere.

In another aspect is provided a method of identifying and treating a melanoma patient with pembrolizumab. The level of one or more antigens encoded by a gene listed in Table 8 having a value for SAMR Fold.Change>1 is determined. The level of the one or more antigens is compared with an average level of the one or more antigens for a group of patients with melanoma. The pembrolizumab is administered if the level of the one or more antigens in the patient is less than the average level of the one or more antigens in the group of patients with melanoma.

In some embodiments, the antigen encoded by a gene listed in Table 8 is MITF, KRT7, FN1, CTSW, MIF, or SPA17.

In some embodiments, the number of the one or more antigens exceeds 2. In some embodiments, the assaying comprises contacting a portion of serum from the patient with a sample of an antigen immobilized onto a solid support. In some embodiments, the solid support is a bead. In some embodiments, the bead is a microsphere.

In another aspect is provided a method of identifying an antigen predictive of development of an irAE or colitis in a patient with melanoma who is treated with a checkpoint inhibitor comprising: a) selecting a first group of patients with melanoma who developed irAE or colitis after treatment with the checkpoint inhibitor, b) assaying the level of an autoantibody to an antigen in a sample from a patient in the first group who developed irAE or colitis, c) comparing the level of the autoantibody from the patient in the first group to the level of the autoantibody in a second group of patients with melanoma after treatment with the checkpoint inhibitor but did not develop irAE or colitis, d) determining that the antigen is predictive of development of an irAE or colitis if the level of the autoantibody to the antigen is statistically different between the first group of patients and the second group of patients.

In some embodiments, the antigen is an antigen encoded by a gene selected from SUMO2, MAGED2, PIAS3, MITF, GRP, AP2B1, PRKCI, BTBD2, AKT2, UBE2Z, L1CAM, LAMC1, GABARAPL2, RPLP0, SDCBP, AP1S1, CFB, FGA, IL3, IL4R, AMPH, LEPR, TP53, GPHN, IL23A, BAG6, BICD2, TMEM98, KDM4A, UBTF, CASP8, PCDH1, RELT, SPTBN1, MUM1, RPLP2, KRT7, FN1, MAGEB4, CTSW, NCOA1, MIF, SPA17, FGFR1, KRT19, TPM2, and ATG4D.

In some embodiments, the number of the one or more antigens exceeds 2. In some embodiments, the assaying comprises contacting a portion of serum from the patient with a sample of an antigen immobilized onto a solid support. In some embodiments, the solid support is a bead. In some embodiments, the bead is a microsphere.

In another aspect is provided a method of identifying an antigen predictive of development of colitis in a patient with melanoma who is treated with a checkpoint inhibitor comprising: a) selecting a first group of patients with melanoma who developed colitis after treatment with the checkpoint inhibitor, b) assaying the level of an autoantibody to an antigen in a sample from a patient in the first group who developed colitis, c) comparing the level of the autoantibody from the patient in the first group to the level of the autoantibody in a second group of patients with melanoma after treatment with the checkpoint inhibitor but did not develop colitis, d) determining that the antigen is predictive of development of colitis if the level of the autoantibody to the antigen is statistically different between the first group of patients and the second group of patients.

In some embodiments, the antigen is an antigen encoded by a gene selected from SUMO2, MAGED2, PIAS3, MITF, GRP, AP2B1, PRKCI, BTBD2, AKT2, UBE2Z, L1CAM, LAMC1, GABARAPL2, RPLP0, SDCBP, AP1S1, CFB, FGA, IL3, IL4R, AMPH, LEPR, TP53, GPHN, IL23A, BAG6, BICD2, TMEM98, KDM4A, UBTF, CASP8, PCDH1, RELT, SPTBN1, MUM1, RPLP2, KRT7, FN1, MAGEB4, CTSW, NCOA1, MIF, SPA17, FGFR1, KRT19, TPM2, and ATG4D.

In some embodiments, the number of the one or more antigens exceeds 2. In some embodiments, the assaying comprises contacting a portion of serum from the patient with a sample of an antigen immobilized onto a solid support. In some embodiments, the solid support is a bead. In some embodiments, the bead is a microsphere.

In another aspect is provided a method of identifying an antigen predictive of development of colitis in a patient with melanoma who is treated with a checkpoint inhibitor comprising: a) selecting a first group of patients with melanoma who developed colitis after treatment with the checkpoint inhibitor, b) assaying the level of an autoantibody to an antigen in a sample from a patient in the first group who developed colitis, c) comparing the level of the autoantibody from the patient in the first group to the level of the autoantibody in a second group of patients with melanoma after treatment with the checkpoint inhibitor but did not develop colitis, d) determining that the antigen is predictive of development of colitis if the level of the autoantibody to the antigen is statistically different between the first group of patients and the second group of patients.

In some embodiments, the antigen is an antigen encoded by a gene selected from SUMO2, MAGED2, PIAS3, MITF, GRP, AP2B1, PRKCI, BTBD2, AKT2, UBE2Z, L1CAM, LAMC1, GABARAPL2, RPLP0, SDCBP, AP1S1, CFB, FGA, IL3, IL4R, AMPH, LEPR, TP53, GPHN, IL23A, BAG6, BICD2, TMEM98, KDM4A, UBTF, CASP8, PCDH1, RELT, SPTBN1, MUM1.

In some embodiments, the antigen is an antigen encoded by a gene selected from SUMO2, MAGED2, PIAS3, MITF, GRP, AP2B1, PRKCI, BTBD2, AKT2, UBE2Z, L1CAM, LAMC1, GABARAPL2, RPLP0, SDCBP, AP1S1, CFB, FGA, IL3, IL4R, AMPH, LEPR, TP53, GPHN, IL23A, BAG6, BICD2, TMEM98, KDM4A, UBTF, CASP8, PCDH1, RELT, SPTBN1, MUM1.

In some embodiments, the checkpoint inhibitor is ipilumab and the antigen is an antigen encoded by a gene selected from MAGED2, PIAS3, MITF, PRKC1, and A2B1. In some embodiments, the checkpoint inhibitor is ipilumab and the antigen is an antigen encoded by a gene selected from AKT2, AP1S1, AP2B1, BAG6, BICD2, BTBD2, CASP8, CFB, FGA, GABARAPL2, GPHN, GRP, IL23A, IL3, IL4R, KDM4A, L1CAM, LAMC1, MAGED2, MITF, PCDH1, PIAS3, PRKCI, RELT, SDCBP, SPTBN1, SUMO2, TMEM98, UBE2Z, and UBTF. In some embodiments, the checkpoint inhibitor is ipilumab and the antigen is an antigen encoded by a gene selected from UBE2Z, L1CAM, GABARAPL2, CFB, IL3, RELT, FGA, and IL4R.

In some embodiments, the checkpoint inhibitor is a combination of ipulimuab and nivolumab and the antigen is an antigen encoded by a gene selected from PIAS3, SUMO2, MITF, GRP, PRKCI, AP2B1, SDCBP, PDCH1, SPTBN1, and UBTF. In some embodiments, the antigen is predictive of an increased risk of development of colitis and is encoded by a gene selected from RELT, CASP8, UBE2Z, IL4R, LAMC1, L1CAM, MITF. In some embodiments, the antigen is predictive of a decreased risk of development of colitis and is encoded by a gene selected from SUMO2, GRP, MIF.

In some embodiments, the number of the one or more antigens exceeds 2. In some embodiments, the assaying comprises contacting a portion of serum from the patient with a sample of an antigen immobilized onto a solid support. In some embodiments, the solid support is a bead. In some embodiments, the bead is a microsphere.

In another aspect is provided a method of identifying an antigen predictive of development of an irAE in a patient with melanoma who is treated with a checkpoint inhibitor comprising: a) selecting a first group of patients with melanoma who developed irAE after treatment with the checkpoint inhibitor, b) assaying the level of an autoantibody to an antigen in a sample from a patient in the first group who developed the irAE, c) comparing the level of the autoantibody from the patient in the first group to the level of the autoantibody in a second group of patients with melanoma after treatment with the checkpoint inhibitor but did not develop the irAE, d) determining that the antigen is predictive of development of the irAE if the level of the autoantibody to the antigen is statistically different between the first group of patients and the second group of patients.

In some embodiments, the antigen is predictive of an increased risk of development of the irAE and is encoded by a gene selected from IL4R, L1CAM, MITF, PIAS3, AP1S1, ATG4D, and RPLP2.

In some embodiments, the antigen is predictive of a decreased risk of development of the irAE and is encoded by a gene selected from MIF, NCOA1, FGFR1, and SDCBP.

In some embodiments, the checkpoint inhibitor is ipilumab and wherein the antigen is an antigen encoded by a gene selected from MAGED2, PIAS3, MITF, PRKC1, and A2B1.

In some embodiments, the number of the one or more antigens exceeds 2. In some embodiments, the assaying comprises contacting a portion of serum from the patient with a sample of an antigen immobilized onto a solid support. In some embodiments, the solid support is a bead. In some embodiments, the bead is a microsphere.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Production of Recombinant Autoantigens

Recombinant antigens were produced in Escherichia coli. Five cDNA libraries originating from different human tissues (fetal brain, colon, lung, liver, CD4 induced and non-induced T cells) were used for the recombinant production of human antigens. All of these cDNA libraries were oligo(dT)-primed, containing the coding region for an N-terminally located hexa-histidine-tag and were under transcriptional control of the lactose inducible promoter from E. coli]. Sequence integrity of the cDNA libraries was confirmed by 5′ DNA sequencing. Additionally, expression clones representing the full-length sequence derived from the human ORFeome collection were included. Individual antigens were designed in silico, synthesized chemically (Life Technologies, Carlsbad, USA) and cloned into the expression vector pQE30-NST fused to the coding region for the N-terminal-located His6-tag. Of the antigens. Recombinant gene expression was performed in E. coli SCS1 cells carrying plasmid pSE111 for improved expression of human genes. Cells were cultivated in 200 ml auto-induction medium (Overnight Express auto-induction medium, Merck, Darmstadt, Germany) overnight and harvested by centrifugation. Bacterial pellets were lysed by resuspension in 15 ml lysis buffer (6 M guanidinium-HCl, 0.1 M NaH2PO4, 0.01 M Tris-HCl, pH 8.0).

Soluble proteins were affinity-purified after binding to Protino® Ni-IDA 1000 Funnel Column (Macherey-Nagel, Düdren, Germany). Columns were washed with 8 ml washing buffer (8 M urea, 0.1 M NaH2PO4, 0.01 M Tris-HCl, pH 6.3). Proteins were eluted in 3 ml elution buffer (6 M urea, 0.1 M NaH2PO4, 0.01 M Tris-HCl, 0.5% (w/v) trehalose pH 4.5). Each protein preparation was transferred into 2D-barcoded tubes, lyophilized and stored at −20° C.

### Example 2: Selection of Antigens and Design of the Cancer Screen

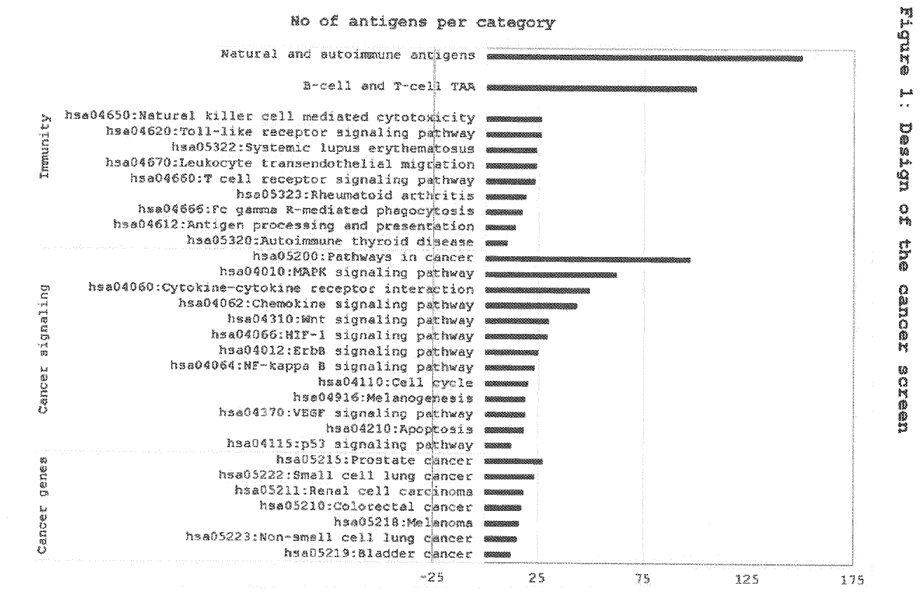

A bead-based array was designed to screen for autoantibodies binding to tumor-associated antigens (TAA), proteins expressed from mutated or overexpressed cancer genes, and proteins playing a role in cancer signaling pathways. Furthermore, self-reactive antigens of normal humans and typical autoimmune antigens were included. In total, 842 potential antigens were selected. FIG. 1 shows the number of screening antigens per category.

### Example 3: Coupling of Antigens to Beads

For the production of bead-based arrays (BBA), the proteins were coupled to magnetic carboxylated color-coded beads (MagPlex™ microspheres, Luminex Corporation, Austin, Tex., USA). The manufacturer's protocol for coupling proteins to MagPlex™ microspheres was adapted to use liquid handling systems. A semi-automated coupling procedure of one BBA encompassed 384 single, separate coupling reactions, which were carried out in four 96-well plates. For each single coupling reaction, up to 12.5 μg antigen and 8.8×105 MagPlex™ beads of one color region (ID) were used. All liquid handling steps were carried out by either an eight-channel pipetting system (Starlet, Hamilton Robotics, Bonaduz, Switzerland) or a 96-channel pipetting system (Evo Freedom 150, Tecan, Mannderdorf, Switzerland). For semi-automated coupling, antigens were dissolved in H2O, and aliquots of 60 microliters were transferred from 2D barcode tubes to 96-well plates. MagPlex™ microspheres were homogeneously resuspended and each bead ID was transferred in one well of a 96-well plate. The 96-well plates containing the microspheres were placed on a magnetic separator (LifeSep™, Dexter Magnetic Technologies Inc., Elk Grove Village, USA) to sediment the beads for washing steps and on a microtiter plate shaker (MTS2/4, IKA) to facilitate permanent mixing for incubation steps.

For coupling, the microspheres were washed three times with activation buffer (100 mM NaH2PO4, pH 6.2) and resuspended in 120 μl activation buffer. To obtain reactive sulfo-NHS-ester intermediates, 15 μl 1-ethly-3-(3-dimethlyaminopropyl) carbodiimide (50 mg/ml) and 15 μl N-hydroxy-succinimide (50 mg/ml) were applied to microspheres. After 20 minutes incubation (900 rpm, room temperature (RT)) the microspheres were washed three times with coupling buffer (50 mM MES, pH 5.0) and resuspended in 65 μl coupling buffer. Immediately, 60 μl antigen solution was added to reactive microspheres and coupling took place over 120 minutes under permanent mixing (900 rpm, RT). After three wash cycles using washing buffer (PBS, 0.1% Tween20) coupled beads were resuspended in blocking buffer (PBS, 1% BSA, 0.05% ProClin300), incubated for 20 minutes (900 rpm, RT) and then transferred to be maintained at 4-8° C. for 12-72 h.

Finally, a multiplex BBA was produced by pooling 384 antigen-coupled beads.

### Example 4: Incubation of Serum Samples with Antigen-Coupled Beads

Serum samples were transferred to 2D barcode tubes and a 1:100 serum dilution was prepared with assay buffer (PBS, 0.5% BSA, 10% E. coli lysate, 50% Low-Cross buffer (Candor Technologies, Nürnberg, Germany)) in 96-well plates. The serum dilutions were first incubated for 20 minutes to neutralize any human IgG eventually directed against E. coli proteins. The BBA was sonicated for 5 minutes and the bead mix was distributed in 96-well plates. After three wash cycles with washing buffer (PBS, 0.05% Tween20) serum dilutions (50 μl) were added to the bead mix and incubated for 20 h (900 rpm, 4-8° C.). Supernatants were removed from the beads by three wash cycles, and secondary R-phycoerythrin-labeled antibody (5 μg/ml, goat anti-human, Dianova, Hamburg, Germany) was added for a final incubation of 45 minutes (900 rpm, RT). The beads were washed three times with washing buffer (PBS, 0.1% Tween20) and resuspended in 100 μl sheath fluid (Luminex Corporation). Subsequently, beads were analyzed in a FlexMap3D device for fluorescent signal readout (DD gate 7.500-15.000; sample size: 80 μl; 1000 events per bead ID; timeout 60 sec). The binding events were displayed as median fluorescence intensity (MFI). Measurements were disregarded when low numbers of bead events (<30 beads) were counted per bead ID.

### Example 5: Statistical Analysis

Statistical analysis was performed to identify biomarkers associated with the effectiveness and side effects of immune-oncology therapy. Autoantibody levels were correlated with overall survival (OS), progression-free survival (OS), and irAE using Spearman's rank correlation test. In the case, when two groups were compared the permutation based statistical technique Significance of microarrays in the R-programming language (SAMR) was used (Tusher et al., 2001). The strength of differences between the two test groups is computed as SAMR score_d. Furthermore, receiver-operating characteristics were calculated to provide area under the curve (AUC) values for each antigen. The ROC curves were generated using the package pROC (Robin et al., 2011).

To evaluate the tumor response to treatment, the best overall response (BOR) was determined by RECIST v1.1 criteria and the disease control rate was calculated. The disease control rate (DCR) is the percentage of patients achieving complete response (CR), or partial response (PR) or stable disease (SD). To identify biomarkers that predict clinical response in in pre-treatment samples (T0), a responder was defined as with CR, PR, or SD and autoantibody profiles of patients with DCR compared to patients with progressive disease.

### Example 6: Collection of Serum Samples from Patients with Metastatic Melanoma Treated with Different Immune Checkpoint Inhibitors

Serum samples of metastatic melanoma patients treated with immune checkpoint inhibitors were collected at the National Center for Tumor Diseases (NCT, Heidelberg, Germany). Serum samples were collected prior to immune checkpoint inhibitor treatment (T0, baseline or pre-treatment sample) and at two time points during treatment (post-treatment samples). The T1 corresponds to 90 days (3 month) and the T2 samples corresponds to 180 days (6 month).

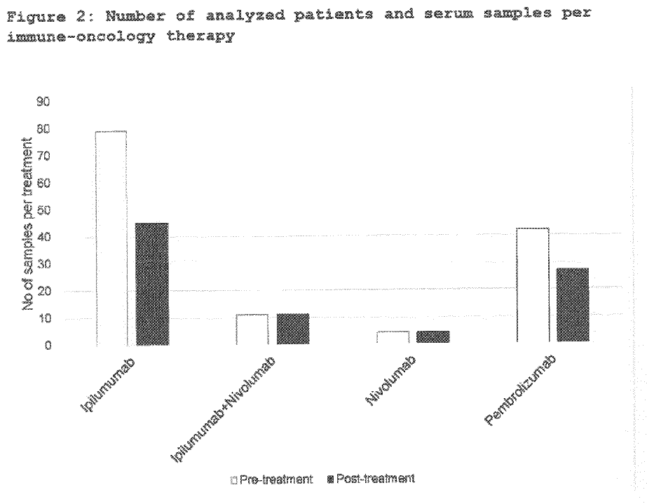

FIG. 2 shows the number of patients and samples per treatment group.

Patient data were provided on a standardized form including demographics (age, gender), the type of checkpoint inhibitor treatment, the date of therapy start, and best response according to “Response Evaluation Criteria in Solid tumors” (RECIST 1.1. criteria), graded into complete response (CR), partial response (PR), stable disease (SD), and progressive disease (PD) (Eisenhauer et al., 2009).

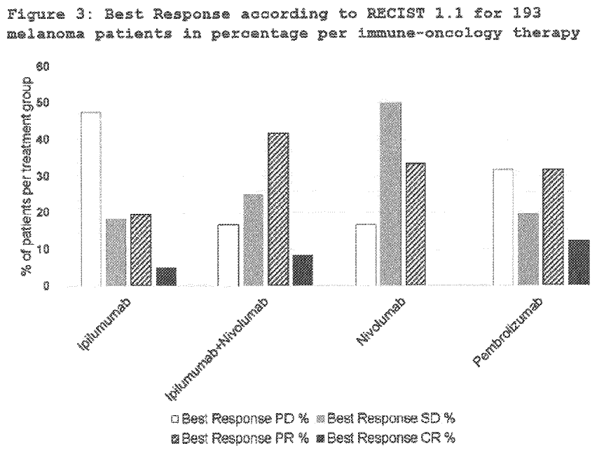

FIG. 3 shows the response categories (CR, PR, SD, and PD) achieved by patients treated with different checkpoint inhibitors.

Furthermore, details on immune-related adverse events (irAE) were recorded.

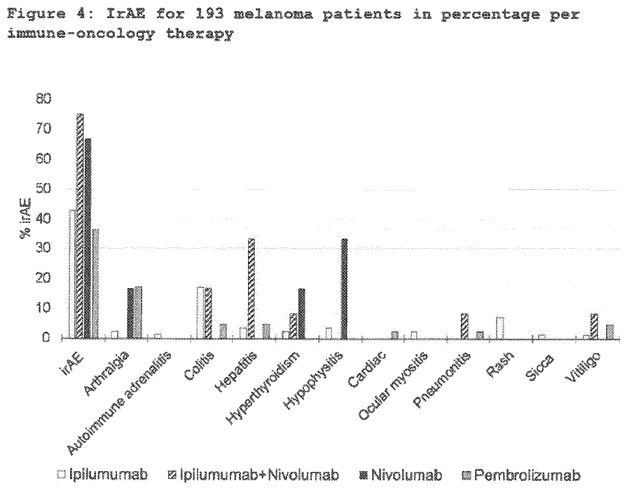

FIG. 4 shows the different irAE, which occurred following treatment with different checkpoint inhibitors. The highest percentage (75%) of irAE occurred during ipilimumab/nivolumab combination therapy. Colitis most frequently occurred during ipiliumab and ipilimumab/nivolumab combination therapy.

The survival time (overall survival, OS) was calculated as the time from start of treatment to death or the last contact date.

Progression-free survival (PFS) was calculate as the time from start of treatment to progression. When progression was not observed the time from start to death or last visit was calculated.

### Example 7: Characterization of the Autoantibody Response in Melanoma Patients

The presence of a tumor can induce a humoral immune response to tumor-associated antigens (TAA) and self-antigens. This autoantibody response may be utilized to characterize the immune-status of a cancer patients receiving immune-oncology therapy. Pre- and post-treatment serum samples from 193 melanoma patients treated with anti-CTLA-4 (ipilumumab), anti-PD-1 (nivolumab or pembrolizumab) or anti-CTLA-4/anti-PD-1 combination therapy were analyzed for the presence of autoantibodies directed towards 842 preselected tumor-associated antigens (TAA) and self-antigens.

Table 1 shows the autoantibody response of melanoma patients against 135 antigens. Markers correlating with different clinical endpoints are extracted and shown in separate tables (T). Table 1 includes the following antigens:

GRAMD4, TEX264, CREB3L1, NCBP3/C17orf85, FRS2, S100A8, TRAF3IP3, NOVA2, C15orf48; NMES1, MIF, CTAG1B, CAP2, CSNK2A1, IGF2BP2, GPHN, SDCBP, HSPA1B, SPTB, HES1, MMP3, PAPOLG, SNRPD1, SSB, XRCC5, XRCC6, EOMES, ERBB3, ATG4D, ELMO2, AKAP13, HSPA2, SMAD9, BIRC5, FGA, PDCD6IP, RPS6KA1, USB1, BCL7B, EIF3E, CENPH, GNG12, CCDC51, HUS1, HSPB1, KLKB1, LARP1, LGALS3BP, OGT, PECAM1, NRIP1, PPP1R2, IL36RN, RALY, S100A14, SNRNP70, SNRPA, MUC12, HIST2H2AA3, SIVA1, AQP4, RPLP2, SDC1, TRA2B, EGLN2, RAPGEF3, RPRM, NSD3/WHSC1L1, ATP13A2, CTSW, CXXC1, FADD, ACTB, MLLT6, ARRB1, CEACAM5, GSK3A, HDAC1, LAMC1, MSH2, MAZ, PTPRR, DFFA, DHFR, FLNA, CCNB1, SHC1, CALR, GRK6, GNAI2, FGFR1, CENPV, CEP131, PPP1R12A, CASP10, FOXO1, CPSF1, GRK2, AKT3, ANXA4, ATP1B3, BCR, CDR2L, NME1, CXCL13, CXCL5, DNAJC8, DUSP3, EEF2, MAGED1, EIF4E2, HSPD1, IL17A, MAPT, POLR3B, SIPA1L1, SUMO2, TRIP4, UBAP1, BTRC, EGFR, FN1, KRT7, LAMB2, MITF, PPL, SIGIRR, SPA17, SUFU, TOLLIP, TONSL, PLIN2, RFWD2, ABCB8, SQSTM1, and CTAG2.

TRA2B was tested as a post-translationally modified protein, in which the amino acid arginine was modified by citrullination or deamination into the amino acid citrulline.

The modified protein is referred to as “TRA2B_cit”. Autoantibodies binding to citrullinated antigens or peptides (ACPA) are found in rheumatoid arthritis (RA).

TABLE 1
List of all identified antigens

| | | Gene Symbol | | | | | | | | |
|---|---|---|---|---|---|---|---|---|---|---|
| | | and Exemplary | | | | | | | | |
| | Gene | Antigen | | T | T | T | T | T | T | T |
| ID | ID | Sequence | Gene Name | 2 | 3 | 4 | 5 | 6 | 7 | 8 |
| 1 | 23151 | GRAMD4 (SEQ | GRAM domain | | x | | x | | | |
| | | ID NO: 1) | containing 4 | | | | | | | |
| 2 | 51368 | TEX264 (SEQ | testis expressed | x | | x | | | x | x |
| | | ID NO: 2) | 264 | | | | | | | |
| 3 | 90993 | CREB3L1 (SEQ | cAMP responsive | x | | x | | x | | |
| | | ID NO: 3) | element binding | | | | | | | |
| | | | protein 3-like 1 | | | | | | | |
| 4 | 55421 | NCBP3; | nuclear cap binding | | | | | | x | |
| | | C17orf85 (SEQ | subunit 3 | | | | | | | |
| | | ID NO: 4) | | | | | | | | |
| 5 | 10818 | FRS2 (SEQ ID | fibroblast growth | | | x | x | x | | |
| | | NO: 5) | factor receptor | | | | | | | |
| | | | substrate 2 | | | | | | | |
| 6 | 6279 | S100A8 (SEQ ID | S100 calcium | | | x | | | x | x |
| | | NO: 6) | binding protein A8 | | | | | | | |
| 7 | 80342 | TRAF3IP3 (SEQ | TRAF3 interacting | | | | | | x | |
| | | ID NO: 7) | protein 3,- | | | | | | | |
| 8 | 4858 | NOVA2 (SEQ ID | neuro-oncological | x | | | | | x | |
| | | NO: 8) | ventral antigen 2 | | | | | | | |
| 9 | 84419 | C15orf48; | chromosome 15 open | x | x | | | | x | |
| | | NMES1 (SEQ ID | reading frame 48 | | | | | | | |
| | | NO: 9) | | | | | | | | |
| 10 | 4282 | MIF (SEQ ID | macrophage | x | x | | | | | x |
| | | NO: 10) | migration | | | | | | | |
| | | | inhibitory factor | | | | | | | |
| | | | (glycosylation- | | | | | | | |
| | | | inhibiting factor) | | | | | | | |
| 11 | 1485 | CTAG1B (SEQ ID | cancer/testis | x | x | | | | | |
| | | NO: 11) | antigen 1B | | | | | | | |
| 12 | 10486 | CAP2 (SEQ ID | CAP, adenylate | x | | x | | | | x |
| | | NO: 12) | cyclase-associated | | | | | | | |
| | | | protein, 2 (yeast) | | | | | | | |
| 13 | 1457 | CSNK2A1 (SEQ | casein kinase 2, | x | | | | | x | |
| | | ID NO: 13) | alpha 1 polypeptide | | | | | | | |
| 14 | 10644 | IGF2BP2 (SEQ | insulin-like growth | | x | | | | x | |
| | | ID NO: 14) | factor 2 mRNA | | | | | | | |
| | | | binding protein 2 | | | | | | | |
| 15 | 10243 | GPHN (SEQ ID | gephyrin | x | x | | x | | | |
| | | NO: 15) | | | | | | | | |
| 16 | 6386 | SDCBP (SEQ ID | syndecan binding | | | x | | x | | |
| | | NO: 16) | protein (syntenin) | | | | | | | |
| 17 | 3304 | HSPA1B (SEQ ID | heat shock 70 kDa | | | x | | | | x |
| | | NO: 17) | protein 1B | | | | | | | |
| 18 | 6710 | SPTB (SEQ ID | spectrin, beta, | | | x | | | | x |
| | | NO: 18) | erythrocytic | | | | | | | |
| 19 | 3280 | HES1 (SEQ ID | hes family bHLH | | | | | | x | |
| | | NO: 19) | transcription | | | | | | | |
| | | | factor 1 | | | | | | | |
| 20 | 4314 | MMP3 (SEQ ID | stromelysin 1 | | | | | | x | |
| | | NO: 20) | | | | | | | | |
| 21 | 64895 | PAPOLG (SEQ ID | poly(A) polymerase | | x | | x | | | |
| | | NO: 21) | gamma | | | | | | | |
| 22 | 6632 | SNRPD1 (SEQ ID | small nuclear | | | | | | x | |
| | | NO: 22) | ribonucleoprotein | | | | | | | |
| | | | D1 polypeptide | | | | | | | |
| 23 | 6741 | SSB (SEQ ID | Sjogren syndrome | | | | | | x | |
| | | NO: 23) | antigen B | | | | | | | |
| 24 | 7520 | XRCC5 (SEQ ID | X-ray repair cross | | | | | x | x | |
| | | NO: 24) | complementing 5 | | | | | | | |
| 25 | 2547 | XRCC6 (SEQ ID | X-ray repair cross | | | | | x | x | |
| | | NO: 25) | complementing 6 | | | | | | | |
| 26 | 8320 | EOMES (SEQ ID | eomesodermin | | | | | x | x | |
| | | NO: 26) | | | | | | | | |
| 27 | 2065 | ERBB3 (SEQ ID | erb-b2 receptor | x | | x | | | | x |
| | | NO: 27) | tyrosine kinase 3 | | | | | | | |
| 28 | 84971 | ATG4D (SEQ ID | autophagy related | | | x | | x | | |
| | | NO: 28) | 4D, cysteine | | | | | | | |
| | | | peptidase | | | | | | | |
| 29 | 63916 | ELMO2 (SEQ ID | engulfment and cell | | | | | x | | x |
| | | NO: 29) | motility 2 | | | | | | | |
| 30 | 11214 | AKAP13 (SEQ ID | A kinase (PRKA) | x | | | | | | |
| | | NO: 30) | anchor protein 13 | | | | | | | |
| 31 | 3306 | HSPA2 (SEQ ID | heat shock 70 kDa | | | x | | | | |
| | | NO: 31) | protein 2 | | | | | | | |
| 32 | 4093 | SMAD9 (SEQ ID | SMAD family member | | | x | | | | |
| | | NO: 32) | 9 | | | | | | | |
| 33 | 332 | BIRC5 (SEQ ID | baculoviral IAP | | | | x | | | |
| | | NO: 33) | repeat containing 5 | | | | | | | |
| 34 | 2243 | FGA (SEQ ID | fibrinogen alpha | | | | | | x | |
| | | NO: 34) | chain | | | | | | | |
| 35 | 10015 | PDCD6IP (SEQ | programmed cell | | | | | | | x |
| | | ID NO: 35) | death 6 interacting | | | | | | | |
| | | | protein | | | | | | | |
| 36 | 6195 | RPS6KA1 (SEQ | ribosomal protein | | | | x | | | |
| | | ID NO: 36) | S6 kinase, 90 kDa, | | | | | | | |
| | | | polypeptide 1 | | | | | | | |
| 37 | 79650 | USB1 (SEQ ID | U6 snRNA biogenesis | x | | | x | | | |
| | | NO: 37) | 1 | | | | | | | |
| 38 | 9275 | BCL7B (SEQ ID | B-cell CLL/lymphoma | | | | x | | | |
| | | NO: 38) | 7B | | | | | | | |
| 39 | 3646 | EIF3E (SEQ ID | eukaryotic | | | | x | | | |
| | | NO: 39) | translation | | | | | | | |
| | | | initiation factor | | | | | | | |
| | | | 3, subunit E | | | | | | | |
| 40 | 64946 | CENPH (SEQ ID | centromere protein | x | | | x | | | |
| | | NO: 40) | H | | | | | | | |
| 41 | 55970 | GNG12 (SEQ ID | guanine nucleotide | | | | x | | | |
| | | NO: 41) | binding protein (G | | | | | | | |
| | | | protein), gamma 12 | | | | | | | |
| 42 | 79714 | CCDC51 (SEQ ID | coiled-coil domain | | | | x | | | |
| | | NO: 42) | containing 51 | | | | | | | |
| 43 | 3364 | HUS1 (SEQ ID | HUS1 checkpoint | | | | x | | | |
| | | NO: 43) | homolog (S. pombe) | | | | | | | |
| 44 | 3315 | HSPB1 (SEQ ID | heat shock 27 kDa | | | | | | | x |
| | | NO: 44) | protein 1 | | | | | | | |
| 45 | 3818 | KLKB1 (SEQ ID | kallikrein B, | | | | | | | x |
| | | NO: 45) | plasma (Fletcher | | | | | | | |
| | | | factor) 1 | | | | | | | |
| 46 | 23367 | LARP1 (SEQ ID | La | | | | | | | x |
| | | NO: 46) | ribonucleoprotein | | | | | | | |
| | | | domain family, | | | | | | | |
| | | | member 1 | | | | | | | |
| 47 | 3959 | LGALS3BP (SEQ | lectin, | | | | | | | x |
| | | ID NO: 47) | galactoside- | | | | | | | |
| | | | binding, soluble, 3 | | | | | | | |
| | | | binding protein | | | | | | | |
| 48 | 8473 | OGT (SEQ ID | O-linked N- | | | | | | | x |
| | | NO: 48) | acetylglucosamine | | | | | | | |
| | | | (GlcNAc) | | | | | | | |
| | | | transferase | | | | | | | |
| 49 | 5175 | PECAM1 (SEQ ID | platelet/endothelia | | | | | | | x |
| | | NO: 49) | 1 cell adhesion | | | | | | | |
| | | | molecule 1 | | | | | | | |
| 50 | 8204 | NRIP1 (SEQ ID | nuclear receptor | x | | | | | x | |
| | | NO: 50) | interacting protein | | | | | | | |
| | | | 1 | | | | | | | |
| 51 | 5504 | PPP1R2 (SEQ ID | protein phosphatase | | | | | | x | |
| | | NO: 51) | 1, regulatory | | | | | | | |
| | | | (inhibitor) subunit | | | | | | | |
| | | | 2 | | | | | | | |
| 52 | 26525 | IL36RN (SEQ ID | interleukin 36 | | | | | | x | |
| | | NO: 52) | receptor antagonist | | | | | | | |
| 53 | 22913 | RALY (SEQ ID | RALY heterogeneous | | | | | | x | |
| | | NO: 53) | nuclear | | | | | | | |
| | | | ribonucleoprotein | | | | | | | |
| 54 | 57402 | S100A14 (SEQ | S100 calcium | | | | | | x | |
| | | ID NO: 54) | binding protein A14 | | | | | | | |
| 55 | 6625 | SNRNP70 (SEQ | small nuclear | | | | | | x | |
| | | ID NO: 55) | ribonucleoprotein | | | | | | | |
| | | | U1 subunit 70 | | | | | | | |
| 56 | 6626 | SNRPA (SEQ ID | small nuclear | x | | | | | x | |
| | | NO: 56) | ribonucleoprotein | | | | | | | |
| | | | polypeptide A | | | | | | | |
| 57 | 10071 | MUC12 (SEQ ID | mucin 12, cell | | | x | | | | |
| | | NO: 57) | surface associated | | | | | | | |
| 58 | 8337 | HIST2H2AA3 | histone cluster 2, | | | x | | | | |
| | | (SEQ ID NO: 58) | H2aa3 | | | | | | | |
| 59 | 10572 | SIVA1 (SEQ ID | SIVA1, apoptosis- | | x | | | | | |
| | | NO: 59) | inducing factor | | | | | | | |
| 60 | 361 | AQP4 (SEQ ID | aquaporin 4 | x | x | | | | | |
| | | NO: 60) | | | | | | | | |
| 61 | 6181 | RPLP2 (SEQ ID | ribosomal protein, | x | | | | | | |
| | | NO: 61) | large, P2 | | | | | | | |
| 62 | 6382 | SDC1 (SEQ ID | syndecan 1 | x | | | | | | |
| | | NO: 62) | | | | | | | | |
| 63 | 6434 | TRA2B_cit (SEQ | transformer 2 beta | x | | | | | | |
| | | ID NO: 63) | homolog | | | | | | | |
| | | | (Drosophila) | | | | | | | |
| 64 | 112398 | EGLN2 (SEQ ID | egl-9 family | | | | | | | x |
| | | NO: 64) | hypoxia-inducible | | | | | | | |
| | | | factor 2 | | | | | | | |
| 65 | 10411 | RAPGEF3 (SEQ | Rap guanine | | | | | | | x |
| | | ID NO: 65) | nucleotide exchange | | | | | | | |
| | | | factor (GEF) 3 | | | | | | | |
| 66 | 56475 | RPRM (SEQ ID | reprimo, TP53 | | | | | | | x |
| | | NO: 66) | dependent G2 arrest | | | | | | | |
| | | | mediator candidate | | | | | | | |
| 67 | 54904 | NSD3; | nuclear receptor | | | | | | | x |
| | | WHSC1L1 (SEQ | binding SET domain | | | | | | | |
| | | ID NO: 67) | protein 3 | | | | | | | |
| 68 | 23400 | ATP13A2 (SEQ | ATPase type 13A2 | | | | | | | x |
| | | ID NO: 68) | | | | | | | | |
| 69 | 1521 | CTSW (SEQ ID | cathepsin W | | | | | | | x |
| | | NO: 69) | | | | | | | | |
| 70 | 30827 | CXXC1 (SEQ ID | CXXC finger protein | | | | | | | x |
| | | NO: 70) | 1 | | | | | | | |
| 71 | 8772 | FADD (SEQ ID | Fas (TNFRSF6)- | | | | | | | x |
| | | NO: 71) | associated via | | | | | | | |
| | | | death domain | | | | | | | |
| 72 | 60 | ACTB (SEQ ID | actin, beta | x | | | | | x | |
| | | NO: 72) | | | | | | | | |
| 73 | 4302 | MLLT6 (SEQ ID | myeloid/lymphoid or | x | | | | | x | |
| | | NO: 73) | mixed-lineage | | | | | | | |
| | | | leukemia | | | | | | | |
| 74 | 408 | ARRB1 (SEQ ID | arrestin, beta 1 | x | | | | | x | |
| | | NO: 74) | | | | | | | | |
| 75 | 1048 | CEACAM5 (SEQ | carcinoembryonic | | | | | | x | |
| | | ID NO: 75) | antigen-related | | | | | | | |
| | | | cell adhesion | | | | | | | |
| | | | molecule 5 | | | | | | | |
| 76 | 2931 | GSK3A (SEQ ID | glycogen synthase | | | | | | x | |
| | | NO: 76) | kinase 3 alpha | | | | | | | |
| 77 | 3065 | HDAC1 (SEQ ID | histone deacetylase | | | | | | x | |
| | | NO: 77) | 1 | | | | | | | |
| 78 | 3915 | LAMC1 (SEQ ID | laminin, gamma 1 | x | | | | | x | |
| | | NO: 78) | | | | | | | | |
| 79 | 4436 | MSH2 (SEQ ID | mutS homolog 2 | | | | | | x | |
| | | NO: 79) | | | | | | | | |
| 80 | 4150 | MAZ (SEQ ID | NYC-associated zinc | | | | | | x | |
| | | NO: 80) | finger protein | | | | | | | |
| | | | (purine-binding | | | | | | | |
| | | | transcription | | | | | | | |
| | | | factor) | | | | | | | |
| 81 | 5801 | PTPRR (SEQ ID | protein tyrosine | | | | | | x | |
| | | NO: 81) | phosphatase, | | | | | | | |
| | | | receptor type, R | | | | | | | |
| 82 | 1676 | DFFA (SEQ ID | DNA fragmentation | | | | | | x | |
| | | NO: 82) | factor, 45 kDa, | | | | | | | |
| | | | alpha polypeptide | | | | | | | |
| 83 | 1719 | DHFR (SEQ ID | dihydrofolate | | | | | | x | |
| | | NO: 83) | reductase | | | | | | | |
| 84 | 2316 | FLNA (SEQ ID | filamin A, alpha | | | | | | x | |
| | | NO: 84) | | | | | | | | |
| 85 | 891 | CCNB1 (SEQ ID | cyclin B1 | | | | | | x | |
| | | NO: 85) | | | | | | | | |
| 86 | 6464 | SHC1 (SEQ ID | SHC (Src homology 2 | x | | | | | x | |
| | | NO: 86) | domain containing) | | | | | | | |
| | | | transforming | | | | | | | |
| | | | protein 1 | | | | | | | |
| 87 | 811 | CALR (SEQ ID | calreticulin | | | | | | x | |
| | | NO: 87) | | | | | | | | |
| 88 | 2870 | GRK6 (SEQ ID | G protein-coupled | x | x | | | | | |
| | | NO: 88) | receptor kinase 6 | | | | | | | |
| 89 | 2771 | GNAI2 (SEQ ID | G protein subunit | | | | | | x | |
| | | NO: 89) | alpha i2 | | | | | | | |
| 90 | 2260 | FGFR1 (SEQ ID | fibroblast growth | | x | | | | | |
| | | NO: 90) | factor receptor 1 | | | | | | | |
| 91 | 201161 | CENPV (SEQ ID | centromere protein | | | | | | x | |
| | | NO: 91) | V | | | | | | | |
| 92 | 22994 | CEP131 (SEQ ID | centrosomal protein | | | | | | x | |
| | | NO: 92) | 131 kDa | | | | | | | |
| 93 | 4659 | PPP1R12A (SEQ | protein phosphatase | | | x | | | | |
| | | ID NO: 93) | 1, regulatory | | | | | | | |
| | | | subunit 12A | | | | | | | |
| 94 | 843 | CASP10 (SEQ ID | caspase 10 | | | x | | | | |
| | | NO: 94) | | | | | | | | |
| 95 | 2308 | FOXO1 (SEQ ID | forkhead box O1 | | | x | | | | |
| | | NO: 95) | | | | | | | | |
| 96 | 29894 | CPSF1 (SEQ ID | cleavage and | x | | | | | | |
| | | NO: 96) | polyadenylation | | | | | | | |
| | | | specific factor 1, | | | | | | | |
| | | | 160 kDa | | | | | | | |
| 97 | 156 | GRK2 (SEQ ID | G protein-coupled | x | | | | | | |
| | | NO: 97) | receptor kinase 2 | | | | | | | |
| 98 | 10000 | AKT3 (SEQ ID | v-akt murine | x | | | | | | |
| | | NO: 98) | thymoma viral | | | | | | | |
| | | | oncogene homolog 3 | | | | | | | |
| 99 | 307 | ANXA4 (SEQ ID | annexin A4 | x | | | | | | |
| | | NO: 99) | | | | | | | | |
| 100 | 483 | ATP1B3 (SEQ ID | ATPase, Na+/K+ | x | | | | | | |
| | | NO: 100) | transporting, beta | | | | | | | |
| | | | 3 polypeptide | | | | | | | |
| 101 | 613 | BCR (SEQ ID | breakpoint cluster | x | | | | | | |
| | | NO: 101) | region | | | | | | | |
| 102 | 30850 | CDR2L (SEQ ID | cerebellar | x | | | | | | |
| | | NO: 102) | degeneration- | | | | | | | |
| | | | related protein 2- | | | | | | | |
| | | | like | | | | | | | |
| 103 | 4830 | NME1 (SEQ ID | NME/NM23 nucleoside | x | | | | | | |
| | | NO: 103) | diphosphate kinase | | | | | | | |
| | | | 1 | | | | | | | |
| 104 | 10563 | CXCL13 (SEQ ID | chemokine (C-X-C | x | | | | | | |
| | | NO: 104) | motif) ligand 13 | | | | | | | |
| 105 | 6374 | CXCL5 (SEQ ID | chemokine (C-X-C | x | | | | | | |
| | | NO: 105) | motif) ligand 5 | | | | | | | |
| 106 | 22826 | DNAJC8 (SEQ ID | DnaJ (Hsp40) | x | | | | | | |
| | | NO: 106) | homolog, subfamily | | | | | | | |
| | | | C, member 8 | | | | | | | |
| 107 | 1845 | DUSP3 (SEQ ID | dual specificity | x | | | | | | |
| | | NO: 107) | phosphatase 3 | | | | | | | |
| 108 | 1938 | EEF2 (SEQ ID | eukaryotic | x | | | | | | |
| | | NO: 108) | translation | | | | | | | |
| | | | elongation factor 2 | | | | | | | |
| 109 | 9500 | MAGED1 (SEQ ID | melanoma antigen | x | | | | | | |
| | | NO: 109) | family D, 1 | | | | | | | |
| 110 | 9470 | EIF4E2 (SEQ ID | eukaryotic | | | | | x | | |
| | | NO: 110) | translation | | | | | | | |
| | | | initiation factor | | | | | | | |
| | | | 4E family member 2 | | | | | | | |
| 111 | 3329 | HSPD1 (SEQ ID | heat shock protein | | | | | x | | |
| | | NO: 111) | family D (Hsp60) | | | | | | | |
| | | | member 1 | | | | | | | |
| 112 | 3605 | IL17A (SEQ ID | interleukin 17A | | | | | x | | |
| | | NO: 112) | | | | | | | | |
| 113 | 4137 | MAPT (SEQ ID | microtubule- | | | | | x | | |
| | | NO: 113) | associated protein | | | | | | | |
| | | | tau | | | | | | | |
| 114 | 55703 | POLR3B (SEQ ID | polymerase (RNA) | | | | | x | | |
| | | NO: 114) | III (DNA directed) | | | | | | | |
| | | | polypeptide B | | | | | | | |
| 115 | 26037 | SIPA1L1 (SEQ | signal-induced | x | | | | x | | |
| | | ID NO: 115) | proliferation- | | | | | | | |
| | | | associated 1 like 1 | | | | | | | |
| 116 | 6613 | SUMO2 (SEQ ID | small ubiquitin- | | | | | x | | |
| | | NO: 116) | like modifier 2 | | | | | | | |
| 117 | 9325 | TRIP4 (SEQ ID | thyroid hormone | | | | | x | | |
| | | NO: 117) | receptor interactor | | | | | | | |
| | | | 4 | | | | | | | |
| 118 | 51271 | UBAP1 (SEQ ID | ubiquitin | x | | | | x | | |
| | | NO: 118) | associated protein | | | | | | | |
| | | | 1 | | | | | | | |
| 119 | 8945 | BTRC (SEQ ID | beta-transducin | x | | | | | | x |
| | | NO: 119) | repeat containing | | | | | | | |
| | | | E3 ubiquitin | | | | | | | |
| | | | protein ligase | | | | | | | |
| 120 | 1956 | EGFR (SEQ ID | epidermal growth | | | | | | | x |
| | | NO: 120) | factor receptor | | | | | | | |
| 121 | 2335 | FN1 (SEQ ID | fibronectin 1 | | | | | | | x |
| | | NO: 121) | | | | | | | | |
| 122 | 3855 | KRT7 (SEQ ID | keratin 7, type II | | | | | | | x |
| | | NO: 122) | | | | | | | | |
| 123 | 3913 | LAMB2 (SEQ ID | laminin, beta 2 | | | | | | | x |
| | | NO: 123) | (laminin S) | | | | | | | |
| 124 | 4286 | MITF (SEQ ID | microphthalmia- | | | | | | | x |
| | | NO: 124) | associated | | | | | | | |
| | | | transcription | | | | | | | |
| | | | factor | | | | | | | |
| 125 | 5493 | PPL (SEQ ID | periplakin | | | | | | | x |
| | | NO: 125) | | | | | | | | |
| 126 | 59307 | SIGIRR (SEQ ID | single | x | | | | | | x |
| | | NO: 126) | immunoglobulin and | | | | | | | |
| | | | toll-interleukin 1 | | | | | | | |
| | | | receptor (TIR) | | | | | | | |
| | | | domain | | | | | | | |
| 127 | 53340 | SPA17 (SEQ ID | sperm autoantigenic | | | | | | | x |
| | | NO: 127) | protein 17 | | | | | | | |
| 128 | 51684 | SUFU (SEQ ID | suppressor of fused | x | | | | | | x |
| | | NO: 128) | homolog | | | | | | | |
| | | | (Drosophila) | | | | | | | |
| 129 | 54472 | TOLLIP (SEQ ID | toll interacting | | | | | | | x |
| | | NO: 129) | protein | | | | | | | |
| 130 | 4796 | TONSL (SEQ ID | tonsoku-like, DNA | | | | | | | x |
| | | NO: 130) | repair protein | | | | | | | |
| 131 | 123 | PLIN2 (SEQ ID | perilipin 2 | x | | | | x | | |
| | | NO: 131) | | | | | | | | |
| 132 | 64326 | RFWD2 (SEQ ID | ring finger and WD | | | | | x | | |
| | | NO: 132) | repeat domain 2, E3 | | | | | | | |
| | | | ubiquitin protein | | | | | | | |
| | | | ligase | | | | | | | |
| 133 | 11194 | ABCB8 (SEQ ID | ATP-binding | x | | | | x | | |
| | | NO: 133) | cassette, sub- | | | | | | | |
| | | | family B (MDR/TAP), | | | | | | | |
| | | | member 8 | | | | | | | |
| 134 | 8878 | SQSTM1 (SEQ ID | sequestosome 1 | | | | | x | | |
| | | NO: 134) | | | | | | | | |
| 135 | 30848 | CTAG2 (SEQ ID | cancer/testis | x | | | | | | |
| | | NO: 135) | antigen 2 | | | | | | | |

The GeneID and Gene Symbol can be found on the NCBI website available at www.ncbi.hlm.nih.gov. More information about the gene can be found by accessing the NCBI website and entering the GeneID or Gene Symbol, for instance.

### Example 8: Identification of the Pre-Treatment Autoantibody Response in Melanoma Patients

The pre-treatment (T0 or baseline) autoantibody response of melanoma patients has the potential to predict clinical response or longer survival of melanoma patients. Serum samples from 193 melanoma patients were obtained before starting treatment with anti-CTLA-4 (ipilumumab), anti-PD-1 (nivolumab or pembrolizumab) or anti-CTLA-4/anti-PD-1 combination therapy. The autoantibody levels of serum samples from melanoma patients were compared with autoantibody profiles of 148 healthy volunteer samples using based statistical technique Significance of microarrays (SAM).

The preexisting autoantibody repertoire of melanoma patients at baseline is shown in Table 2. Autoantibody targets in table 2 are top-down ranked by their calculated SAM Score d. The correlation of baseline autoantibodies with different clinical endpoints such as the occurrence of irAEs or clinical response (disease control rate, DCR) is shown in separate tables (T).

Table 2 shows 36 autoantibody targets with higher reactivity in the melanoma group compared healthy controls, which is indicated by a positive fold change: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, and NOVA2.

There were also 11 autoantibodies with lower reactivity (negative fold-change) in the melanoma group compared to healthy control samples: SNRPA, NRIP1, UBAP1, TEX264, PLIN2, LAMC1, CENPH, USB1, ABCB8, C15orf48/NMES1, and MAGED1.

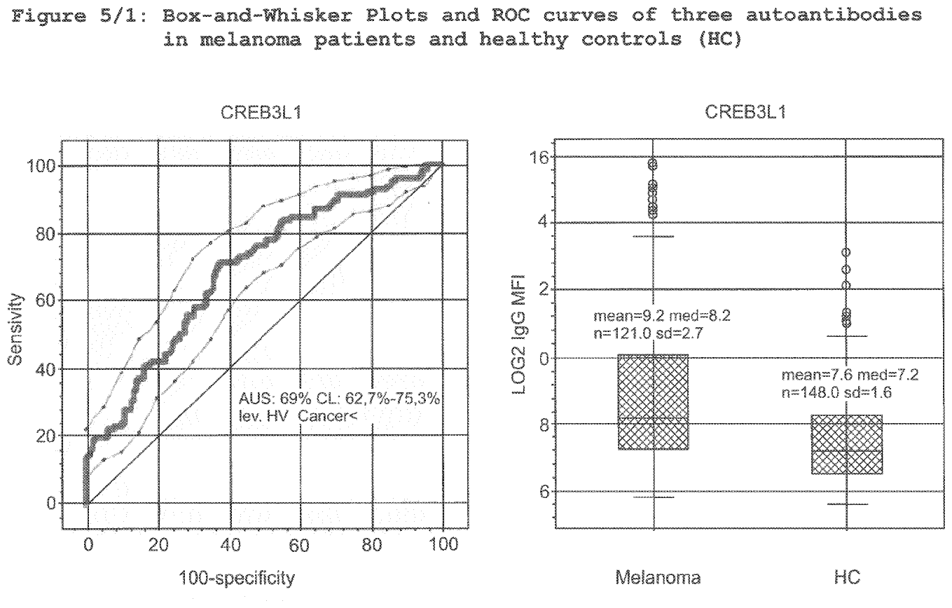

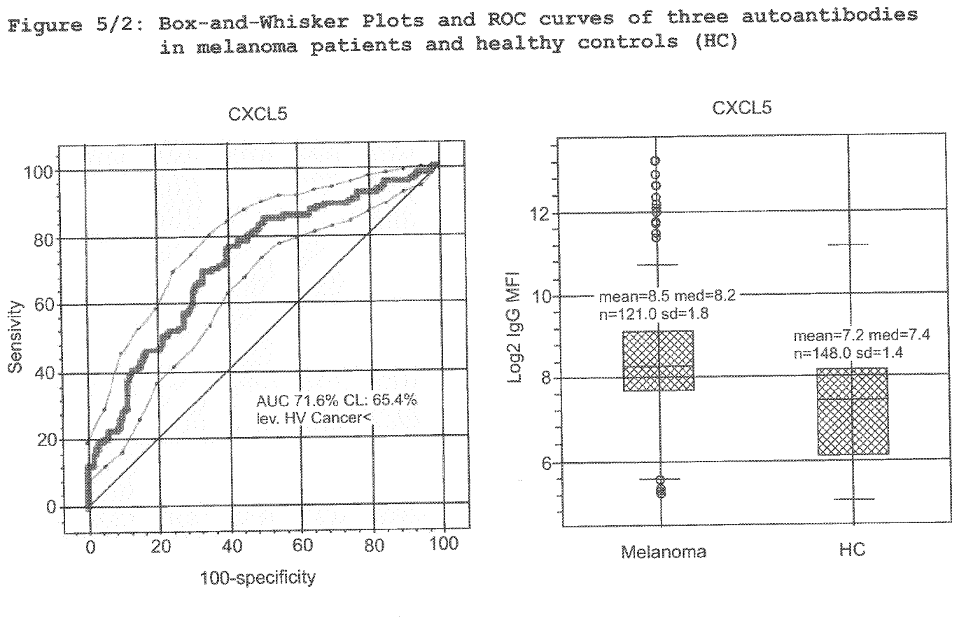

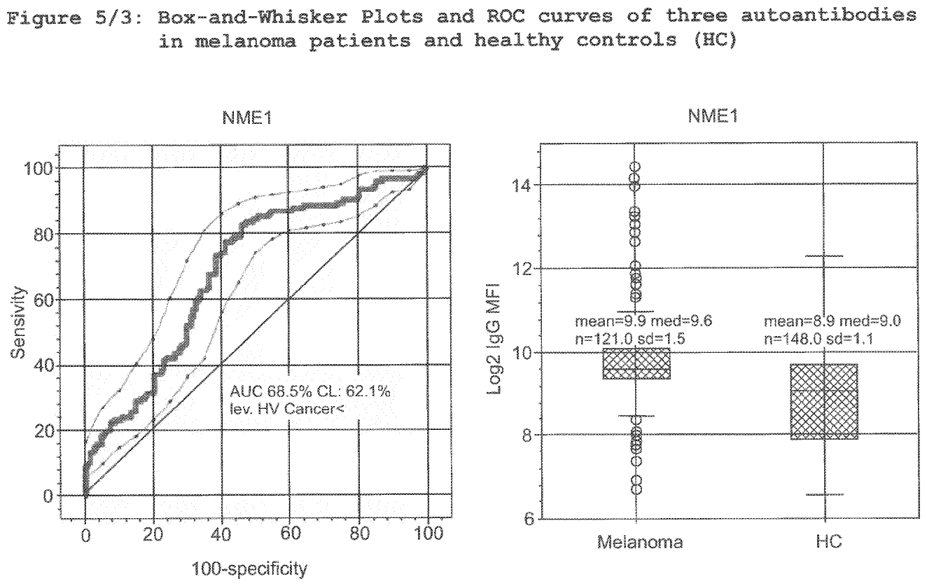

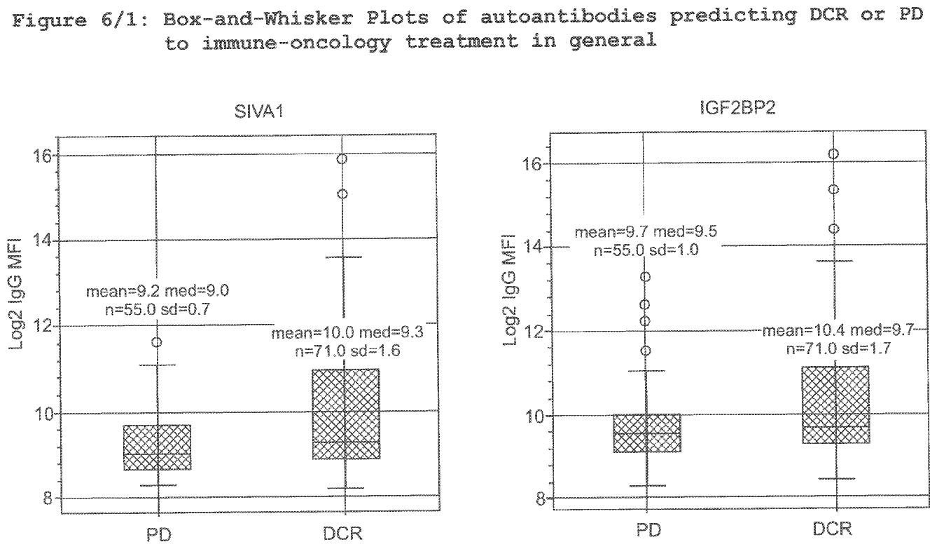

FIG. 5 shows Box-and-Whisker plots and ROC curves of three autoantibodies, CREB3L1, CXCL5, and NME1, with higher reactivity in serum samples of melanoma patients compared to healthy controls. The calculated area under the curve (AUC) of CREB3L1, CXCL5, and NME1 is 69%, 72%, and 69%, respectively.

CREB3L1 is also referred to as “Cyclic AMP-responsive element-binding protein 3-like protein 1”, “Old astrocyte specifically-induced substance”, and OASIS. CREB3L1 is a transcription factor that represses expression of genes regulating metastasis, invasion, and angiogenesis. Baseline anti-CREB3L1 antibodies also predict the development of irAE following treatment with different checkpoint inhibitors (Table 4) including ipilimumab (Table 6).

CXCL5 is also referred to as “C-X-C motif chemokine 5”, “Epithelial-derived neutrophil-activating protein 78”, “Neutrophil-activating peptide ENA-78”, “Small-inducible cytokine B5”, and ENA78. CXCL5 is a chemokine, which stimulates the chemotaxis of neutrophils possessing angiogenic properties following binding the binds to cell surface chemokine receptor CXCR2. Tumor-associated neutrophils are increasingly recognized for their ability to promote tumor progression, mediate resistance to therapy, and regulate immunosuppression via the CXCL5/CXCR2 axis.

NME1 is also referred to as “Nucleoside diphosphate kinase A (EC:2.7.4.6)”, “NDP kinase A”, “Granzyme A-activated DNase”, “Metastasis inhibition factor nm23”, “Tumor metastatic process-associated protein”, GAAD, NM23-H1, NME1, NDPKA, and. NM23. Expression of the metastasis suppressor NME1 in melanoma is associated with reduced cellular motility and invasion in vitro and metastasis.

The three examples demonstrate that the autoantibody response of tumor patients is directed against a diverse set of proteins, which play a role in cancer processes.

TABLE 2
Autoantibody profile of melanoma patients

| | | | SAM | SAM | | | | | | |
|---|---|---|---|---|---|---|---|---|---|---|
| | | | Score d HC | fold-change | | | | | | |
| Marker No | Gene ID | Gene Symbol | vs Melanoma | HC vs Melanoma | T3 | T4 | T5 | T6 | T7 | T8 |
| 61 | 6181 | RPLP2 | 6.59 | 3.08 | x | | | | | |
| 11 | 1485 | CTAG1B | 5.70 | 3.70 | x | x | | | | |
| 108 | 1938 | EEF2 | 5.40 | 2.51 | x | | | | | |
| 105 | 6374 | CXCL5 | 5.40 | 2.45 | x | | | | | |
| 106 | 22826 | DNAJC8 | 5.04 | 2.26 | x | | | | | |
| 3 | 90993 | CREB3L1 | 4.94 | 2.90 | x | | x | | x | |
| 98 | 10000 | AKT3 | 4.86 | 1.77 | x | | | | | |
| 104 | 10563 | CXCL13 | 4.86 | 1.88 | x | | | | | |
| 103 | 4830 | NME1 | 4.73 | 1.98 | x | | | | | |
| 99 | 307 | ANXA4 | 4.53 | 1.73 | x | | | | | |
| 30 | 11214 | AKAP13 | 4.50 | 1.96 | x | | | | | |
| 102 | 30850 | CDR2L | 4.48 | 2.19 | x | | | | | |
| 100 | 483 | ATP1B3 | 4.46 | 1.71 | x | | | | | |
| 107 | 1845 | DUSP3 | 4.38 | 1.92 | x | | | | | |
| 62 | 6382 | SDC1 | 4.10 | 1.47 | x | | | | | |
| 96 | 29894 | CPSF1 | 4.09 | 1.83 | x | | | | | |
| 97 | 156 | GRK2 | 4.04 | 2.17 | x | | | | | |
| 63 | 6434 | TRA2B | 4.04 | 1.36 | x | | | | | |
| 101 | 613 | BCR | 4.01 | 1.57 | x | | | | | |
| 13 | 1457 | CSNK2A1 | 4.01 | 1.87 | x | | | | | x |
| 74 | 408 | ARRB1 | 3.91 | 1.80 | x | | | | | x |
| 88 | 2870 | GRK6 | 3.68 | 1.43 | x | x | | | | |
| 135 | 30848 | CTAG2 | 3.54 | 2.04 | x | | | | | |
| 10 | 4282 | MIF | 2.08 | 1.26 | x | x | | | | |
| 27 | 2065 | ERBB3 | 1.95 | 1.23 | x | | x | | | |
| 128 | 51684 | SUFU | 1.92 | 1.27 | x | | | | | |
| 119 | 8945 | BTRC | 1.90 | 1.33 | x | | | | | |
| 126 | 59307 | SIGIRR | 1.87 | 1.39 | x | | | | | |
| 115 | 26037 | SIPA1L1 | 1.83 | 1.34 | x | | | | x | |
| 72 | 60 | ACTB | 1.75 | 1.31 | x | | | | | x |
| 73 | 4302 | MLLT6 | 1.75 | 1.31 | x | | | | | x |
| 86 | 6464 | SHC1 | 1.70 | 1.20 | x | | | | | x |
| 12 | 10486 | CAP2 | 1.64 | 1.23 | x | | x | | | |
| 15 | 10243 | GPHN | 1.63 | 1.19 | x | x | | x | | |
| 60 | 361 | AQP4 | 1.62 | 1.24 | x | x | | | | |
| 8 | 4858 | NOVA2 | 1.52 | 1.42 | x | | | | | x |
| 56 | 6626 | SNRPA | −1.66 | 0.75 | x | | | | | x |
| 50 | 8204 | NRIP1 | −1.89 | 0.72 | x | | | | | x |
| 118 | 51271 | UBAP1 | −1.90 | 0.72 | x | | | | x | |
| 2 | 51368 | TEX264 | −2.19 | 0.65 | x | | x | | | x |
| 131 | 123 | PLIN2 | −2.20 | 0.65 | x | | | | x | |
| 78 | 3915 | LAMC1 | −2.25 | 0.64 | x | | | | | x |
| 40 | 64946 | CENPH | −2.25 | 0.70 | x | | | x | | |
| 37 | 79650 | USB1 | −2.56 | 0.73 | x | | | x | | |
| 133 | 11194 | ABCB8 | −2.61 | 0.77 | x | | | | x | |
| 9 | 84419 | C15orf48; NMES1 | −2.66 | 0.74 | x | x | | | | x |
| 109 | 9500 | MAGED1 | −4.28 | 0.65 | x | | | | | |

### Example 9: Identification of Autoantibodies Associated or Predicting Survival and Clinical Response to Immune-Oncology Agents

The role of B cells and their secreted products in driving anti-cancer immunity is only insufficiently understood. Autoantibodies produced by B cells may have both pro- and anti-tumor effects. Thus, autoantibodies may serve as biomarkers of the general immune fitness of a cancer patients and his ability to respond to immune-oncology agents.

The autoantibody reactivity of serum samples from 193 melanoma patients treated with anti-CTLA-4 (ipilumumab), anti-PD-1 (nivolumab or pembrolizumab) or anti-CTLA-4/anti-PD-1 combination therapy was analyzed. To evaluate the difference in autoantibody levels between the clinical outcomes DCR and PD, the statistical test SAM was applied. Spearman's rank correlation analysis was used to evaluate the association between autoantibody levels and overall survival (OS).

Ten autoantibodies predicted a clinical response referred to as “disease control rate” (DCR) to immune-oncology treatments in general.

Six baseline autoantibodies directed towards SIVA1, IGF2BP2, AQP4, C15orf48, GPHN, and CTAG1B appear to be predictors of DCR.

Four baseline autoantibodies directed towards GRK6, FGFR1, MIF, and GRAMD4 appear to be predictors of non-response or progressive disease (PD) to immune-oncology treatment in general.

Anti-PAPOLG antibodies were weakly associated with overall survival (Spearman's rank correlation coefficient r=0.32).

Table 3 shows autoantibodies associated with OS and DCR in melanoma patients treated with different checkpoint inhibitors.

TABLE 3
Autoantibodies associated OS and DCR in melanoma patients

| | | | | SAM | SAM |
|---|---|---|---|---|---|
| | Gene | Gene | Spearman's | Score d | fold-change |
| ID | ID | Symbol | R-value OS | DCR at T0 | DCR at T0 |
| 59 | 10572 | SIVA1 | 0.02 | 2.12 | 1.66 |
| 14 | 10644 | IGF2BP2 | 0.07 | 1.99 | 1.65 |
| 60 | 361 | AQP4 | −0.15 | 1.85 | 1.49 |
| 9 | 84419 | C15orf48 | 0.11 | 1.85 | 1.46 |
| 15 | 10243 | GPHN | 0.09 | 1.84 | 1.40 |
| 11 | 1485 | CTAG1B | 0.08 | 1.83 | 2.21 |
| 21 | 64895 | PAPOLG | 0.32 | 0.24 | 1.05 |
| 88 | 2870 | GRK6 | −0.12 | −1.80 | 0.71 |
| 90 | 2260 | FGFR1 | −0.07 | −1.86 | 0.67 |
| 10 | 4282 | MIF | −0.07 | −1.88 | 0.67 |
| 1 | 23151 | GRAMD4 | −0.18 | −1.92 | 0.68 |

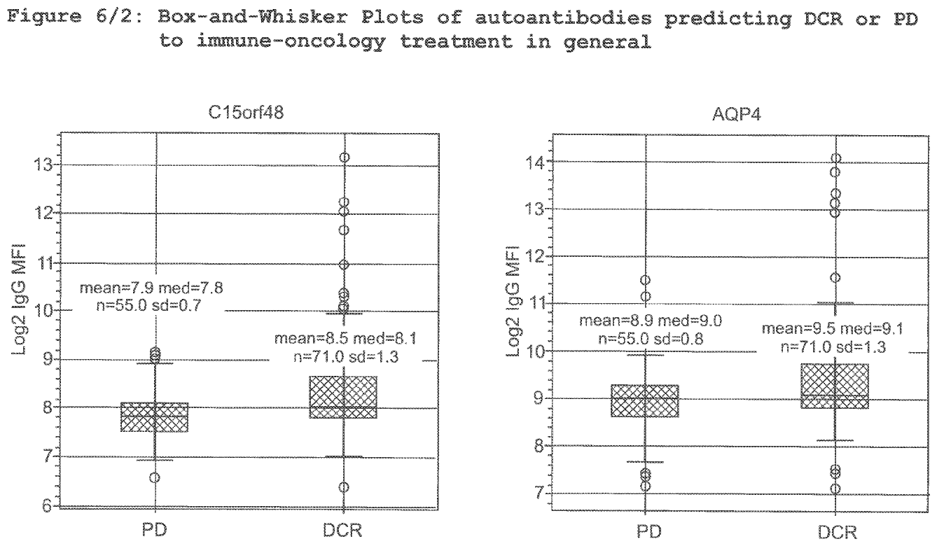

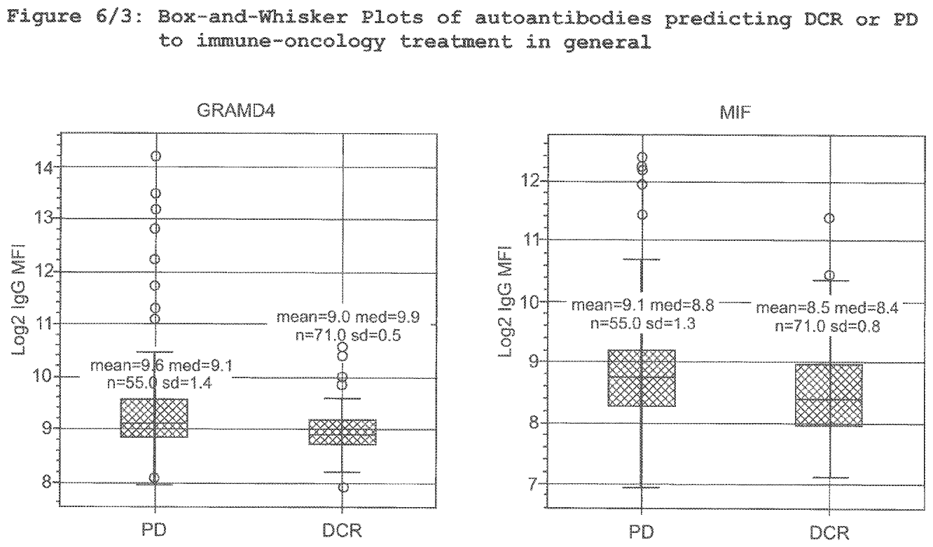

FIG. 6 shows four baseline autoantibodies, SIVA1, IGF2BP2, AQP4, and C15orf48, which predict DCR and two baseline autoantibodies, MIF and GRAMD4, which predict PD to checkpoint inhibitor treatment in general.

SIVA1 is also referred to as “Apoptosis regulatory protein Siva”, “CD27-binding protein”, CD27BP, or SIVA1. SIVA1 plays an important role in the apoptotic (programmed cell death) pathway induced by the CD27 antigen, a member of the tumor necrosis factor receptor (TFNR) superfamily.

IGF2BP2 is also referred to as “Insulin-like growth factor 2 mRNA-binding protein 2”, “Hepatocellular carcinoma autoantigen p62”, “IGF-II mRNA-binding protein 2”, “VICKZ family member 2”, IGF2BP2, IMP2, or VICKZ2. The gene encoding IGF2BP2 is amplified and overexpressed in many human cancers, accompanied by a poorer prognosis (Dai et al., 2017). Higher baseline anti-IGF2BP2 antibodies were also found in melanoma patients who achieve DCR following treatment with the PD-1/PD-L1 pathway blocker pembrolizumab.

AQP4 is also referred to as “Aquaporin-4”, “Mercurial-insensitive water channel”, MIWC, or WCH4. AQP4 is a water channel protein, predominantly found in tissues of neuronal origin. Anti-AQP4 antibodies are found in the autoimmune disorder, neuromyelitis optica, NMO, which affects the optics nerves and spinal cord of individuals. Higher levels of anti-AQP4 antibodies were found in melanoma patients compared to healthy controls.

C15orf48 is also referred to as “normal mucosa of esophagus-specific gene 1 protein”, Protein FOAP-11, MIR147BHG, or NMES1. Higher baseline anti-IGF2BP2 antibodies were also found were found in melanoma patients compared to healthy controls and predict clinical response as defined as DCR following treatment with the PD-1/PD-L1 pathway blocker pembrolizumab.

GRAMD4 is also referred to as “GRAM domain-containing protein 4”, “Death-inducing protein”, DIP, or KIAA0767. GRAMD4 has been reported as a pro-apoptotic protein. Higher baseline levels of anti-GRAMD4 antibodies were also associated with PD and shorter overall survival in melanoma patients treated with the CTLA-4 inhibitor ipilimumab.

MIF is also referred to as “Macrophage migration inhibitory factor (EC:5.3.2.1)”, “Glycosylation-inhibiting factor”, “L-dopachrome tautomerase (EC:5.3.3.12)”, or GIF. MIF is a proinflammatory cytokine, which is overexpressed in malignant melanoma. Higher baseline levels of anti-MIF antibodies were found in melanoma patients compared to healthy controls and in melanoma patients who do not develop irAEs after treatment with the PD-1/PD-L1 pathway blocker pembrolizumab.

### Example 10: Identification of Baseline Autoantibodies Predicting irAE in Melanoma Patients Following Treatment with Different Checkpoint Inhibitors

Despite important clinical benefits, checkpoint inhibitors area associated with immune-related adverse events (irAEs) The mechanisms by which checkpoint inhibitors induce irAEs are not completely understood. It is believed that by blocking negative checkpoints a general immunologic enhancement occurs. It is also possible that by unleashing the immune-checkpoints that control tolerance, autoreactive lymphocytes are activated, which could be either T cells or B cells. It is well known that in autoimmune diseases autoreactive B cells produce autoantibodies that can induce tissue damage via ADCC. Thus, epitope spreading towards self-antigens may be an indicator for irAEs.

Autoantibodies predicting irAEs were identified in pre-treatment samples from patients receiving different checkpoint inhibitors such as anti-CTLA-4, anti-PD-1 or combination therapies of anti-CTLA-4 and anti-PD-1. To evaluate the difference in autoantibody levels between patients experiencing an irAE and those who do not, the statistical test SAM was applied.

Table 4 includes 12 autoantibodies reacting with TEX264, CREB3L1, HSPA1B, SPTB, MUC12, ERBB3, ATG4D, CASP10, FOXO1, FRS2, and PPP1R12A, which appear to predict irAEs in baseline samples. A positive fold-change indicates higher autoantibody levels in the group of melanoma patients who experience an irAE, whereas a negative fold-change indicates higher autoantibody levels in patients who do not develop an irAE. Table 4 includes five autoantibodies, HSPA2, SMAD9, HIST2H2AA3, S100A8, and SDCBP, which predict that patients having higher autoantibody levels do not develop an irAE.

TABLE 4
Baseline autoantibodies predicting irAE in melanoma patients
following treatment with different checkpoint inhibitors

| | | | SAM | SAM |
|---|---|---|---|---|
| | Gene | Gene | Score.d.irAE | Fold.Change |
| ID | ID | Symbol | at T0 | irAE at T0 |
| 2 | 51368 | TEX264 | 2.41 | 1.93 |
| 3 | 90993 | CREB3L1 | 2.33 | 2.42 |
| 17 | 3304 | HSPA1B | 2.17 | 1.63 |
| 18 | 6710 | SPTB | 2.17 | 1.63 |
| 57 | 10071 | MUC12 | 2.06 | 1.49 |
| 27 | 2065 | ERBB3 | 2.04 | 1.36 |
| 28 | 84971 | ATG4D | 2.03 | 1.36 |
| 94 | 843 | CASP10 | 2.02 | 1.36 |
| 95 | 2308 | FOXO1 | 1.99 | 1.83 |
| 5 | 10818 | FRS2 | 1.92 | 1.72 |
| 93 | 4659 | PPP1R12A | 1.90 | 1.59 |
| 12 | 10486 | CAP2 | 1.87 | 1.43 |
| 31 | 3306 | HSPA2 | −1.85 | 0.72 |
| 32 | 4093 | SMAD9 | −1.96 | 0.71 |
| 58 | 8337 | HIST2H2AA3 | −2.04 | 0.73 |
| 6 | 6279 | S100A8 | −2.15 | 0.76 |
| 16 | 6386 | SDCBP | −2.73 | 0.60 |

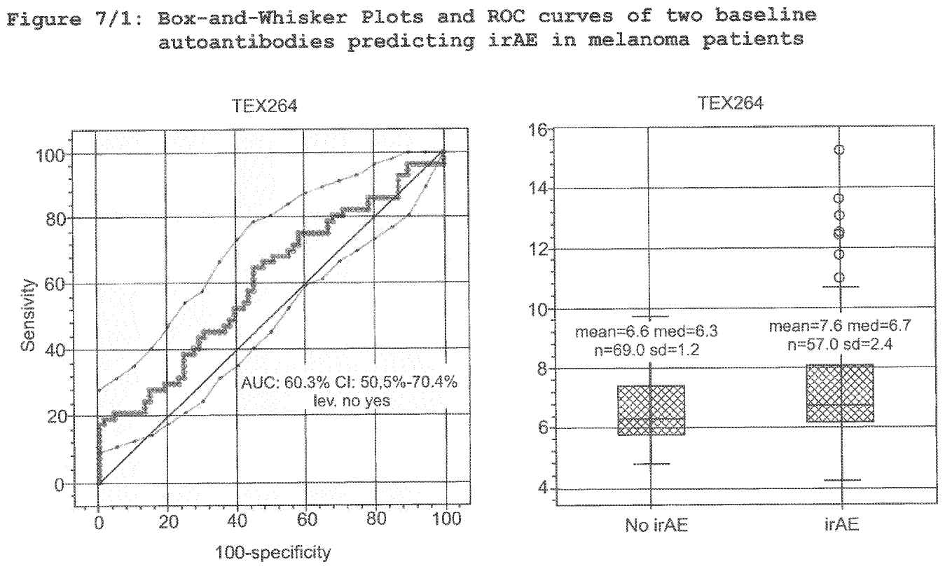

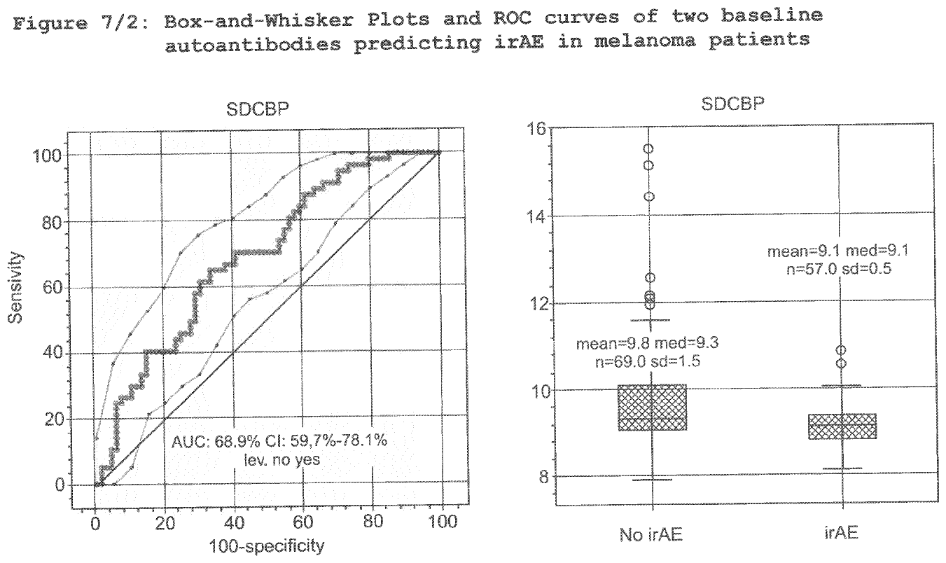

FIG. 7 shows Box-and-Whisker Plots and ROC curves of baseline levels of anti-TEX264 and anti-SDCBP antibodies that allow to discriminate patients developing irAE from those who do not develop irAE in response to checkpoint inhibitor treatment. The calculated area under the curve (AUC) of anti-TEX264 and anti-SDCBP is 60% and 69%, respectively.

TEX264 is also referred to as “Testis-expressed protein 264”, or “Putative secreted protein Zsig11”. The function of the gene encoding TEX264 is currently unknown. Elevated baseline anti-TEX264 antibodies also predict clinical response as defined as DCR and the development of irAEs in patients treated with the anti-PD-1 blocker pembrolizumab.

SDCBP is also referred to as “syntenin-1”, “Melanoma differentiation-associated protein 9”, MDA-9, “Pro-TGF-alpha cytoplasmic domain-interacting protein 18”, TACIP18, “Scaffold protein Pbp1”, “Syndecan-binding protein 1”, MDA9, or SYCL. SDCBP is expressed in melanoma and influences metastasis by regulating both tumor cells and the microenvironment (Das et al., 2012). Higher baseline anti-SDCBP antibodies were also found in patients who do not develop irAEs following treatment with anti-CTLA-4 inhibitor ipilimumab.

### Example 11: Identification of Autoantibodies Associated or Predicting Survival and Clinical Response to Ipilimumab Treatment

One of the reasons to terminate a patient's cancer therapy or to change the therapy is disease progression.

To identify autoantibodies that allow to identify patients who benefit from ipilimumab therapy, serum samples from 82 melanoma patients treated with ipilimumab were analyzed.

Biomarkers correlating with progression-free survival (PFS) or overall survival (OS) were calculated using Spearman's correlation. To evaluate the difference in autoantibody levels between the clinical outcomes DCR and PD, the statistical test SAM was applied.

Table 5 shows 13 autoantibodies, FRS2, GPHN, BIRC5, EIF3E, CENPH, PAPOLG, HUS1, GNG12, CCDC51, USB1, GRAMD4, RPS6KA1, and BCL7B, correlating positively or negatively with PFS, OS, or predict DCR or PD in baseline samples.

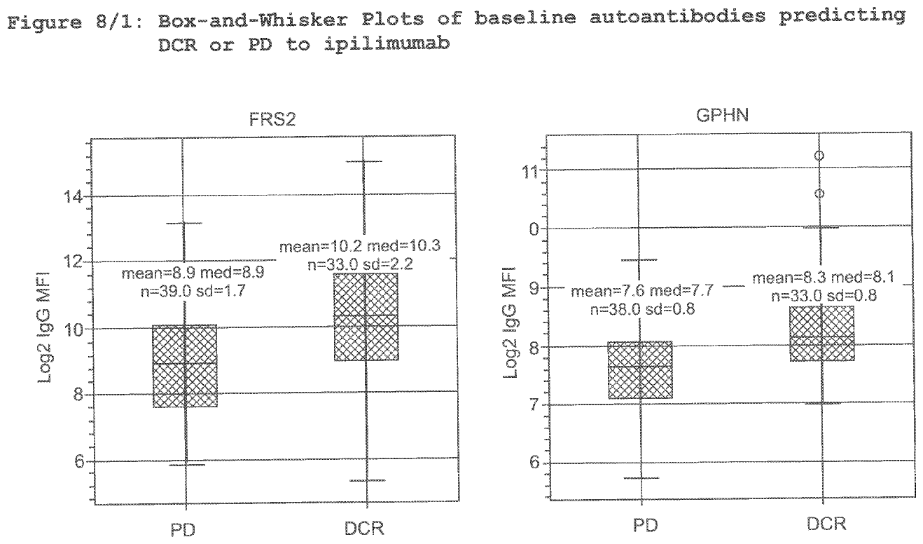

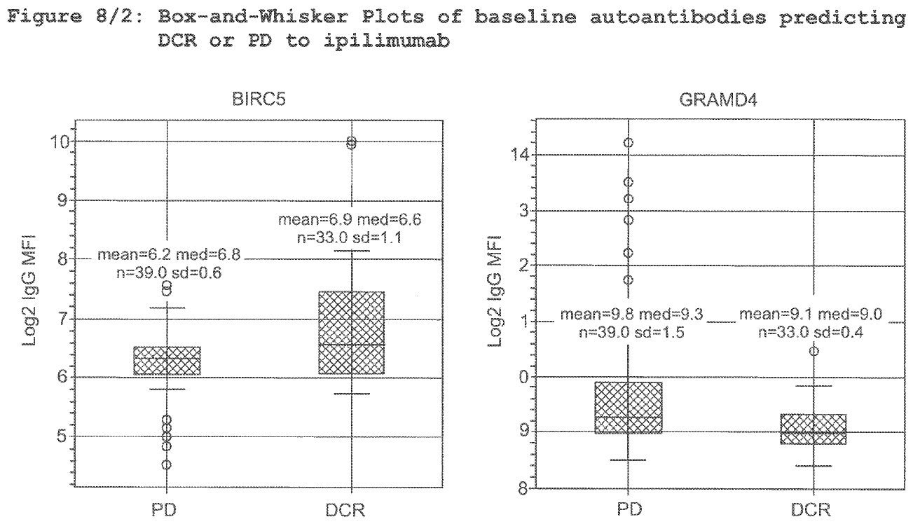

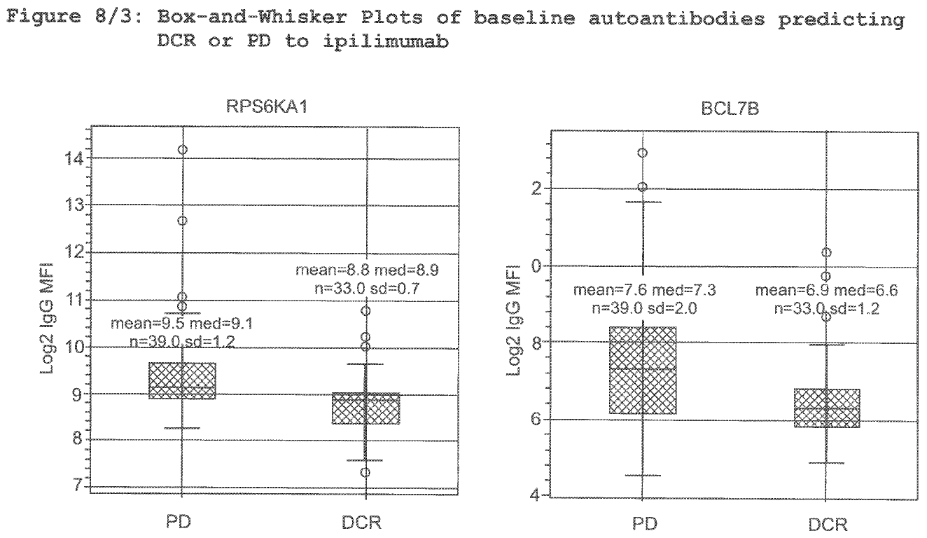

FIG. 8 shows Box-and-Whisker plots of six baseline autoantibodies, FRS2, GPHN, BIRC5, GRAMD4, RPS6Ka2, and BCL7B, predicting DCR or PD to ipilimumab.

BIRC5 is also known as “Baculoviral IAP repeat-containing protein 5”, “Apoptosis inhibitor 4”, “Apoptosis inhibitor surviving”API4, or IAP4. BIRC5 is overexpressed in human cancer and plays a role in inhibition of apoptosis, resistance to chemotherapy and aggressiveness of tumors (Garg et al., 2016).

FRS2 is also known as “Fibroblast growth factor receptor substrate 2”, “FGFR-signaling adaptor SNT”, “Suc1-associated neurotrophic factor target 1”, or SNT-1. FRS2 is overexpressed and amplified in several cancer types. It serves as a docking protein for receptor tyrosine kinases, which mediate proliferation, survival, migration, and differentiation (Luo and Hahn, 2015). Baseline levels of anti-FRS2 also predict both response to anti-CTLA-4 treatment (Table 5) and the development of irAE (Table 6).

BCL7B also known as § B-cell CLL/lymphoma 7 protein family member B″ is a member of the BCL7 gene family, which is involved in the modulation of multiple pathways, including Wnt and apoptosis. The BCL7 family is involved in cancer incidence, progression, and development (Uehara et al., 2015).

RPS6KA1 is also known as “Ribosomal protein S6 kinase alpha-1 (EC:2.7.11.1)”, “MAP kinase-activated protein kinase 1a”, p90RSK1, RSK-1, or MAPKAPK1A. The RSK (90 kDa ribosomal S6 kinase) family comprises a group of highly related serine/threonine kinases that regulate diverse cellular processes, including cell growth, proliferation, survival and motility. Dysregulated RSK expression and activity has been associated with multiple cancer types (Houles and Roux, 2017).

GPHN is also known as “Gephyrin”, “Molybdopterin adenylyltransferase (EC:2.7.7.75)”, MPT, or KIAA1385. Gephyrin is a 93 kDa multi-functional protein that is a component of the postsynaptic protein network of inhibitory synapses. In non-neuronal tissues, the encoded protein is also required for molybdenum cofactor biosynthesis, a cofactor of sulfite oxidase, aldehyde oxidase, and xanthine oxidoreductase (Smolinsky et al., 2008). Besides predicting response to anti-CTLA-4 therapy, GPHN is also a useful marker to discriminate melanoma patients from normal humans (Table 2) and predicts DCR in melanoma patients treated with different checkpoint inhibitors (Table 3).

TABLE 5
Autoantibodies associated with PFS, OS and DCR
in melanoma patients treated with ipilimumab

| | | | | | SAM | SAMR |
|---|---|---|---|---|---|---|
| | | | R-value | R-value | Score.d.DCR | Fold.Change |
| ID | Gene ID | Gene Symbol | PFS | OS | at T0 | DCR at T0 |
| 5 | 10818 | FRS2 | 0.21 | 0.2 | 2.23 | 2.55 |
| 15 | 10243 | GPHN | 0.16 | 0.24 | 2.18 | 1.68 |
| 33 | 332 | BIRC5 | 0.05 | 0.06 | 1.8 | 1.54 |
| 39 | 3646 | EIF3E | 0.08 | 0.33 | 1.09 | 1.31 |
| 40 | 64946 | CENPH | 0.18 | 0.31 | 0.88 | 1.31 |
| 21 | 64895 | PAPOLG | 0.28 | 0.37 | 0.53 | 1.14 |
| 43 | 3364 | HUS1 | 0.34 | 0.16 | −0.01 | 1 |
| 41 | 55970 | GNG12 | 0.32 | 0.24 | −0.21 | 0.95 |
| 42 | 79714 | CCDC51 | −0.32 | −0.15 | −0.34 | 0.88 |
| 37 | 79650 | USB1 | −0.16 | −0.06 | −1.81 | 0.6 |
| 1 | 23151 | GRAMD4 | −0.3 | −0.32 | −1.92 | 0.58 |
| 36 | 6195 | RPS6KA1 | −0.2 | −0.17 | −1.92 | 0.61 |
| 38 | 9275 | BCL7B | −0.1 | −0.17 | −1.95 | 0.48 |

### Example 12: Identification of Autoantibodies Associated with irAEs in Patients Treated with Ipilimumab

Autoantibodies predicting irAEs were identified in pre-treatment samples from patients receiving anti-CTLA-4 therapy. To evaluate the difference in autoantibody levels between patients experiencing an irAE and those who do not, the statistical test SAM was applied.

Table 6 includes 13 autoantibodies reacting with EOMES, CREB3L1, FRS2, PLIN2, SIPA1L1, ABCB8, MAPT, ATG4D, XRCC5, XRCC6, UBAP1, TRIP4, and EIF4E2, which appear to predict irAEs in baseline samples. A positive fold-change indicates higher autoantibody levels in the group of melanoma patients who experience an irAE, whereas a negative fold-change indicates higher autoantibody levels in patients who do not develop an irAE.

Table 4 includes eight autoantibodies, POLR3B, ELMO2, SUMO2, RFWD2, SQSTM1, SDCBP, HSPD1, and IL17A, which predict that patients having higher autoantibody levels do not develop an irAE.

TABLE 6
Baseline autoantibodies predicting irAEs in
melanoma patients treated with ipilimumab

| | | | SAM | SAM |
|---|---|---|---|---|
| | Gene | Gene | Score.d.irAE | Fold.Change |
| ID | ID | Symbol | at T0 | irAE at T0 |
| 26 | 8320 | EOMES | 2.30 | 2.82 |
| 3 | 90993 | CREB3L1 | 2.28 | 2.60 |
| 5 | 10818 | FRS2 | 2.11 | 2.28 |
| 131 | 123 | PLIN2 | 2.11 | 2.13 |
| 115 | 26037 | SIPA1L1 | 1.96 | 1.85 |
| 133 | 11194 | ABCB8 | 1.93 | 1.47 |
| 113 | 4137 | MAPT | 1.87 | 1.58 |
| 28 | 84971 | ATG4D | 1.83 | 1.34 |
| 24 | 7520 | XRCC5 | 1.82 | 1.55 |
| 25 | 2547 | XRCC6 | 1.82 | 1.55 |
| 118 | 51271 | UBAP1 | 1.81 | 1.75 |
| 117 | 9325 | TRIP4 | 1.81 | 1.57 |
| 110 | 9470 | EIF4E2 | 1.81 | 1.62 |
| 114 | 55703 | POLR3B | −1.84 | 0.55 |
| 29 | 63916 | ELMO2 | −1.89 | 0.55 |
| 116 | 6613 | SUMO2 | −1.91 | 0.63 |
| 132 | 64326 | RFWD2 | −1.99 | 0.67 |
| 134 | 8878 | SQSTM1 | −2.02 | 0.69 |
| 16 | 6386 | SDCBP | −2.03 | 0.64 |
| 111 | 3329 | HSPD1 | −2.10 | 0.44 |
| 112 | 3605 | IL17A | −2.19 | 0.60 |

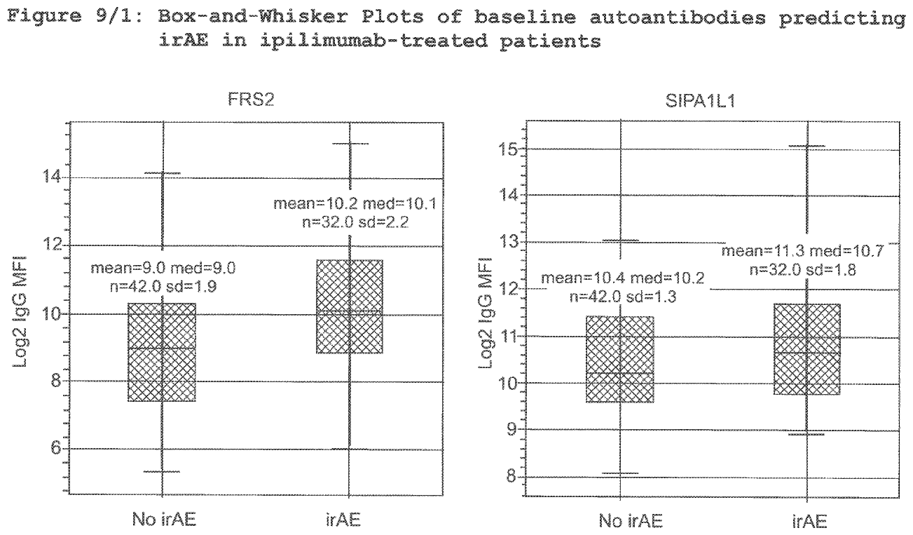

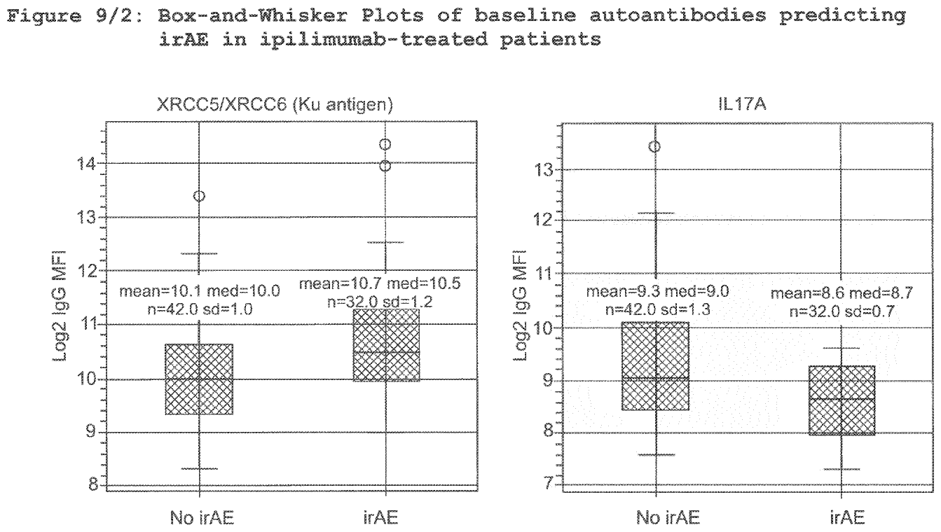

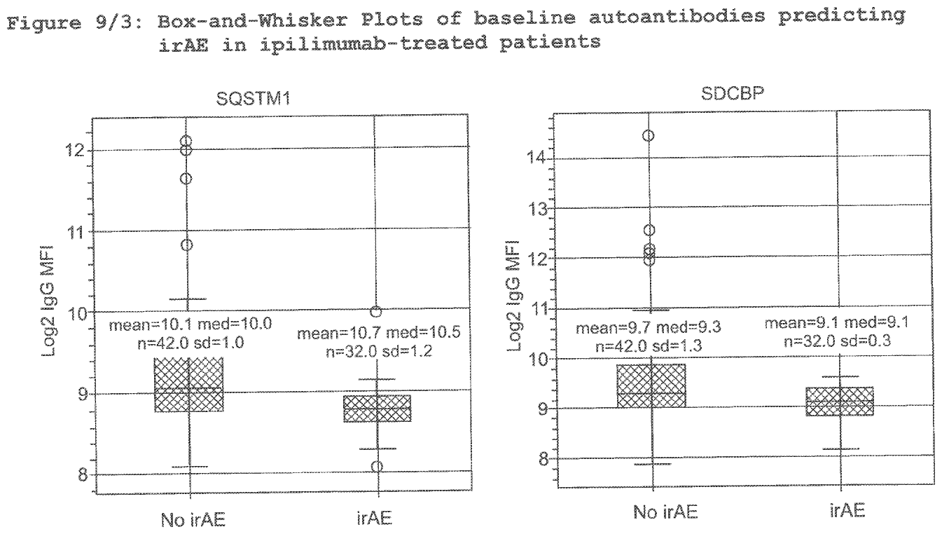

FIG. 9 shows Box-and-Whisker plots of six baseline autoantibodies, FRS2, SIPA1L1, XRCC5/XRCC6, IL17A, SQSTM1, and SDCBP, which are associated with the development of irAE in ipilimumab-treated patients.

Baseline levels of FRS2 predict both response to ipilimumab (Table 5) and the development of irAEs (Table 6).

SIPA1L1 is also known as “signal-induced proliferation-associated 1-like protein 1”, “High-risk human papilloma viruses E6 oncoproteins targeted protein 1”, E6TP1, or. KIAA0440. Besides predicting the development of irAEs, SIPA1L1 is also a useful marker to discriminate melanoma patients from normal humans (Table 2).

A dimer of the antigens XRCC5 and XRCC6 form the Lupus Ku autoantigen protein. Higher baseline levels of autoantibodies to XRCC5/XRCC6 predict the development of irAE in ipilimumab treated patients. XRCC5 is also known as “X-ray repair cross-complementing protein 5”, Lupus Ku autoantigen protein p86, Ku80, or Ku86. XRCC6 is also known as “X-ray repair cross-complementing protein 6”, 70 kDa subunit of Ku antigen, Lupus Ku autoantigen protein p70, Ku70, or thyroid-lupus autoantigen. Besides predicting the development of iRAE following anti-CTLA-4 therapy, XRCC5/XRCC6 also predict clinical response defined as DCR in melanoma patients treated with the PD-1/PD-L1 pathway blocker pembrolizumab (Table 7).

Higher levels of anti-IL17A antibodies are found in patients who do not develop irAEs following ipilimumab treatment. IL17A is also known as “interleukin 17A”, CTLA8; or IL-17. IL17 and is a proinflammatory cytokine produced by activated T cells.

SQSTM1 is also known as “sequestosome 1”, p60, p62, A170, DMRV, OSIL, PDB3, ZIPS, p62B, NADGP, or FTDALS3. SQSTM1 is an autophagosome cargo protein that targets other proteins that bind to it for selective autophagy. It is also interacts with signaling molecules to promote the expression of inflammatory genes (Moscat et al., 2016). Anti-SQSTM1 antibodies are found in melanoma patients who do not develop irAE following ipilimumab treatment.

### Example 13: Identification of Autoantibodies Associated or Predicting Survival and Clinical Response to Pembrolizumab Treatment

To identify autoantibodies that allow to identify patients who benefit from treatment with PD-1/PD-L1 pathway inhibitors, serum samples from 41 melanoma patients treated with pembrolizumab were analyzed.

Biomarkers correlating with progression-free survival (PFS) or overall survival (OS) were calculated using Spearman's correlation. To evaluate the difference in autoantibody levels between the clinical outcomes DCR and PD, the statistical test SAM was applied.

Table 7 lists 42 autoantibody targets, which are associated with response or non-response to pembrolizumab therapy: NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, FGA, CALR, GNAI2, IL36RN, S100A14, MMP3, SHC1, CSNK2A1, DFFA, LAMC1, S100A8, HDAC1, MSH2, CEACAM5, DHFR, and ARRB1.

Higher serum levels of ten autoantibodies were positively correlated with longer overall survival (OS, Spearman's correlation r>0.3): TRAF3IP3, C17orf85, HES1, CCNB1, SNRPD1, FGA, CALR, NRIP1, CSNK2A1, and SSB.

There were also four autoantibodies that were inversely correlated with overall survival and associated with shorter survival (Spearman's r<−0.3). SHC1, MMP3, GNAI2, and IL36RN.

Table 7 includes 19 baseline autoantibodies, which were elevated in patients, who achieve DCR following pembrolizumab treatment (SAM Score d>1.8): NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, and TEX264.

Furthermore, there were also an autoantibody signature comprising eight baseline autoantibodies that were elevated in patients with progressive disease (PD), who do not respond to pembrolizumab therapy (SAM DCR Score d<−1.8): ARRB1, DHFR, CEACAM5, MSH2, HDAC1, S100A8, LAMC1, and DFFA.

TABLE 7
Autoantibodies associated with PFS, OS and DCR
in melanoma patients treated with pembrolizumab

| | | | | | | | SAM |
|---|---|---|---|---|---|---|---|
| | | | | | | SAM | Fold-change |
| | | | R-value | R-value | R-value | Score d DCR | DCR at |
| ID | Gene ID | Gene Symbol | PFS | OS | DCR | at T0 | T0 |
| 8 | 4858 | NOVA2 | 0.40 | 0.24 | 0.35 | 2.79 | 4.48 |
| 26 | 8320 | EOMES | 0.31 | 0.15 | 0.38 | 2.31 | 4.81 |
| 23 | 6741 | SSB | 0.52 | 0.32 | 0.41 | 2.30 | 2.12 |
| 14 | 10644 | IGF2BP2 | 0.26 | 0.13 | 0.33 | 2.29 | 2.45 |
| 72 | 60 | ACTB | 0.27 | 0.09 | 0.32 | 2.19 | 2.28 |
| 73 | 4302 | MLLT6 | 0.27 | 0.09 | 0.32 | 2.19 | 2.28 |
| 22 | 6632 | SNRPD1 | 0.18 | 0.37 | 0.32 | 2.18 | 2.36 |
| 7 | 80342 | TRAF3IP3 | 0.49 | 0.41 | 0.42 | 2.18 | 1.67 |
| 4 | 55421 | C17orf85 | 0.38 | 0.38 | 0.29 | 2.12 | 2.46 |
| 19 | 3280 | HES1 | 0.26 | 0.37 | 0.33 | 1.96 | 2.25 |
| 76 | 2931 | GSK3A | 0.27 | 0.18 | 0.27 | 1.95 | 3.08 |
| 24 | 7520 | XRCC5 | 0.27 | 0.12 | 0.26 | 1.93 | 1.78 |
| 25 | 2547 | XRCC6 | 0.27 | 0.12 | 0.26 | 1.93 | 1.78 |
| 51 | 5504 | PPP1R2 | 0.23 | 0.16 | 0.32 | 1.93 | 2.33 |
| 9 | 84419 | C15orf48 | 0.24 | 0.18 | 0.34 | 1.91 | 1.58 |
| 81 | 5801 | PTPRR | 0.23 | 0.27 | 0.30 | 1.89 | 2.16 |
| 80 | 4150 | MAZ | 0.17 | 0.11 | 0.23 | 1.88 | 3.54 |
| 84 | 2316 | FLNA | 0.12 | −0.05 | 0.13 | 1.87 | 2.62 |
| 2 | 51368 | TEX264 | 0.37 | 0.23 | 0.25 | 1.87 | 2.66 |
| 55 | 6625 | SNRNP70 | 0.40 | 0.27 | 0.24 | 1.70 | 1.70 |
| 92 | 22994 | CEP131 | 0.41 | 0.25 | 0.23 | 1.68 | 1.98 |
| 56 | 6626 | SNRPA | 0.43 | 0.26 | 0.23 | 1.52 | 1.94 |
| 91 | 201161 | CENPV | 0.41 | 0.23 | 0.41 | 1.36 | 1.41 |
| 50 | 8204 | NRIP1 | 0.34 | 0.35 | 0.23 | 1.01 | 1.42 |
| 85 | 891 | CCNB1 | 0.30 | 0.37 | 0.20 | 0.99 | 1.42 |
| 53 | 22913 | RALY | 0.39 | 0.23 | 0.16 | 0.92 | 1.57 |
| 34 | 2243 | FGA | 0.17 | 0.36 | 0.22 | 0.74 | 1.13 |
| 87 | 811 | CALR | 0.14 | 0.36 | 0.20 | 0.50 | 1.16 |
| 89 | 2771 | GNAI2 | −0.39 | −0.31 | −0.02 | 0.33 | 1.09 |
| 52 | 26525 | IL36RN | −0.36 | −0.30 | −0.06 | 0.30 | 1.12 |
| 54 | 57402 | S100A14 | −0.38 | −0.15 | −0.14 | −0.12 | 0.94 |
| 20 | 4314 | MMP3 | −0.35 | −0.35 | −0.22 | −0.49 | 0.87 |
| 86 | 6464 | SHC1 | −0.20 | −0.37 | −0.24 | −0.77 | 0.86 |
| 13 | 1457 | CSNK2A1 | 0.21 | 0.35 | −0.07 | −1.05 | 0.63 |
| 82 | 1676 | DFFA | −0.17 | −0.12 | −0.40 | −1.80 | 0.60 |
| 78 | 3915 | LAMC1 | −0.03 | −0.11 | −0.29 | −1.82 | 0.41 |
| 6 | 6279 | S100A8 | −0.26 | 0.07 | −0.14 | −1.87 | 0.64 |
| 77 | 3065 | HDAC1 | −0.11 | −0.14 | −0.22 | −1.90 | 0.56 |
| 79 | 4436 | MSH2 | −0.12 | −0.11 | −0.11 | −2.05 | 0.43 |
| 75 | 1048 | CEACAM5 | −0.02 | 0.00 | −0.30 | −2.13 | 0.56 |
| 83 | 1719 | DHFR | −0.31 | −0.28 | −0.33 | −2.25 | 0.59 |
| 74 | 408 | ARRB1 | −0.28 | −0.16 | −0.33 | −2.92 | 0.31 |

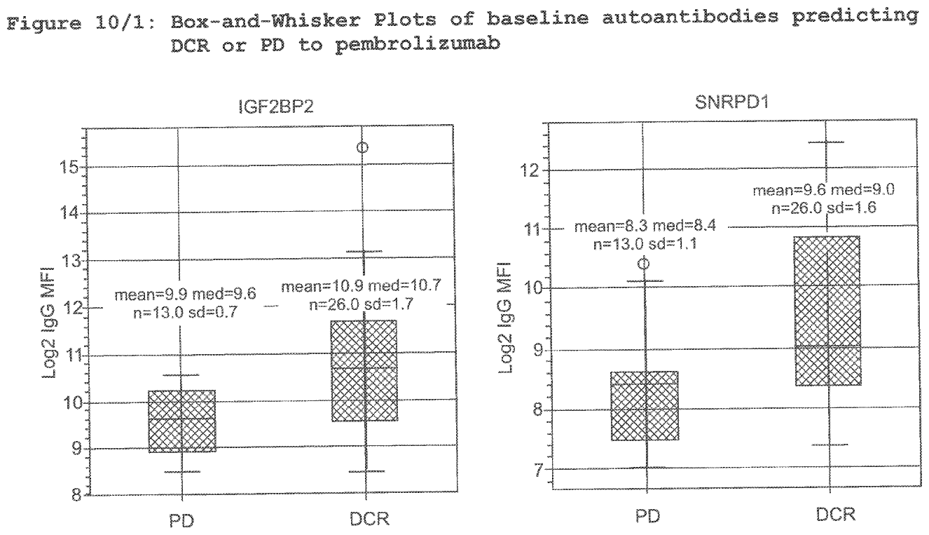

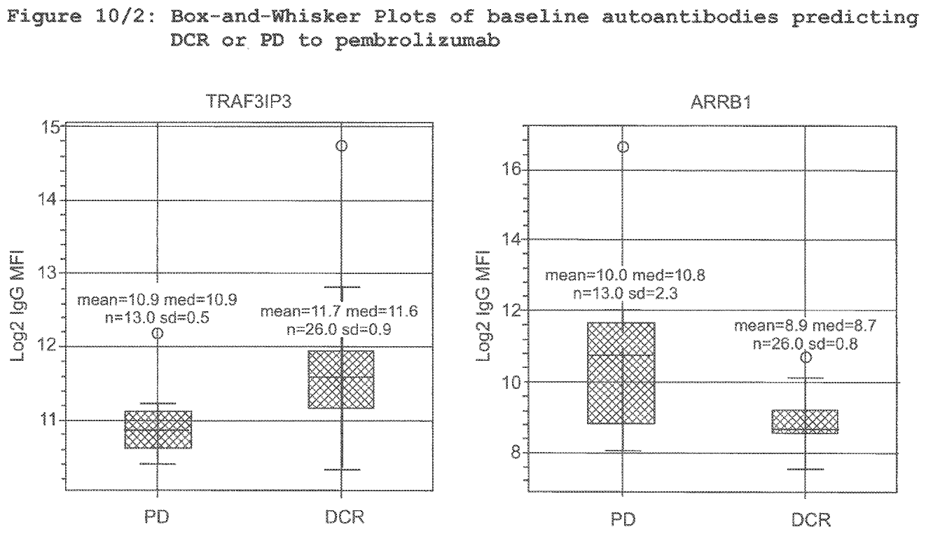

FIG. 10 shows Box-and-Whisker plots of four baseline autoantibodies targeting IGF2BP2, SNRPD1, TRAF3IP3, and ARRB1 predicting DCR or PD to pembrolizumab.

Elevated levels of baseline anti-IGFBP2 autoantibodies predict clinical response as defined as DCR in patients treated with ipilimumab and other checkpoint inhibitors (Table 3).

TRAF3IP3 is also known as “TRAF3-interacting JNK-activating modulator”, “TRAF3-interacting protein 3”, or T3JAM. TRAF3IP3 is specifically expressed in immune organs and tissues and plays a role in T and/or B cell development (Peng et al., 2015).

SNRPD1 is also known as “small nuclear ribonucleoprotein Sm D1”, snRNP core protein D1, and is core component small nuclear ribonucleoprotein (snRNP) complexes. SNRPD1 or Sm-D1 is a known autoantigen and autoantibodies against this protein are specifically associated with the autoimmune disease systemic lupus erythematosus (SLE).

ARRB1 is also known as “beta-arrestin-1”, or ARR1. ARRB1 is is critical for CD4+ T cell survival and is a factor in susceptibility to autoimmunity (Shi et al., 2007). Anti-ARRB1 antibodies are found in baseline samples of melanoma patients with clinical non-response (PD) to pembrolizumab therapy.

### Example 14: Identification of Autoantibodies Associated with irAEs in Patients Treated with Pembrolizumab

Table 8 lists 35 baseline autoantibodies that are associated with the development of irAEs in patients treated with pembrolizumab.

Twenty-seven autoantibodies show higher reactivity in baseline samples and predict the development of irAE: FADD, OGT, HSPB1, CAP2, FN1, CTSW, ATP13A2, SIGIRR, TEX264, HSPA1B, SPTB, PDCD6IP, MITF, RAPGEF3, KRT7, ERBB3, PECAM1, PPL, TONSL, ELMO2, LAMB2, BTRC, SUFU, LGALS3BP, KLKB1, EGFR, and TOLLIP.

Eight autoantibodies showed higher reactivity in the group of melanoma patients who do not develop irAE: CXXC1, SPA17, LARP1, EGLN2, RPRM, WHSC1L1, MIF, and S100A8.

TABLE 8
Baseline autoantibodies predicting irAEs in
melanoma patients treated with pembrolizumab

| | | | SAM | SAM |
|---|---|---|---|---|
| | Gene | Gene | Score.d.iRAE | Fold.Change |
| ID | ID | Symbol | at T0 | irAE at T0 |
| 99 | 8772 | FADD | 3.08 | 2.83 |
| 54 | 8473 | OGT | 2.94 | 2.53 |
| 46 | 3315 | HSPB1 | 2.67 | 3.05 |
| 13 | 10486 | CAP2 | 2.59 | 2.35 |
| 153 | 2335 | FN1 | 2.55 | 2.40 |
| 94 | 1521 | CTSW | 2.43 | 2.30 |
| 90 | 23400 | ATP13A2 | 2.39 | 2.99 |
| 158 | 59307 | SIGIRR | 2.37 | 3.26 |
| 7 | 51368 | TEX264 | 2.31 | 3.14 |
| 20 | 3304 | HSPA1B | 2.14 | 2.37 |
| 21 | 6710 | SPTB | 2.14 | 2.37 |
| 27 | 10015 | PDCD6IP | 2.13 | 2.14 |
| 156 | 4286 | MITF | 2.13 | 2.48 |
| 83 | 10411 | RAPGEF3 | 2.12 | 3.48 |
| 154 | 3855 | KRT7 | 2.11 | 2.81 |
| 121 | 2065 | ERBB3 | 2.05 | 1.74 |
| 55 | 5175 | PECAM1 | 2.03 | 1.86 |
| 157 | 5493 | PPL | 2.01 | 2.12 |
| 162 | 4796 | TONSL | 1.98 | 2.44 |
| 142 | 63916 | ELMO2 | 1.89 | 1.98 |
| 155 | 3913 | LAMB2 | 1.89 | 2.25 |
| 151 | 8945 | BTRC | 1.87 | 2.05 |
| 160 | 51684 | SUFU | 1.87 | 1.81 |
| 52 | 3959 | LGALS3BP | 1.84 | 1.66 |
| 50 | 3818 | KLKB1 | 1.83 | 1.48 |
| 152 | 1956 | EGFR | 1.81 | 2.07 |
| 161 | 54472 | TOLLIP | 1.81 | 1.79 |
| 97 | 30827 | CXXC1 | −1.83 | 0.50 |
| 159 | 53340 | SPA17 | −1.85 | 0.43 |
| 51 | 23367 | LARP1 | −1.85 | 0.55 |
| 77 | 112398 | EGLN2 | −1.86 | 0.69 |
| 86 | 56475 | RPRM | −1.90 | 0.52 |
| 87 | 54904 | WHSC1L1 | −1.94 | 0.53 |
| 10 | 4282 | MIF | −2.11 | 0.54 |
| 6 | 6279 | S100A8 | −2.19 | 0.61 |

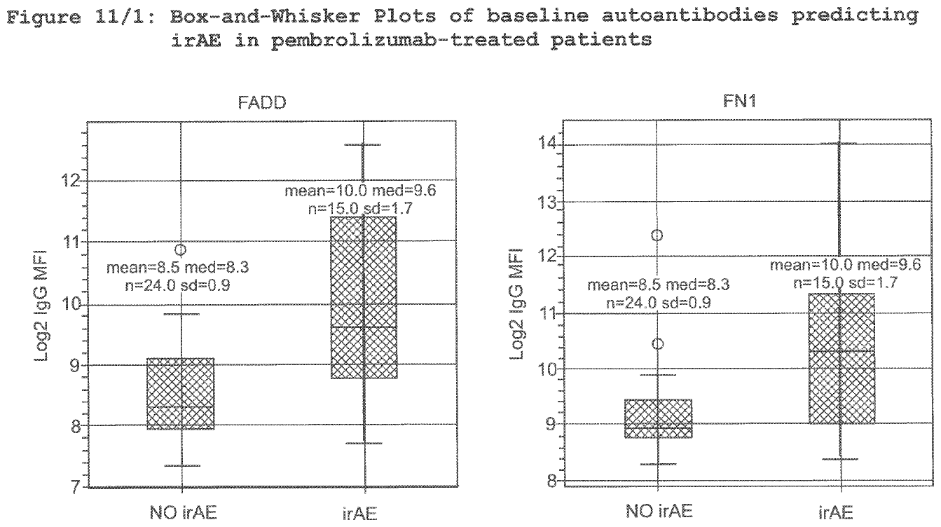

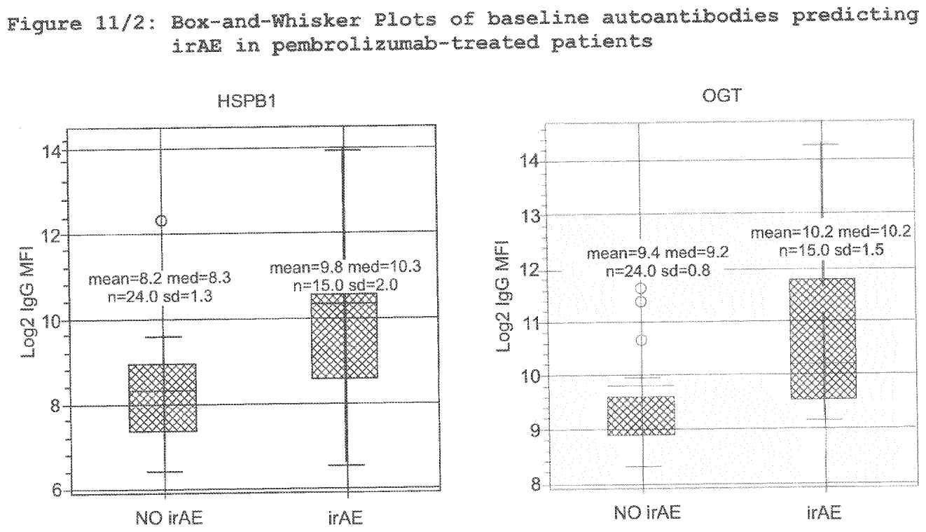

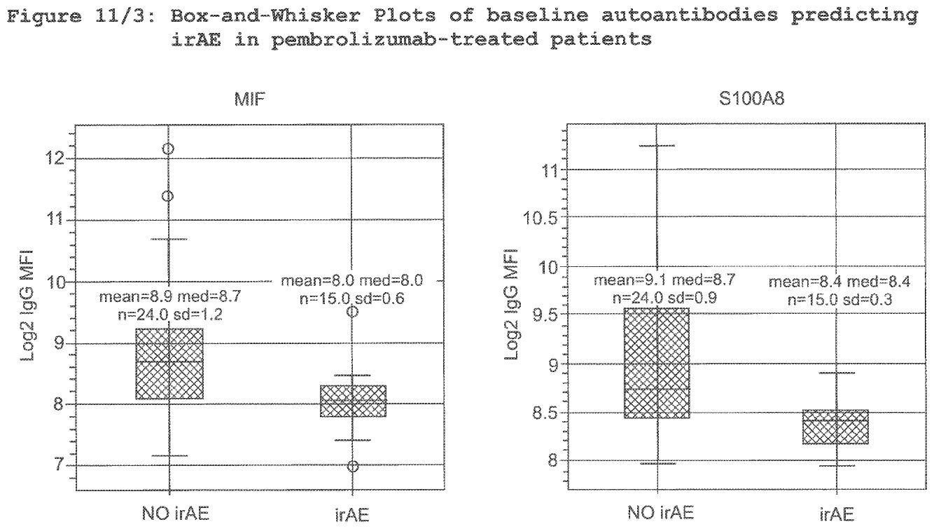

FIG. 11 shows Box-and-Whisker plots of four baseline autoantibody targets, FADD, FN1, HSPB1, and OGT, predicting irAE in pembrolizumab-treated patients.

Elevated autoantibodies directed against the pro-inflammatory cytokines S100A8 and MIF were found in melanoma patients who do not develop irAEs following pembrolizumab treatment.

MIF is also known as “Macrophage migration inhibitory factor (EC:5.3.2.1)”, “Glycosylation-inhibiting factor”, L-dopachrome tautomerase (EC:5.3.3.12), “Phenylpyruvate tautomerase”, GLIF, or MIF. MIF is a broad-spectrum proinflammatory cytokine, which plays a role in inflammatory and autoimmune diseases, but also has tumor-promoting effects (Kindt et al., 2016).

S100A8 is also known as “Protein S100-A8”, “Calgranulin-A”, “Calprotectin L1L subunit”, “Migration inhibitory factor-related protein 8”, CFAG, or MRP8. S100A8 is a calcium- and zinc-binding protein, which plays a prominent role in the regulation of inflammatory processes and immune response. In many cancer types including melanoma, overexpression of 100A8 contributes to the growth, metastasis, angiogenesis and immune evasion of tumors (Bresnick et al., 2015). Elevated levels of anti-S100A8 antibodies were also found in melanoma patients with progressive disease following pembrolizumab.

FADD is an also known as “FAS-associated death domain protein”, “Growth-inhibiting gene 3 protein”, “Mediator of receptor induced toxicity”, MORT1, or GIG3. FADD is an adaptor protein that bridges members of the tumor necrosis factor receptor superfamily, such as the Fas-receptor, to procaspases 8 and 10 to form the death-inducing signaling complex (DISC) during apoptosis. FADD has an important role in apoptosis, cell cycle regulation and cell survival, so that it can exert both tumor-suppressive and tumor-promoting roles. FADD is also is involved in inflammatory processes in autoimmune diseases (Cuda et al., 2016).

FN1 is also known as “Fibronectin”, “Cold-insoluble globulin”, or CIG. Fibronectin is a component of the extracellular matrix that plays a role in wound healing. In cancer, fibronectin promotes tumor growth/survival and resistance to therapy.

HSBP1 is also known as “Heat shock protein beta-1”, “28 kDa heat shock protein”, “Estrogen-regulated 24 kDa protein”, “Heat shock 27 kDa protein”, HSP27, or HSP28. HSBP1 is a multifunctional protein, which acts as a protein chaperone and an antioxidant. In cancer, HSP27 plays a role in the inhibition of apoptosis.

OGT is also known as “UDP-N-acetylglucosamine-peptide N-acetylglucosaminyltransferase 110 kDa subunit (EC:2.4.1.2554)”, or “O-GlcNAc transferase subunit p110”. OGT catalyzes the O-GlcNAcylation of a number of nuclear and cytoplasmic proteins thereby modulating cellular development and signaling pathways. Many cancer types display elevated O-GlcNAcylation and aberrant expression of OGT linking metabolism to invasion and metastasis (Ferrer et al., 2016).

### Example 15: Development of Biomarkers for Predicting the Risk to Develop an irAE

Multi-cohort metastatic melanoma samples for developing biomarker panels for irAE were obtained as follows.

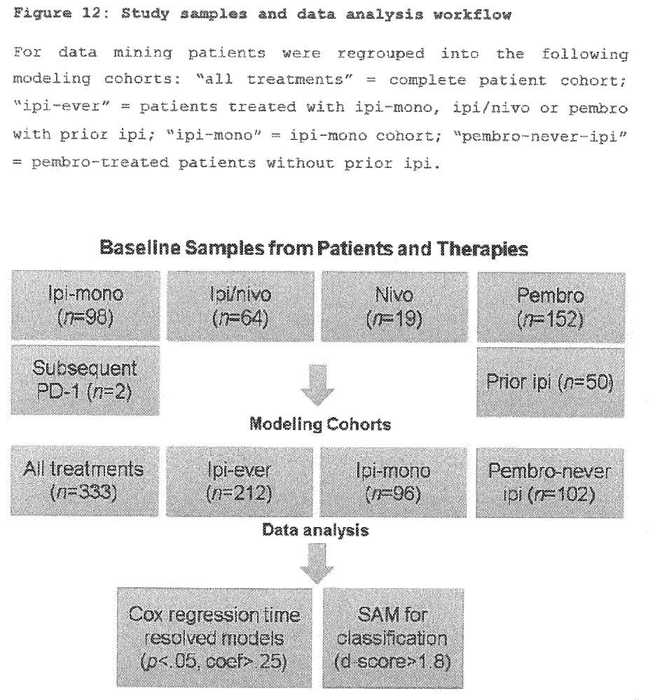

Serum samples from 333 metastatic melanoma patients were collected at 5 European cancer centers prior to treatment with the following therapeutic monoclonal antibodies ipilimumab (ipi, anti-CTLA-4), nivolumab (nivo, anti-PD-1), pembrolizumab (pembro, anti-PD-1), or ipilimumab with nivolumab combination therapy (FIG. 12). Serum samples were analyzed using a cancer immunotherapy antigen array (FIG. 1) comprising 832 antigens and were used to develop autoantibody biomarker panels for irAE and its subtype colitis.

All individuals provided written informed consent and the study was approved by the respective Ethics Committees. Patient data were provided including demographics (age, gender), treatment, date of therapy start, and best response (RECIST 1.1. criteria. Furthermore, irAEs were recorded including onset date and grade. As the risk for colitis might influence treatment choice in metastatic melanoma, namely the decision for anti-PD-1 monotherapy or ipi/nivo combination treatment we included colitis as an irAE of special interest.

The 333 included patients had a median age of 61 years, 38% were female. Overall, 103 patients (31%) developed irAEs including 44 patients with colitis (13%). Of the 98 patients who were treated with ipi monotherapy, 34 patients (35%) experienced an irAE of any grade and type, and 17 (17%) had colitis. Of 152 patients who were treated with pembrolizumab (pembro), 37 (24%) developed an irAE of any grade, 11 patients colitis (7%). 47 (31%) of pembro-treated patients had received ipi before, 14 patients (38%) of the irAE group and six (55%) of the colitis group. 64 patients were treated with ipi/nivo combination therapy of which 28 (44%) had any type of irAE and 15 had colitis (23%).

Statistical analysis for predicting the risk to develop an irAE was undertaken as follows.

To encode the different types of checkpoint inhibitors (anti-CTLA-4 and anti-PD-1) as a factor, we produced 5 modeling cohorts for data analysis (FIG. 12). We generated two CPI monotherapy groups (“ipi-mono” and pembro-never-ipi), which only include patients who received no other than the current CPI, and one combination therapy group “ipi/nivo”. Patients treated with ipi-mono, ipi-nivo or who have previously been treated with ipi were combined into the “ipi-ever” group. All 333 patients were also jointly investigated in the “all-treatments” analysis group.

To identify the most relevant biomarkers, we used a combination of linear and nonlinear data mining methods, which complement each other for feature selection. Significance Analysis of Microarrays (SAM) was used to compare patients according to the class label irAE or colitis. We used 1,000 permutations in a multiple testing approach for each autoantibody feature to ensure robust modeling. Feature ranking was achieved using the absolute value of the output d-score. Candidate biomarkers were included in the set of final biomarker candidates using a threshold of the SAM score |d|>1.8.

As a second approach for feature selection, Cox regression analysis was performed to investigate if pre-treatment autoantibody levels are related to the hazard ratio of an event using the R's survival package. For Cox regression the treatment regime was included using the three membership classes (PD1, CTLA4, PD1+CTLA4) as covariate factors. Within time-to-event, all relevant treatments with respect to the presence of PD1 or CTLA4 inhibition were considered in the covariate factor. The models were created in a one factor bottom up multiple testing approach (i.e. each biomarker was investigated one after another). For feature selection, we utilized the unadjusted p-value (p<0.05) of the Cox regression in combination with a minimum coefficient (coef>0.25). “Last contact” (and “death” for irAE and colitis) were taken to censor the data to acknowledge data points from patients dropped out.

Kaplan Meier curves were calculated in combination with the Logrank test (using “survdiff” from R's survival package) for the same groups as for Cox regression, except for the all treatments group (http://www.sthda.com/english/rpkgs/survminer). Time-to-event was recorded starting at CPI therapy. The autoantibody data were dichotomized into autoantibody high versus low using the mean MFI value+1 SD of the healthy control sera as a marker-specific threshold.

As a complementary approach for feature ranking, Random Forests (RF) were calculated. We used a modification of the two-class classification method described by [10] using the “Tree-ensemble-learner” from KNIME. A number of 10,000 different models were generated. The tree depth was limited to 4 to investigate small panels with shallow trees, minimum split node size was 10 with minimum child node size of 5. The fraction of training data used for each model was 80% and attribute sampling was sampling a square root of total attributes combined with resampling for each tree node. Feature ranking was performed creating a score of the relative marker contribution for the first two levels of each tree.

Final feature ranking was performed by ranking markers according to their appearance in the respective tests. Final marker selection was performed to yield markers, which were above threshold in at least three tests.

Table 9 shows the top 47 autoantibodies predicting irAE or colitis.

The predictive autoantibody signature comprises the following antigen specificities:

SUMO2, MAGED2, PIAS3, MITF, GRP, AP2B1, PRKCI, BTBD2, AKT2, UBE2Z, L1CAM, LAMC1, GABARAPL2, RPLP0, SDCBP, AP1S1, CFB, FGA, IL3, IL4R, AMPH, LEPR, TP53, GPHN, IL23A, BAG6, BICD2, TMEM98, KDM4A, UBTF, CASP8, PCDH1, RELT, SPTBN1, MUM1, RPLP2, KRT7, FN1, MAGEB4, CTSW, NCOA1, MIF, SPA17, FGFR1, KRT19, TPM2, ATG4D.

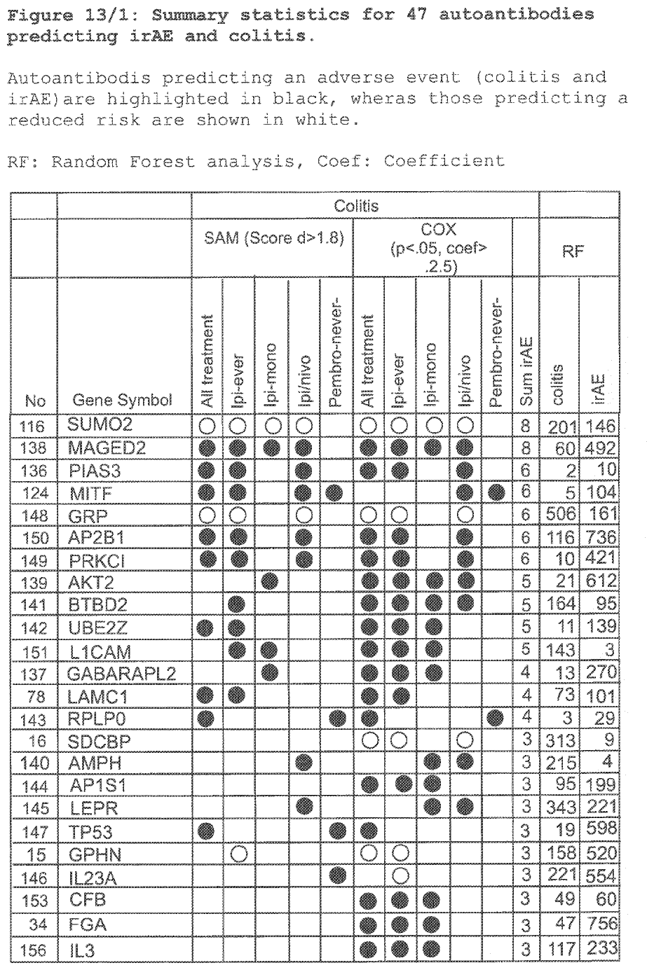

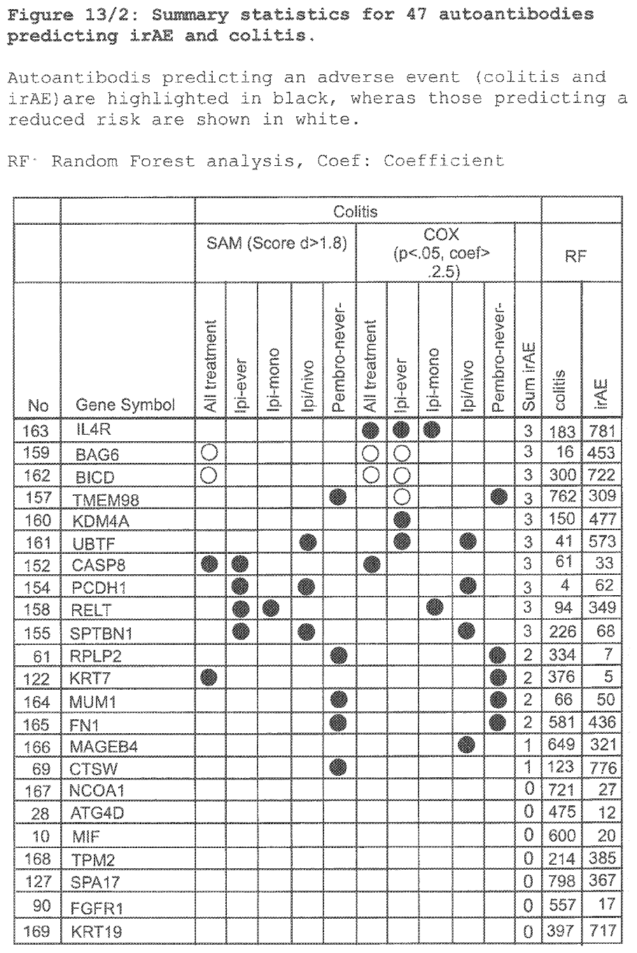

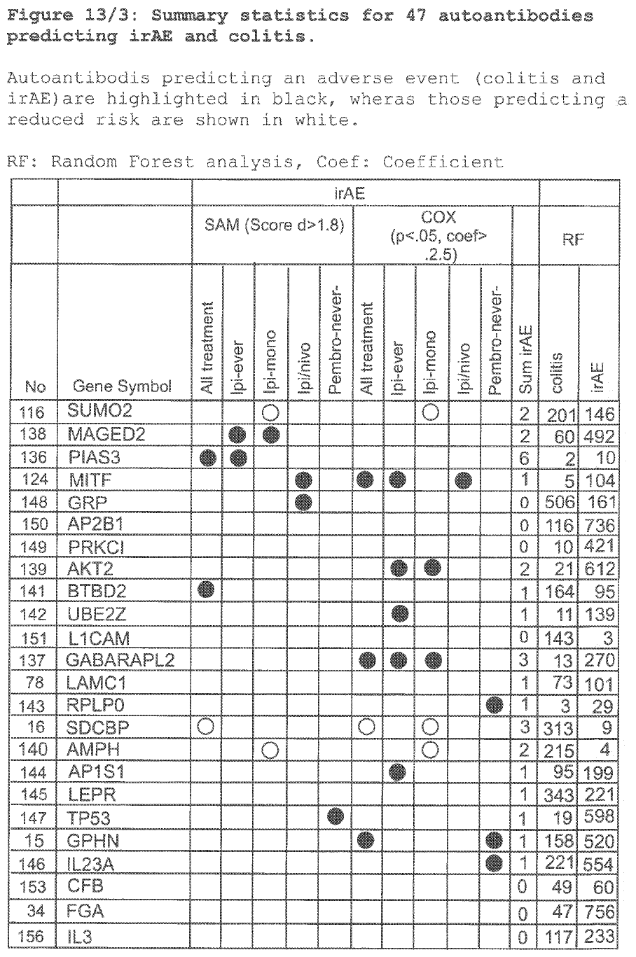

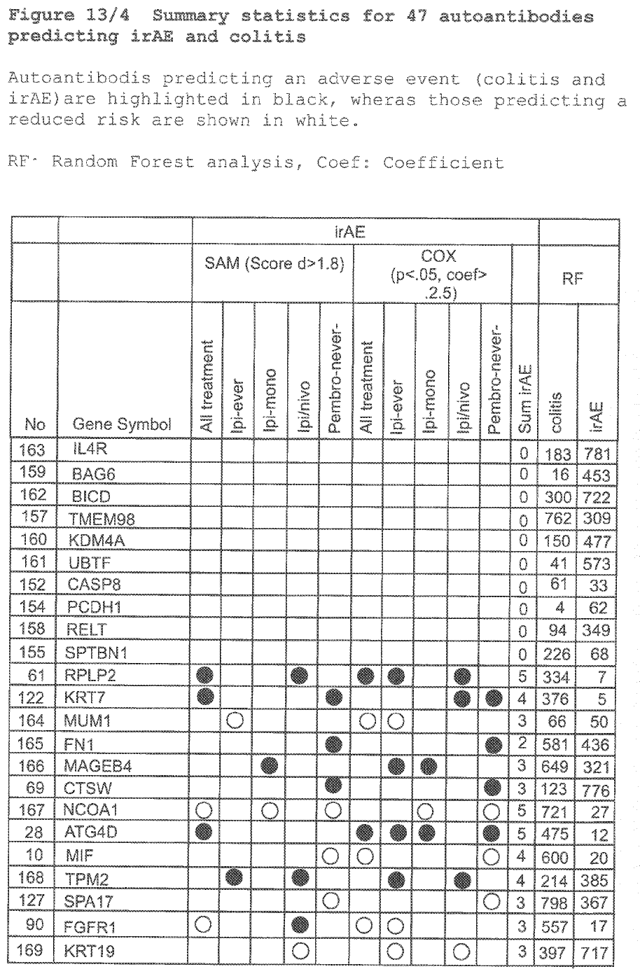

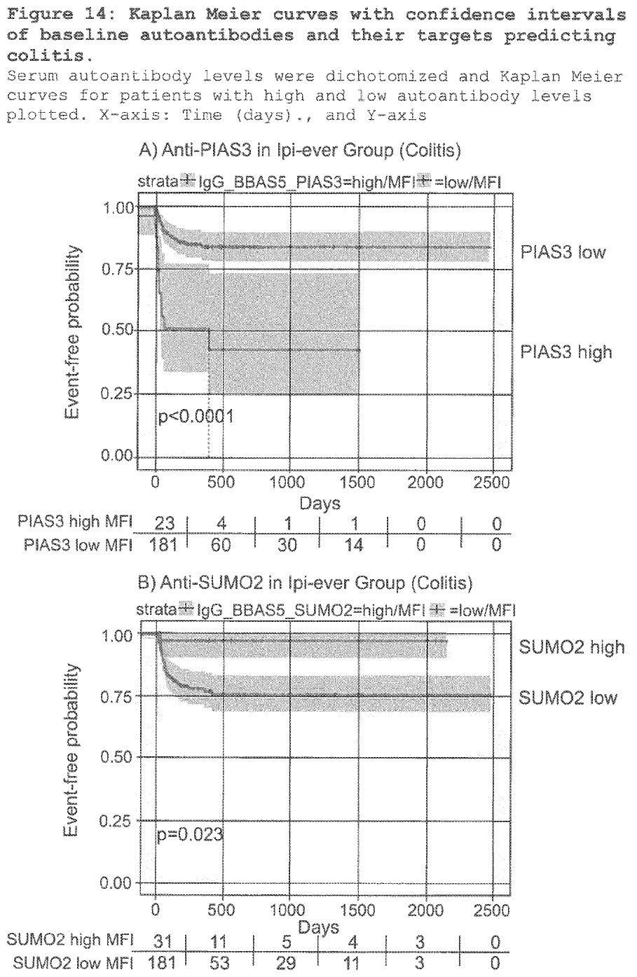

FIG. 13 summarizes the statistical test results and highlights autoantibodies that positively (black circles) or negatively (white circles) predict irAE or colitis.

TABLE 9
List of top 47 marker predicting irAE or colitis
The thresholds were: SAM analysis (Score d > 1.8) and Cox
regression analysis (p < 0.05, coefficient 0 > .25) in any of the
modeling cohorts.

| | | Gene | | | | | |
|---|---|---|---|---|---|---|---|
| | | Symbol | | | | | |
| | | (and | | | | | |
| | | Exemplary | | | | | |
| | Gene | Antigen | | Colitis | | IRAE | |
| No | ID | Sequence) | Gene name | SAM | Cox | SAM | Cox |
| 10 | 4282 | MIF | Macrophage migration | | | x | x |
| | | | inhibitory factor | | | | |
| 15 | 10243 | GPHN | Gephyrin | x | x | | x |
| 16 | 6386 | SDCBP | Syntenin-1 | | x | x | x |
| 28 | 84971 | ATG4D | Cysteine protease | | | x | x |
| | | | ATG4D | | | | |
| 34 | 2243 | FGA | Fibrinogen alpha | | x | | |
| | | | chain | | | | |
| 61 | 6181 | RPLP2 | 60S acidic ribosomal | x | x | x | x |
| | | | protein P2 | | | | |
| 69 | 1521 | CTSW | Cathepsin W | x | | x | x |
| 78 | 3915 | LAMC1 | Laminin subunit | x | x | x | |
| | | | gamma-1 | | | | |
| 90 | 2260 | FGFR1 | Fibroblast growth | | | x | x |
| | | | factor receptor 1 | | | | |
| | | | (CD331) | | | | |
| 116 | 6613 | SUMO2 | Small ubiquitin- | x | x | x | x |
| | | | related modifier 2 | | | | |
| 122 | 3855 | KRT7 | Cytokeratin-7 | x | x | x | x |
| 124 | 4286 | MITF | Microphthalmia- | x | x | x | |
| | | | associated | | | | |
| | | | transcription factor | | | | |
| 127 | 53340 | SPA17 | Sperm surface | | | x | x |
| | | | protein Sp17 (CT22) | | | | |
| 136 | 10401 | PIAS3 | E3 SUMO-protein | x | x | x | x |
| | | (SEQ ID | ligase PIAS3 | | | | |
| | | NO: 136) | | | | | |
| 137 | 11345 | GABARAPL2 | (GABA(A) receptor- | x | x | | x |
| | | (SEQ ID | associated protein- | | | | |
| | | NO: 137) | like 2 | | | | |
| 138 | 10916 | MAGED2 | Melanoma-associated | x | x | x | |
| | | (SEQ ID | antigen D2 | | | | |
| | | NO: 138) | | | | | |
| 139 | 208 | AKT2 | RAC-beta | x | x | | x |
| | | (SEQ ID | serine/threonine- | | | | |
| | | NO: 139) | protein kinase | | | | |
| 140 | 273 | AMPH | Amphiphysin | x | x | x | x |
| | | (SEQ ID | | | | | |
| | | NO: 140) | | | | | |
| 141 | 55643 | BTBD2 | BTB/POZ domain- | x | x | x | |
| | | (SEQ ID | containing protein 2 | | | | |
| | | NO: 141) | | | | | |
| 142 | 65264 | UBE2Z | Ubiquitin- | x | x | | x |
| | | (SEQ ID | conjugating enzyme | | | | |
| | | NO: 142) | E2 Z | | | | |
| 143 | 6175 | RPLP0 | 60S acidic ribosomal | x | x | | x |
| | | (SEQ ID | protein P0 | | | | |
| | | NO: 143) | | | | | |
| 144 | 1174 | AP1S1 | AP-1 complex subunit | | x | | x |
| | | (SEQ ID | sigma-1A | | | | |
| | | NO: 144) | | | | | |
| 145 | 3953 | LEPR | Leptin receptor | x | x | x | |
| | | (SEQ ID | (CD295) | | | | |
| | | NO: 145) | | | | | |
| 146 | 51561 | IL23A | Interleukin-23 | x | x | | x |
| | | (SEQ ID | subunit alpha (IL- | | | | |
| | | NO: 146) | 23p19) | | | | |
| 147 | 7157 | TP53 | Cellular tumor | x | x | x | |
| | | (SEQ ID | antigen p53 | | | | |
| | | NO: 147) | | | | | |
| 148 | 2922 | GRP | Gastrin-releasing | x | x | | |
| | | (SEQ ID | peptide | | | | |
| | | NO: 148) | | | | | |
| 149 | 5584 | PRKCI | Protein kinase C | x | x | | |
| | | (SEQ ID | iota type | | | | |
| | | NO: 149) | | | | | |
| 150 | 163 | AP2B1 | AP-2 complex subunit | x | x | | |
| | | (SEQ ID | beta | | | | |
| | | NO: 150) | | | | | |
| 151 | 3897 | L1CAM | Neural cell adhesion | x | x | | |
| | | (SEQ ID | molecule L1 (CD171) | | | | |
| | | NO: 151) | | | | | |
| 152 | 841 | CASP8 | Caspase-8 | x | x | | |
| | | (SEQ ID | | | | | |
| | | NO: 152) | | | | | |
| 153 | 629 | CFB | Complement factor B | | x | | |
| | | (SEQ ID | | | | | |
| | | NO: 153) | | | | | |
| 154 | 5097 | PCDH1 | Protocadherin-1 | x | x | | |
| | | (SEQ ID | | | | | |
| | | NO: 154) | | | | | |
| 155 | 6711 | SPTBN1 | Spectrin beta chain | x | x | | |
| | | (SEQ ID | | | | | |
| | | NO: 155) | | | | | |
| 156 | 3562 | IL3 | Interleukin-3 | | x | | |
| | | (SEQ ID | | | | | |
| | | NO: 156) | | | | | |
| 157 | 26022 | TMEM98 | Transmembrane | x | x | | |
| | | (SEQ ID | protein 98 (Protein | | | | |
| | | NO: 157) | TADA1) | | | | |
| 158 | 84957 | RELT | Tumor necrosis | x | x | | |
| | | (SEQ ID | factor receptor | | | | |
| | | NO: 158) | superfamily member | | | | |
| | | | 19L | | | | |
| 159 | 7917 | BAG6 | Large proline-rich | x | x | | |
| | | (SEQ ID | protein BAG6 | | | | |
| | | NO: 159) | | | | | |
| 160 | 9682 | KDM4A | Lysine-specific | x | x | | |
| | | (SEQ ID | demethylase 4A | | | | |
| | | NO: 160) | | | | | |
| 161 | 7343 | UBTF | Nucleolar | x | x | | |
| | | (SEQ ID | transcription factor | | | | |
| | | NO: 161) | 1 (Autoantigen NOR- | | | | |
| | | | 90) | | | | |
| 162 | 23299 | BICD2 | Protein bicaudal D | x | x | | |
| | | (SEQ ID | homolog 2 | | | | |
| | | NO: 162) | | | | | |
| 163 | 3566 | IL4R | Interleukin-4 | | x | | |
| | | (SEQ ID | receptor subunit | | | | |
| | | NO: 163) | alpha (CD124) | | | | |
| 164 | 84939 | MUM1 | Mutated melanoma- | x | x | x | x |
| | | (SEQ ID | associated antigen 1 | | | | |
| | | NO: 164) | | | | | |
| 165 | 2335 | FN1 | Fibronectin | x | x | x | x |
| | | (SEQ ID | | | | | |
| | | NO: 165) | | | | | |
| 166 | 4115 | MAGEB4 | Melanoma-associated | | x | x | x |
| | | (SEQ ID | antigen B4 | | | | |
| | | NO: 166) | | | | | |
| 167 | 8648 | NCOA1 | Nuclear receptor | | | x | x |
| | | (SEQ ID | coactivator 1 | | | | |
| | | NO: 167) | | | | | |
| 168 | 7169 | TPM2 | Tropomyosin beta | | | x | x |
| | | (SEQ ID | chain | | | | |
| | | NO: 168) | | | | | |
| 169 | 3880 | KRT19 | Cytokeratin-19 | | | x | x |
| | | (SEQ ID | | | | | |
| | | NO: 169) | | | | | |

Association rule mining was performed using the software Natto Ef Prime Inc. (Japan) and network graphs with corresponding associations were created. Autoantibody intensity data were categorized into 3 categories (low, medium and high intensity). We computed a description score as an index, which represents the proportion of uncertainty in Y that X can explain for each edge in the network as mutual information. We selected irAE and colitis as targets to highlight the relevant attributes, which have the highest description scores (mutual information) in the model.

### Example 16: Exploration of an Autoantibody Signature for Prediction of Colitis

This analysis yielded 34 autoantibodies for predicting colitis, which were found in three group comparisons as shown in FIG. 13.

The results of the Cox regression analysis and the associated hazard risk for developing an irAE in patients with high autoantibody levels is shown in Table 10 for the autoantibody signature predicting irAE and colitis.

The 35 autoantibodies comprise the following antigen specificities: SUMO2, MAGED2, PIAS3, MITF, GRP, AP2B1, PRKCI, BTBD2, AKT2, UBE2Z, L1CAM, LAMC1, GABARAPL2, RPLP0, SDCBP, AP1S1, CFB, FGA, IL3, IL4R, AMPH, LEPR, TP53, GPHN, IL23A, BAG6, BICD2, TMEM98, KDM4A, UBTF, CASP8, PCDH1, RELT, SPTBN1, MUM1.

30 of the 34 antigens were identified in the ipi-ever group, demonstrating a strong association of anti-CTLA-4 therapy with the development of colitis (FIG. 13, Table 10). However, there were also differences seen in autoantibody patterns between ipi-mono and ipi/nivo combination therapy. For example, UBE2Z, L1CAM, GABARAPL2, CFB, IL3, RELT, FGA, and IL4R predict colitis in the ipi-mono group, whereas PIAS3, SUMO2, MITF, GRP, PRKCI, AP2B1, SDCBP, PDCH1, SPTBN1, and UBTF were predictive in the ipi/nivo cohort.

The markers with the highest score for predicting colitis were MAGED2, PIAS3, MITF, PRKC1, and A2B1 (FIG. 13). Two high scoring markers predicted a reduced risk to develop colitis, which were SUMO2, and GRP.

The marker with the highest score for predicting colitis was MAGED2 with significant associations found for the all treatment (HR 1.35, p=0.002), ipi-ever (HR 1.36, p=0.0012), CLTA4-mono (HR 1.48, p=0.024), and ipi/nivo group (HR 1.31, p=0.036). The marker with the smallest p-value in the ipi-ever group was PIAS3 with significant associations for the all treatment (HR 1.42, p=0.00005), ipi-ever (HR 1.46, p=0.000009), and ipi/nivo group (HR 1.52, p=0.0004). FIG. 13 shows the Kaplan-Meier curve for PIAS3.

Higher levels of SUMO2 autoantibodies predicted a lower risk to develop colitis in the all treatment (HR 0.53, p=0.0022), ipi-ever (HR 0.51, p=0.0026), ipi-mono (HR 0.32, p=0.0012), and ipi/nivo group (HR 0.5, p=0.049).

FIG. 13 shows examples of Kaplan-Meier curves for PIAS3 and SUM02.

TABLE 10
Results of Cox regression analysis of autoantibodies predicting colitis

| Gene | All Treatments | | Ipi Ever | | Ipi Mono | | Ipi/Nivo | | Pembro Never Ipi | |
|---|---|---|---|---|---|---|---|---|---|---|
| Symbol | P-value | HR | P-value | HR | P-value | HR | P-value | HR | P-value | HR |
| AKT2 | 0.0006 | 1.75 | 0.0008 | 1.80 | 0.0006 | 2.25 | 0.0307 | 1.96 | 0.1640 | 1.95 |
| AMPH | 0.3562 | 1.09 | 0.3764 | 1.09 | 0.0449 | 0.75 | 0.0020 | 1.70 | 0.4250 | 1.26 |
| AP1S1 | 0.0344 | 1.49 | 0.0124 | 1.58 | 0.0451 | 1.62 | 0.2169 | 1.45 | 0.2509 | 0.31 |
| AP2B1 | 0.0040 | 1.33 | 0.0092 | 1.30 | 0.5221 | 1.19 | 0.0165 | 1.31 | 0.2165 | 1.74 |
| ATG4D | 0.9413 | 1.02 | 0.6092 | 1.14 | 0.2384 | 1.44 | 0.7893 | 1.15 | 0.3340 | 0.36 |
| BAG6 | 0.0386 | 0.71 | 0.0430 | 0.69 | 0.1749 | 0.69 | 0.2718 | 0.76 | 0.5946 | 0.79 |
| BICD2 | 0.0128 | 0.58 | 0.0354 | 0.63 | 0.2746 | 0.67 | 0.0611 | 0.49 | 0.1658 | 0.30 |
| BTBD2 | 0.0052 | 1.37 | 0.0015 | 1.41 | 0.0205 | 1.47 | 0.0369 | 1.34 | 0.3489 | 0.55 |
| CASP8 | 0.0069 | 1.30 | 0.0175 | 1.28 | 0.2020 | 1.21 | 0.4043 | 1.23 | 0.2672 | 1.38 |
| CFB | 0.0296 | 2.00 | 0.0147 | 2.16 | 0.0029 | 2.73 | 0.6696 | 1.33 | 0.5528 | 0.41 |
| CTSW | 0.4987 | 1.11 | 0.6748 | 0.92 | 0.3918 | 0.75 | 0.3826 | 0.74 | 0.2066 | 1.38 |
| FGA | 0.0430 | 1.56 | 0.0135 | 1.72 | 0.0137 | 2.45 | 0.3334 | 1.46 | 0.1526 | 0.18 |
| FGFR1 | 0.1905 | 0.72 | 0.1950 | 0.71 | 0.4259 | 0.78 | 0.3725 | 0.61 | 0.8267 | 0.84 |
| FN1 | 0.2507 | 1.20 | 0.9314 | 1.02 | 0.8925 | 0.95 | 0.5645 | 0.78 | 0.0356 | 2.05 |
| GABARAPL2 | 0.0140 | 1.62 | 0.0036 | 1.84 | 0.0035 | 2.09 | 0.5476 | 0.70 | 0.5252 | 0.58 |
| GPHN | 0.0203 | 0.57 | 0.0062 | 0.48 | 0.0539 | 0.49 | 0.0583 | 0.34 | 0.4375 | 1.45 |
| GRP | 0.0025 | 0.71 | 0.0048 | 0.71 | 0.0696 | 0.69 | 0.0473 | 0.66 | 0.2100 | 0.50 |
| IL23A | 0.6077 | 0.90 | 0.0333 | 0.55 | 0.0928 | 0.41 | 0.1936 | 0.60 | 0.0232 | 1.86 |
| IL3 | 0.0258 | 1.31 | 0.0361 | 1.31 | 0.0351 | 1.56 | 0.7551 | 0.91 | 0.0928 | 2.41 |
| IL4R | 0.0444 | 1.74 | 0.0398 | 1.77 | 0.0283 | 2.15 | 0.1662 | 2.15 | 0.7443 | 0.72 |
| KDM4A | 0.0085 | 1.39 | 0.0027 | 1.45 | 0.4092 | 1.35 | 0.3540 | 1. 18 | 0.5818 | 0.68 |
| KRT19 | 0.5878 | 0.93 | 0.4218 | 0.88 | 0.7679 | 0.92 | 0.0944 | 0.58 | 0.4050 | 1.31 |
| KRT7 | 0.0166 | 1.25 | 0.0620 | 1.21 | 0.8772 | 1.03 | 0.1078 | 1.27 | 0.0360 | 1.74 |
| L1CAM | 0.0482 | 1.29 | 0.0171 | 1.38 | 0.0082 | 1.65 | 0.2069 | 1.36 | 0.4026 | 0.63 |
| LAMC1 | 0.0017 | 1.31 | 0.0015 | 1.33 | 0.1737 | 1.19 | 0.2561 | 1.18 | 0.8534 | 1.05 |
| LEPR | 0.1914 | 1.11 | 0.0730 | 1.17 | 0.0397 | 1.29 | 0.0357 | 1.31 | 0.6056 | 0.86 |
| MAGEB4 | 0.3248 | 1.11 | 0.1395 | 1.18 | 0.5536 | 1.11 | 0.0237 | 1.36 | 0.4077 | 0.68 |
| MAGED2 | 0.0020 | 1.35 | 0.0012 | 1.36 | 0.0241 | 1.38 | 0.0361 | 1.31 | 0.5827 | 0.65 |
| MIF | 0.0902 | 0.67 | 0.1591 | 0.71 | 0.4832 | 0.81 | 0.2980 | 0.57 | 0.3325 | 0.53 |
| MITF | 0.0018 | 1.28 | 0.0217 | 1.22 | 0.7467 | 0.95 | 0.0007 | 1.47 | 0.0484 | 1.54 |
| MUM1 | 0.3432 | 1.13 | 0.6524 | 0.93 | 0.4233 | 0.80 | 0.3924 | 1.19 | 0.0098 | 1.71 |
| NCOA1 | 0.0912 | 0.85 | 0.1552 | 0.87 | 0.1005 | 0.75 | 0.4389 | 1.11 | 0.1812 | 0.57 |
| PCDH1 | 0.0916 | 1.13 | 0.0204 | 1.20 | 0.2108 | 1.14 | 0.0147 | 1.35 | 0.2909 | 0.70 |
| PIAS3 | 0.0000 | 1.42 | 0.0000 | 1.46 | 0.2308 | 1.22 | 0.0004 | 1.52 | 0.8245 | 0.88 |
| PRKCI | 0.0002 | 1.39 | 0.0001 | 1.41 | 0.0601 | 1.35 | 0.0014 | 1.44 | 0.5171 | 0.64 |
| RELT | 0.0249 | 1.19 | 0.0116 | 1.23 | 0.0289 | 1.29 | 0.7263 | 0.92 | 0.9069 | 0.96 |
| RPLP0 | 0.0102 | 1.40 | 0.2227 | 1.22 | 0.0591 | 1.36 | 0.6500 | 0.79 | 0.0007 | 3.26 |
| RPLP2 | 0.1858 | 1.22 | 0.5715 | 1.10 | 0.3298 | 1.32 | 0.8022 | 1.06 | 0.0132 | 2.43 |
| SDCBP | 0.0068 | 0.44 | 0.0304 | 0.52 | 0.3783 | 0.73 | 0.0271 | 0.27 | 0.1167 | 0.24 |
| SPA17 | 0.8799 | 1.01 | 0.7909 | 1.02 | 0.2891 | 0.85 | 0.3274 | 1.13 | 0.9692 | 1.01 |
| SPTBN1 | 0.0460 | 1.21 | 0.0245 | 1.23 | 0.7310 | 1.05 | 0.0218 | 1.39 | 0.4612 | 0.79 |
| SUMO2 | 0.0022 | 0.53 | 0.0026 | 0.51 | 0.0093 | 0.32 | 0.0488 | 0.50 | 0.9235 | 0.96 |
| TMEM98 | 0.1323 | 0.85 | 0.0207 | 0.73 | 0.0516 | 0.64 | 0.5518 | 0.88 | 0.0107 | 1.77 |
| TP53 | 0.0118 | 1.29 | 0.4205 | 1.12 | 0.3735 | 1.15 | 0.5922 | 0.82 | 0.1227 | 1.29 |
| TPM2 | 0.9090 | 0.99 | 0.8150 | 1.03 | 0.9717 | 1.01 | 0.8859 | 1.03 | 0.2451 | 0.58 |
| UBE2Z | 0.0001 | 1.74 | 0.0000 | 1.79 | 0.0144 | 1.78 | 0.0976 | 1.41 | 0.2703 | 0.24 |
| UBTF | 0.0538 | 1.39 | 0.0119 | 1.52 | 0.8610 | 1.07 | 0.0214 | 1.53 | 0.0796 | 0.17 |

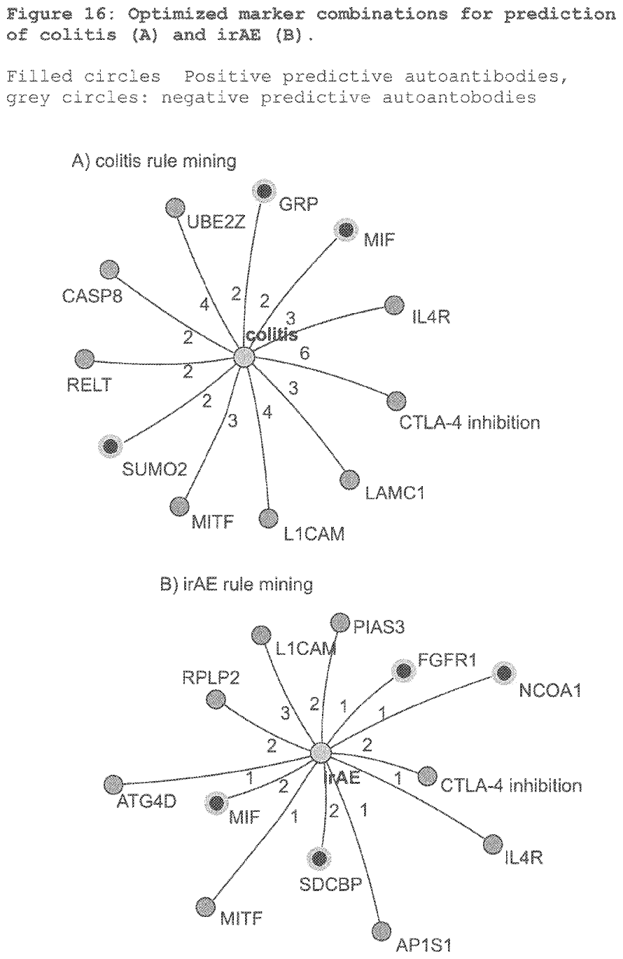

As single markers show very limited sensitivity to predict colitis, we explored association rules of markers exhibiting the highest mutual information for colitis. FIG. 16A shows a set of the best 10 markers for colitis prediction. The sets include markers predicting an increased risk (RELT, CASP8, UBE2Z, IL4R, LAMC1, L1CAM, MITF) but also a reduced risk (SUMO2, GRP, MIF) to develop colitis.

### Example 17: Exploration of an Autoantibody Signature Predicting irAE

A feature ranking approach was applied to select the 15 most important biomarker candidates for irAE shown in FIG. 13. The results of the Cox regression analysis and the associated hazard risk for developing an irAE in patients with high autoantibody levels is shown in Table 11.

The 15 most important autoantibody specificities for predicting an irAE are: PIAS3, RPLP2, NCOA1, ATG4D, KRT7, MIF, TPM2, GABARAPL2, SDCBP, MUM1, MAGEB4, CTSW, SPA17, FGFR1, KRT19.

Seven antigens were associated with an increased risk of irAE (PIAS3, RPLP2, ATG4D, KRT7, TPM2, GABARAPL2, and MAGEB4) and six antigens were associated with a reduced risk of irAE (NCOA1, MIF, SDCB4, MUM1, FGFR1, and KRT19). Therapy-related differences were also observed, for example, KRT7 and FN1 were only predictive in anti-PD-1 treated patients, whereas MAGEB4 and MAGED2 were preferentially predictive in anti-CTLA-4 therapies.

The top biomarker for irAE associated with anti-CTLA-4 therapy was PIAS3 with significant associations found for the all treatment group (HR 1.29, p=0.0001), the ipi-ever group (HR 1.29, p=0.0002; HR 1.35) and the ipi/nivo group (HR 1.32, p=0.0035).

The top candidate for therapies involving anti-PD1 therapy was KRT7 with significant associations found for the ipi/nivo group (HR 1.31, p=0.04) and pembro-never-ipi group (HR 1.55, p=0.0008).

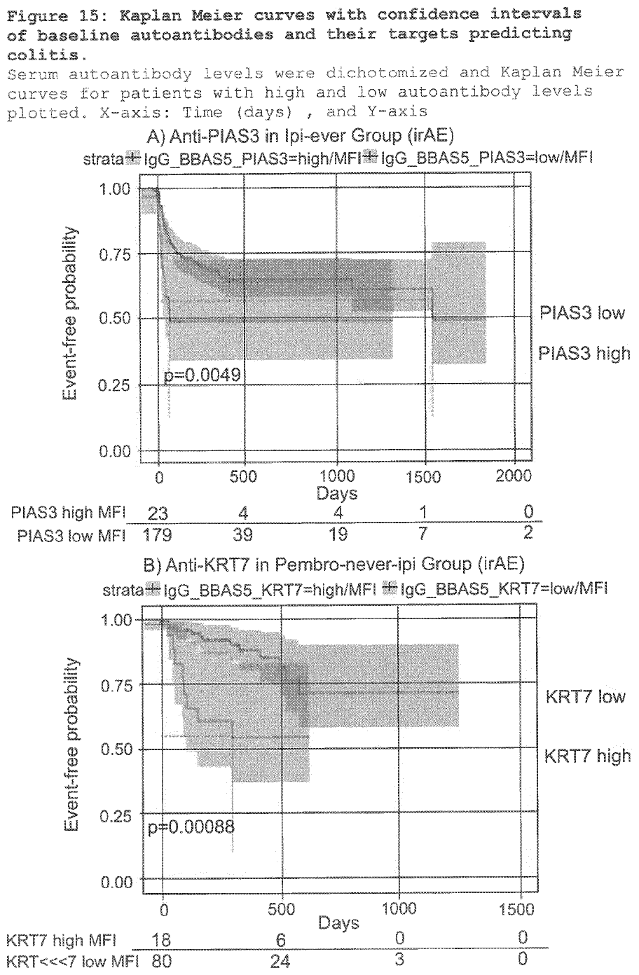

FIG. 15 shows examples of Kaplan-Meier curves for PIAS3 and KRT7.

Therapy-related differences were found for autoantibodies predicting a reduced risk of irAE. Whereas MUM1 (HR 0.69, p=0.0074) and FGFR1 (HR 0.69, p=0.037) were associated with anti-CTLA-4 therapy (Ipi-ever group), MIF1 predicted a reduced risk of irAE for the pembro-never-ipi group (HR 0.49, p=0.032).

TABLE 11
Results of Cox regression analysis of autoantibodies predicting iRAE

| Gene | All Treatments | | Ipi Ever | | Ipi Mono | | Ipi/Nivo | | Pembro Never Ipi | |
|---|---|---|---|---|---|---|---|---|---|---|
| Symbol | P-value | HR | P-value | HR | P-value | HR | P-value | HR | P-value | HR |
| AKT2 | 0.3314 | 1.16 | 0.0328 | 1.44 | 0.0016 | 2.00 | 0.1799 | 1.54 | 0.2841 | 0.64 |
| AMPH | 0.1705 | 0.92 | 0.6275 | 0.97 | 0.0120 | 0.78 | 0.0835 | 1.24 | 0.1184 | 0.82 |
| AP1S1 | 0.1047 | 1.26 | 0.0341 | 1.39 | 0.0674 | 1.50 | 0.5742 | 1.16 | 0.4057 | 0.72 |
| AP2B1 | 0.0991 | 1.17 | 0.0577 | 1.20 | 0.9027 | 1.03 | 0.0578 | 1.22 | 0.8031 | 0.92 |
| ATG4D | 0.0007 | 1.38 | 0.0104 | 1.47 | 0.0002 | 2.85 | 0.7261 | 1.14 | 0.0102 | 1.41 |
| BAG6 | 0.4876 | 0.95 | 0.2034 | 0.88 | 0.1664 | 0.78 | 0.6080 | 0.93 | 0.4482 | 1.12 |
| BICD2 | 0.2381 | 1.12 | 0.3004 | 1.12 | 0.9776 | 0.99 | 0.2951 | 1.17 | 0.4416 | 1.14 |
| BTBD2 | 0.0059 | 1.27 | 0.0112 | 1.27 | 0.1549 | 1.26 | 0.0615 | 1.25 | 0.1832 | 1.37 |
| CASP8 | 0.0216 | 1.21 | 0.0210 | 1.22 | 0.2165 | 1.16 | 0.6609 | 1.09 | 0.7828 | 1.07 |
| CFB | 0.8012 | 1.07 | 0.5560 | 1.18 | 0.0829 | 1.71 | 0.7846 | 0.87 | 0.5894 | 0.68 |
| CTSW | 0.9341 | 0.99 | 0.2161 | 0.84 | 0.7345 | 0.93 | 0.0459 | 0.56 | 0.0286 | 1.40 |
| FGA | 0.8311 | 1.04 | 0.6930 | 1.08 | 0.3998 | 1.28 | 0.6633 | 1.15 | 0.8176 | 0.90 |
| FGFR1 | 0.0092 | 0.64 | 0.0366 | 0.69 | 0.1384 | 0.71 | 0.3325 | 0.71 | 0.1673 | 0.56 |
| FN1 | 0.7124 | 0.96 | 0.0883 | 0.75 | 0.2792 | 0.74 | 0.1251 | 0.58 | 0.0135 | 1.58 |
| GABARAPL2 | 0.0326 | 1.35 | 0.0049 | 1.66 | 0.0112 | 1.90 | 0.6362 | 1.22 | 0.7671 | 1.14 |
| GPHN | 0.5741 | 0.93 | 0.0867 | 0.76 | 0.8016 | 0.95 | 0.0756 | 0.60 | 0.0074 | 1.63 |
| GRP | 0.1150 | 0.91 | 0.3888 | 0.94 | 0.9402 | 1.01 | 0.4915 | 0.92 | 0.2440 | 0.84 |
| IL23A | 0.7794 | 0.96 | 0.1160 | 0.76 | 0.0775 | 0.53 | 0.6231 | 0.88 | 0.0136 | 1.47 |
| IL3 | 0.1571 | 1.15 | 0.3842 | 1.10 | 0.2913 | 1.23 | 0.3890 | 0.83 | 0.3693 | 1.28 |
| IL4R | 0.6152 | 1.11 | 0.3383 | 1.23 | 0.2050 | 1.44 | 0.4637 | 1.37 | 0.4812 | 0.68 |
| KDM4A | 0.0594 | 1.20 | 0.0698 | 1.22 | 0.3602 | 1.27 | 0.8725 | 0.97 | 0.4712 | 1.15 |
| KRT19 | 0.0293 | 0.81 | 0.0275 | 0.77 | 0.1589 | 0.74 | 0.0176 | 0.59 | 0.8006 | 0.96 |
| KRT7 | 0.0491 | 1.15 | 0.5026 | 1.06 | 0.2201 | 0.84 | 0.0396 | 1.31 | 0.0008 | 1.55 |
| L1CAM | 0.0725 | 1.19 | 0.1297 | 1.19 | 0.0689 | 1.41 | 0.9284 | 0.98 | 0.3973 | 1.20 |
| LAMC1 | 0.0242 | 1.13 | 0.0003 | 1.26 | 0.1425 | 1.15 | 0.0819 | 1.22 | 0.0763 | 0.80 |
| LEPR | 0.4214 | 1.05 | 0.3180 | 1.07 | 0.6410 | 1.05 | 0.0425 | 1.24 | 0.6934 | 0.96 |
| MAGEB4 | 0.0347 | 1.17 | 0.0022 | 1.28 | 0.0001 | 1.60 | 0.3936 | 1.11 | 0.5817 | 0.91 |
| MAGED2 | 0.0517 | 1.17 | 0.0069 | 1.24 | 0.0281 | 1.28 | 0.4067 | 1.10 | 0.0867 | 0.49 |
| MIF | 0.0195 | 0.70 | 0.1566 | 0.79 | 0.5506 | 0.89 | 0.3626 | 0.73 | 0.0320 | 0.49 |
| MITF | 0.1663 | 1.08 | 0.4909 | 1.05 | 0.2028 | 0.86 | 0.0060 | 1.28 | 0.1509 | 1.18 |
| MUM1 | 0.0259 | 0.78 | 0.0074 | 0.69 | 0.0787 | 0.69 | 0.2835 | 0.81 | 0.4382 | 1.15 |
| NCOA1 | 0.0092 | 0.85 | 0.0363 | 0.87 | 0.0166 | 0.74 | 0.7861 | 0.97 | 0.0233 | 0.68 |
| PCDH1 | 0.6480 | 0.98 | 0.7551 | 0.98 | 0.3527 | 0.92 | 0.2217 | 1.13 | 0.5825 | 0.94 |
| PIAS3 | 0.0001 | 1.29 | 0.0002 | 1.29 | 0.3222 | 1.14 | 0.0035 | 1.32 | 0.6339 | 1.09 |
| PRKCI | 0.0476 | 1.16 | 0.0454 | 1.18 | 0.3070 | 1.15 | 0.1695 | 1.16 | 0.5245 | 1.14 |
| RELT | 0.3255 | 1.06 | 0.1089 | 1.11 | 0.0555 | 1.19 | 0.4862 | 0.88 | 0.2962 | 0.85 |
| RPLP0 | 0.0200 | 1.28 | 0.2950 | 1.14 | 0.3784 | 1.15 | 0.1426 | 1.40 | 0.0073 | 1.74 |
| RPLP2 | 0.0013 | 1.37 | 0.0073 | 1.35 | 0.3761 | 1.21 | 0.0025 | 1.75 | 0.1249 | 1.40 |
| SDCBP | 0.0106 | 0.66 | 0.0624 | 0.71 | 0.0275 | 0.52 | 0.8785 | 0.97 | 0.1147 | 0.55 |
| SPA17 | 0.9909 | 1.00 | 0.2617 | 1.07 | 0.1289 | 0.85 | 0.0165 | 1.25 | 0.0026 | 0.61 |
| SPTBN1 | 0.2718 | 1.08 | 0.1657 | 1.11 | 0.7897 | 1.03 | 0.4152 | 1.11 | 0.1780 | 0.75 |
| SUMO2 | 0.0215 | 0.80 | 0.0571 | 0.81 | 0.0019 | 0.46 | 0.2611 | 0.85 | 0.1995 | 0.76 |
| TMEM98 | 0.0389 | 0.87 | 0.0147 | 0.82 | 0.0612 | 0.78 | 0.9159 | 1.01 | 0.2175 | 1.17 |
| TP53 | 0.5806 | 1.06 | 0.3057 | 0.86 | 0.9439 | 0.99 | 0.1028 | 0.60 | 0.0863 | 1.21 |
| TPM2 | 0.0648 | 1.14 | 0.0007 | 1.33 | 0.0562 | 1.29 | 0.0085 | 1.38 | 0.0612 | 0.72 |
| UBE2Z | 0.0558 | 1.25 | 0.0136 | 1.35 | 0.1463 | 1.34 | 0.5452 | 1.11 | 0.4863 | 0.74 |
| UBTF | 0.6388 | 1.07 | 0.1961 | 1.21 | 0.8329 | 0.94 | 0.1965 | 1.23 | 0.0961 | 0.47 |

As single markers show very limited sensitivity to predict irAE, we explored association rules of markers exhibiting the highest mutual information for irAE. FIG. 16B shows a set of the best markers for irAE prediction. The sets include markers predicting an increased risk (IL4R, L1CAM, MITF, PIAS3, AP1S1, ATG4D, RPLP2) but also a reduced risk (MIF, NCOA1, FGFR1, SDCBP) to develop an irAE.

### Example 18: Development of Optimized Marker Panels for Colitis

As single markers show very limited sensitivity to predict an adverse event, we explored association rules of markers exhibiting the highest mutual information for colitis. FIG. 16a shows a set of the best 10 markers for colitis prediction. The sets include markers predicting an increased risk (RELT, CASP8, UBE2Z, IL4R, LAMC1, L1CAM, MITF) but also a reduced risk (SUMO2, GRP, MIF) to develop colitis.

### Example 19: Development of Optimized Marker Panels for irAE

Association rules mining was applied to identify optimized marker panels exhibiting the highest mutual information for irAE. FIG. 16b shows a set of the best markers for irAE prediction. The sets include markers predicting an increased risk (IL4R, L1CAM, MITF, PIAS3, AP1S1, ATG4D, RPLP2) but also a reduced risk (MIF, NCOA1, FGFR1, SDCBP) to develop an irAE.

### REFERENCES

Bei, R., Masuelli, L., Palumbo, C., Modesti, M., and Modesti, A. (2009). A common repertoire of autoantibodies is shared by cancer and autoimmune disease patients: Inflammation in their induction and impact on tumor growth. Cancer Lett. 281, 8-23.
Bresnick, A. R., Weber, D. J., and Zimmer, D. B. (2015). 5100 proteins in cancer. Nat. Rev. Cancer 15, 96-109.
Buchbinder, E. I., and Desai, A. (2016). CTLA-4 and PD-1 Pathways: Similarities, Differences, and Implications of Their Inhibition. Am. J. Clin. Oncol. 39, 98-106.
Budde, P., Zucht, H.-D., Vordenbaumen, S., Goehler, H., Fischer-Betz, R., Gamer, M., Marquart, K., Rengers, P., Richter, J., Lueking, A., et al. (2016). Multiparametric detection of autoantibodies in systemic lupus erythematosus. Lupus 25, 812-822.
Callahan, M. K., Postow, M. A., and Wolchok, J. D. (2013). Immunomodulatory therapy for melanoma: Ipilimumab and beyond. Clin. Dermatol. 31, 191-199.
Chiaruttini, G., Mele, S., Opzoomer, J., Crescioli, S., Ilieva, K. M., Lacy, K. E., and Karagiannis, S. N. (2017). B cells and the humoral response in melanoma: The overlooked players of the tumor microenvironment. OncoImmunology 6, e1294296.
Cuda, C. M., Pope, R. M., and Perlman, H. (2016). The inflammatory role of phagocyte apoptotic pathways in rheumatic diseases. Nat. Rev. Rheumatol. 12, 543-558.
Dai, N., Ji, F., Wright, J., Minichiello, L., Sadreyev, R., and Avruch, J. (2017). IGF2 mRNA binding protein-2 is a tumor promoter that drives cancer proliferation through its client mRNAs IGF2 and HMGA1. ELife 6.
Das, S. K., Bhutia, S. K., Kegelman, T. P., Peachy, L., Oyesanya, R. A., Dasgupta, S., Sokhi, U.K., Azab, B., Dash, R., Quinn, B. A., et al. (2012). MDA-9/syntenin: a positive gatekeeper of melanoma metastasis. Front. Biosci. Landmark Ed. 17, 1-15.
Eisenhauer, E. A., Therasse, P., Bogaerts, J., Schwartz, L. H., Sargent, D., Ford, R., Dancey, J., Arbuck, S., Gwyther, S., Mooney, M., et al. (2009). New response evaluation criteria in solid tumours: Revised RECIST guideline (version 1.1). Eur. J. Cancer 45, 228-247.
Ferrer, C. M., Sodi, V. L., and Reginato, M. J. (2016). 0-GlcNAcylation in Cancer Biology: Linking Metabolism and Signaling. J. Mol. Biol. 428, 3282-3294.
Garg, H., Suri, P., Gupta, J. C., Talwar, G. P., and Dubey, S. (2016). Survivin: a unique target for tumor therapy. Cancer Cell Int. 16.
Gulley, J. L., Madan, R. A., Tsang, K. Y., Jochems, C., Marté, J. L., Farsaci, B., Tucker, J. A., Hodge, J. W., Liewehr, D. J., Steinberg, S. M., et al. (2014). Immune Impact Induced by PROSTVAC (PSA-TRICOM), a Therapeutic Vaccine for Melanoma. Cancer Immunol. Res. 2, 133-141.
Hodi, F. S., Chesney, J., Pavlick, A. C., Robert, C., Grossmann, K. F., McDermott, D. F., Linette, G. P., Meyer, N., Giguere, J. K., Agarwala, S. S., et al. (2016). Combined nivolumab and ipilimumab versus ipilimumab alone in patients with advanced melanoma: 2-year overall survival outcomes in a multicentre, randomised, controlled, phase 2 trial. Lancet Oncol. 17, 1558-1568.
Houles, T., and Roux, P. P. (2017). Defining the role of the RSK isoforms in cancer. Semin. Cancer Biol.
Kindt, N., Journe, F., Laurent, G., and Saussez, S. (2016). Involvement of macrophage migration inhibitory factor in cancer and novel therapeutic targets. Oncol. Lett. 12, 2247-2253.
Larkin, J., Chiarion-Sileni, V., Gonzalez, R., Grob, J. J., Cowey, C. L., Lao, C. D., Schadendorf, D., Dummer, R., Smylie, M., Rutkowski, P., et al. (2015). Combined Nivolumab and Ipilimumab or Monotherapy in Untreated Melanoma. N. Engl. J. Med. 373, 23-34.
Luo, L. Y., and Hahn, W. C. (2015). Oncogenic Signaling Adaptor Proteins. J. Genet. Genomics 42, 521-529.
Manson, G., Norwood, J., Marabelle, A., Kohrt, H., and Houot, R. (2016). Biomarkers associated with checkpoint inhibitors. Ann. Oncol. 27, 1199-1206.
Moscat, J., Karin, M., and Diaz-Meco, M. T. (2016). p62 in Cancer: Signaling Adaptor Beyond Autophagy. Cell 167, 606-609.
Peng, S., Wang, K., Gu, Y., Chen, Y., Nan, X., Xing, J., Cui, Q., Chen, Y., Ge, Q., and Zhao, H. (2015). TRAF3IP3, a novel autophagy up-regulated gene, is involved in marginal zone B lymphocyte development and survival. Clin. Exp. Immunol. 182, 57-68.
Robin, X., Turck, N., Hainard, A., Tiberti, N., Lisacek, F., Sanchez, J.-C., and Müller, M. (2011). pROC: an open-source package for R and S+ to analyze and compare ROC curves. BMC Bioinformatics 12, 77.
Shi, Y., Feng, Y., Kang, J., Liu, C., Li, Z., Li, D., Cao, W., Qiu, J., Guo, Z., Bi, E., et al. (2007). Critical regulation of CD4+ T cell survival and autoimmunity by beta-arrestin 1. Nat. Immunol. 8, 817-824.
Smolinsky, B., Eichler, S. A., Buchmeier, S., Meier, J. C., and Schwarz, G. (2008). Splice-specific Functions of Gephyrin in Molybdenum Cofactor Biosynthesis. J. Biol. Chem. 283, 17370-17379.
Spain, L., Diem, S., and Larkin, J. (2016). Management of toxicities of immune checkpoint inhibitors. Cancer Treat. Rev. 44, 51-60.
Topalian, S. L., Taube, J. M., Anders, R. A., and Pardoll, D. M. (2016). Mechanism-driven biomarkers to guide immune checkpoint blockade in cancer therapy. Nat. Rev. Cancer 16, 275-287.
Tusher, V. G., Tibshirani, R., and Chu, G. (2001). Significance analysis of microarrays applied to the ionizing radiation response. Proc. Natl. Acad. Sci. U.S.A 98, 5116-5121.
Uehara, T., Kage-Nakadai, E., Yoshina, S., Imae, R., and Mitani, S. (2015). The Tumor Suppressor BCL7B Functions in the Wnt Signaling Pathway. PLoS Genet. 11, e1004921.

1. A method of identifying a tumor-associated antigen (TAA) for melanoma comprising:
a) selecting a group of patients with melanoma and a group of patients who are healthy,
b) assaying the level of an autoantibody to an antigen in a sample from a patient in the group,
c) comparing the level of the autoantibody from the patient in the group or the group of patients with melanoma to the level of the autoantibody in the group of healthy patients, and
d) determining that the antigen is a TAA for melanoma if the level of the autoantibody to the antigen is statistically different between the group of patients with melanoma versus the group of healthy patients.

2. The method of claim 1, wherein the antigen is an antigen encoded by a gene listed in Table 1 or Table 9, or wherein the antigen comprises an amino acid sequence of any one of SEQ ID NOS: 1-169.

3. The method of claim 1, wherein the TAA is encoded by a gene listed in Table 2.

4. The method of claim 1, wherein the assaying comprises b1) contacting a portion of serum from the patient with a sample of an antigen immobilized onto a solid support.

5. The method of claim 4, wherein the solid support is a bead.

6. The method of claim 5, wherein the bead is a microsphere.

7. A method of identifying a tumor-associated antigen (TAA) as a marker for melanoma overall survival (MOS) or melanoma disease control rate (MDCR) comprising:
a) selecting a first group of patients with melanoma who have statistically greater MOS or MDCR than a second group of patients with melanoma,
b) assaying the level of an autoantibody to the antigen in a sample from each of the patients in the first group,
c) comparing the level of the autoantibody to the antigen in each of the patients in the first group to the level of the autoantibody in each of the patients in the second group, and
d) determining that the antigen is a TAA marker for MOS or MDCR if the level of the autoantibody to the antigen is statistically different between the first group of patients and the second group of patients.

8. The method of claim 7, wherein the antigen is encoded by a gene listed in Table 3.

9. The method of claim 7, wherein the TAA marker for MOS or MDCR is encoded by a gene listed in Table 3.

10. The method of claim 7, wherein the assaying comprises b1) contacting a portion of serum from the patient with a sample of an antigen immobilized onto a solid support.

11. The method of claim 10, wherein the solid support is a bead.

12. The method of claim 11, wherein the bead is a microsphere.

13. A method of identifying and treating a melanoma patient susceptible to an immune-related adverse event (irAE) after treatment with a checkpoint inhibitor comprising:
a) determining the level of one or more antigens encoded by a gene listed in Table 4 having a positive value for SAM Fold.Change,
b) assaying the level of one or more antigens in a sample from a melanoma patient,
c) comparing the level of the one or more antigens with an average level of the one or more antigens for a group of patients with melanoma, and
d) administering the checkpoint inhibitor to the melanoma patient if (a) the level of the one or more antigens encoded by a gene listed in Table 4 having a value for SAM Fold.Change>1 in the patient is less than the average level of the one or more antigens encoded by a gene listed in Table 4 having a value for SAM Fold.Change>1 in the group of patients with melanoma or (b) the level of the one or more antigens encoded by a gene listed in Table 4 having a value for SAM Fold.Change<=1 in the patient is greater than the average level of the one or more antigens encoded by a gene listed in Table 4 having a value for SAM Fold.Change<=1 in the group of patients with melanoma.

14-22. (canceled)

23. A method of identifying and treating a melanoma patient with a checkpoint inhibitor comprising:
a) determining the level of one or more antigens encoded by a gene listed in Table 5 having a value for SAMR Fold.Change>1,
b) comparing the level of the one or more antigens with an average level of the one or more antigens for a group of patients with melanoma, and
c) administering the checkpoint inhibitor if the level of the one or more antigens in the patient is greater than the average level of the one or more antigens in the group of patients with melanoma.

24-32. (canceled)

33. A method of identifying and treating a melanoma patient with Ipilimumab comprising:
a) determining the level of one or more antigens encoded by a gene listed in Table 6 having a value for SAMR Fold.Change>1,
b) comparing the level of the one or more antigens with an average level of the one or more antigens for a group of patients with melanoma, and
c) administering the Ipilimumab if the level of the one or more antigens in the patient is less than the average level of the one or more antigens in the group of patients with melanoma.

34. A method of identifying and treating a melanoma patient with Ipilimumab comprising:
a) determining the level of one or more antigens encoded by a gene listed in Table 6 having a value for SAMR Fold.Change<=1,
b) comparing the level of the one or more antigens with an average level of the one or more antigens for a group of patients with melanoma, and
c) administering the Ipilimumab if the level of the one or more antigens in the patient is greater than the average level of the one or more antigens in the group of patients with melanoma.

35-39. (canceled)

40. A method of identifying and treating a melanoma patient with a CTLA-4 inhibitor comprising:
a) determining the level of one or more antigens encoded by a gene listed in Table 6 having a value for SAMR Fold.Change>1,
b) comparing the level of the one or more antigens with an average level of the one or more antigens for a group of patients with melanoma, and
c) administering the CTLA-4 inhibitor if the level of the one or more antigens in the patient is less than the average level of the one or more antigens in the group of patients with melanoma.

41. A method of identifying and treating a melanoma patient with a CTLA-4 inhibitor comprising:
a) determining the level of one or more antigens encoded by a gene listed in Table 6 having a value for SAMR Fold.Change<=1,
b) comparing the level of the one or more antigens with an average level of the one or more antigens for a group of patients with melanoma, and
c) administering the CTLA-4 inhibitor if the level of the one or more antigens in the patient is greater than the average level of the one or more antigens in the group of patients with melanoma.

42-46. (canceled)

47. A method of identifying and treating a melanoma patient with a PD-1/PD-L1 pathway inhibitor comprising:
a) determining the level of one or more antigens encoded by a gene listed in Table 7 having a value for SAMR Fold.Change>1,
b) comparing the level of the one or more antigens with an average level of the one or more antigens for a group of patients with melanoma, and
c) administering the PD-1/PD-L1 pathway inhibitor if the level of the one or more antigens in the patient is greater than the average level of the one or more antigens in the group of patients with melanoma.

48-54. (canceled)

55. A method of identifying and treating a melanoma patient with pembrolizumab comprising:
a) determining the level of one or more antigens encoded by a gene listed in Table 8 having a value for SAMR Fold.Change<=1,
b) comparing the level of the one or more antigens with an average level of the one or more antigens for a group of patients with melanoma, and
c) administering the pembrolizumab if the level of the one or more antigens in the patient is greater than the average level of the one or more antigens in the group of patients with melanoma.

56. A method of identifying and treating a melanoma patient with pembrolizumab comprising:
a) determining the level of one or more antigens encoded by a gene listed in Table 8 having a value for SAMR Fold.Change>1,
b) comparing the level of the one or more antigens with an average level of the one or more antigens for a group of patients with melanoma, and
c) administering the pembrolizumab if the level of the one or more antigens in the patient is less than the average level of the one or more antigens in the group of patients with melanoma.

57-61. (canceled)

62. A method of identifying an antigen predictive of development of an irAE or colitis in a patient with melanoma who is treated with a checkpoint inhibitor comprising:
a) selecting a first group of patients with melanoma who developed irAE or colitis after treatment with the checkpoint inhibitor,
b) assaying the level of an autoantibody to an antigen in a sample from a patient in the first group who developed irAE or colitis,
c) comparing the level of the autoantibody from the patient in the first group to the level of the autoantibody in a second group of patients with melanoma after treatment with the checkpoint inhibitor but did not develop irAE or colitis, and
d) determining that the antigen is predictive of development of an irAE or colitis if the level of the autoantibody to the antigen is statistically different between the first group of patients and the second group of patients.

63-66. (canceled)

67. A method of identifying an antigen predictive of development of colitis in a patient with melanoma who is treated with a checkpoint inhibitor comprising:
a) selecting a first group of patients with melanoma who developed colitis after treatment with the checkpoint inhibitor,
b) assaying the level of an autoantibody to an antigen in a sample from a patient in the first group who developed colitis,
c) comparing the level of the autoantibody from the patient in the first group to the level of the autoantibody in a second group of patients with melanoma after treatment with the checkpoint inhibitor but did not develop colitis, and
d) determining that the antigen is predictive of development of colitis if the level of the autoantibody to the antigen is statistically different between the first group of patients and the second group of patients.

68-78. (canceled)

79. A method of identifying an antigen predictive of development of an irAE in a patient with melanoma who is treated with a checkpoint inhibitor comprising:
a) selecting a first group of patients with melanoma who developed irAE after treatment with the checkpoint inhibitor,
b) assaying the level of an autoantibody to an antigen in a sample from a patient in the first group who developed the irAE,
c) comparing the level of the autoantibody from the patient in the first group to the level of the autoantibody in a second group of patients with melanoma after treatment with the checkpoint inhibitor but did not develop the irAE, and
d) determining that the antigen is predictive of development of the irAE if the level of the autoantibody to the antigen is statistically different between the first group of patients and the second group of patients.

80-85. (canceled)
